(19) United States
(12) Patent Application Publication
(10) Pub. No.: US 2025/0212879 A1
(43) Pub. Date: Jul. 3, 2025

(54) HERBICIDAL COMPOUNDS

(71) Applicant: SYNGENTA CROP PROTECTION AG, Basel (CH)

(72) Inventor: William Guy WHITTINGHAM, Bracknell, Berkshire (GB)

(73) Assignee: SYNGENTA CROP PROTECTION AG, Basel (CH)

(21) Appl. No.: 18/845,744

(22) PCT Filed: Mar. 6, 2023
(86) PCT No.: PCT/EP2023/055564

§ 371 Date: Sep. 10, 2024

(30) Foreign Application Priority Data
Mar. 11, 2022 (EP) 22161532.1

(51) Int. Cl. A01N 43/40; A01N 37/16; A01N 43/60; A01P 13/02; C07C 69/78; C07D 213/61; C07D 213/89; C07D 241/12

(52) U.S. Cl. CPC A01N 43/40; A01N 37/16; A01N 43/60; A01P 13/02; C07C 69/78; C07D 213/61; C07D 213/89; C07D 241/12

(57) ABSTRACT

Compounds of the formula (I) wherein the substituents are as defined in claim 1, useful as a pesticides, especially as herbicides.

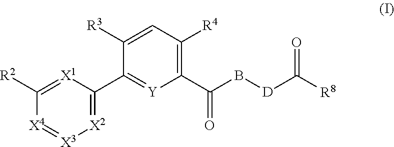

The present invention relates to herbicidally active compounds, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

WO 2002/42275 and U.S. Pat. No. 6,010,980 both disclose herbicidally active 2-substituted pyridine compounds.

The present invention is based on the finding that compounds of formula (I) as defined herein, exhibit surprisingly good herbicidal activity. Thus, according to the present invention there is provided a compound of formula (I) or an agronomically acceptable salt thereof:

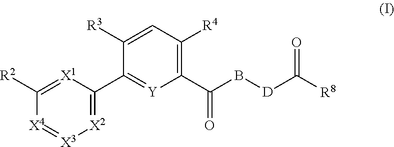

wherein
X¹ is selected from C—R¹, nitrogen and N⁺—O⁻;
X² is selected from C—R¹⁷ and nitrogen;
X³ is selected from C—R¹⁸ and nitrogen;
X⁴ is selected from C—R¹⁹ and nitrogen;
with the proviso that a maximum of two of X¹, X², X³ and X⁴ are nitrogen, and X³ and X⁴ are not both nitrogen;
Y is selected from C—H and nitrogen;
B is selected from O, S and NR⁵;
D is (CR⁶R⁷)ₙ;
n is an integer from 1 to 4;
R¹ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹;
R² is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹; or
R¹ and R² together with the carbon atoms to which they are attached form a 5- or 6-membered ring, which may be saturated or partially or fully unsaturated, and which may optionally contain one or two heteroatoms selected from nitrogen, oxygen and sulfur, and which may be substituted with 1 to 4 groups represented by R¹⁶; or
R² and R¹⁹ together with the carbon atoms to which they are attached form a 5- or 6-membered ring, which may be saturated or partially or fully unsaturated, and which may optionally contain one or two heteroatoms selected from nitrogen, oxygen and sulfur, and which may be substituted with 1 to 4 groups represented by R¹⁶;
R³ is selected from hydrogen, halogen, C₁-C₄alkyl, C₁-C₄haloalkyl, C₁-C₄alkoxy, C₁-C₄haloalkoxy, C₁-C₄alkylthio, and C₁-C₄alkylsulfonyl;
R⁴ is selected from hydrogen, halogen, cyano, nitro, aminocarbonyl, aminothiocarbonyl, C₁-C₄alkyl, C₁-C₄haloalkyl, C₁-C₄alkoxy, C₁-C₄haloalkoxy, and C₁-C₄alkylsulfonyl;
R⁵ is selected from the group consisting of hydrogen, hydroxy, C₁-C₆alkyl, and C₁-C₄alkoxy;
each R⁶ and R⁷ is independently selected from hydrogen, halogen, C₁-C₆alkyl, C₁-C₆haloalkyl, hydroxy, C₁-C₄alkoxy, C₁-C₄alkoxycarbonyl, and CH₂OR¹²; provided that R⁶ and R⁷ are not both hydroxy on the same carbon atom;
or two groups R⁶ and R⁷, on the same or different carbon atoms, together form a C₁-C₅alkylene chain, which contain 0, 1 or 2 oxygen atoms, substituted by 1 to 3 groups represented by R¹⁵;
or two groups R⁶ and R⁷, on the same carbon atom, together with the carbon atom to which they are attached may form a C₂alkene;
R⁸ is selected from OR⁹, SR⁹, and NR¹⁰R¹¹; R⁹ is selected from hydrogen, C₁-C₁₀alkyl, C₁-C₁₀haloalkyl, C₃-C₆alkenyl, C₃-C₆haloalkenyl, C₃-C₆alkynyl, C₁-C₄alkoxyC₁-C₆alkyl, C₁-C₄haloalkoxyC₁-C₆alkyl, C₆-C₁₀arylC₁-C₃alkyl, C₆-C₁₀arylC₁-C₃alkyl substituted by 1 to 4 groups represented by R¹³, heteroarylC₁-C₃alkyl, and heteroarylC₁-C₃alkyl substituted by 1 to 3 groups represented by R¹³;
R¹⁰ is selected from hydrogen, C₁-C₆alkyl, and SO₂R¹⁴;
R¹¹ is selected from hydrogen and C₁-C₆alkyl; or
R¹⁰ and R¹¹ together with the nitrogen atom to which they are attached form a 3- to 6-membered heterocyclyl ring, which optionally contains an oxygen atom;
R¹² is selected from hydrogen, C₁-C₄alkyl, and C₁-C₄alkylcarbonyl;
each R¹³ is independently selected from halogen, C₁-C₄alkyl, C₁-C₄haloalkyl, C₁-C₄alkoxy, C₁-C₄haloalkoxy, cyano, and C₁-C₄alkylsulfonyl;
R¹⁴ is selected from C₁-C₄alkyl, C₁-C₄haloalkyl, and C₁-C₄alkyl(C₁-C₄alkyl)amino;
each R¹⁵ is independently selected from hydrogen, halogen, C₁-C₄alkyl, and C₁-C₄haloalkyl;
R¹⁶ is selected from halogen, C₁-C₄alkyl, C₁-C₄haloalkyl, C₁-C₄alkoxy, C₁-C₄haloalkoxy, cyano, and C₁-C₄alkylsulfonyl;
R¹⁷ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹;
R¹⁸ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹;
R¹⁹ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹; and
with the proviso that R¹, R², R¹⁷, R¹⁸ and R¹⁹ are not all hydrogen.

According to a second aspect of the invention, there is provided an agrochemical composition comprising a herbicidally effective amount of a compound of formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

According to a fourth aspect of the invention, there is provided the use of a compound of formula (I) as a herbicide.

According to a fifth aspect of the invention, there is provided a process for the preparation of compounds of formula (I).

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, amino means an —NH₂ group.

As used herein, the term “C₁-C₁₀alkyl” refers to a straight or branched hydrocarbon chain radical consisting solely of carbon and hydrogen atoms, containing no unsaturation, having from one to ten carbon atoms, and which is attached to the rest of the molecule by a single bond. C₁-C₆alkyl, C₁-C₄alkyl and C₁-C₂alkyl are to be construed accordingly. Examples of C₁-C₁₀alkyl include, but are not limited to, methyl (Me), ethyl (Et), n-propyl, 1-methylethyl (iso-propyl), n-butyl, and 1-dimethylethyl (t-butyl).

A “C₁-C₆alkylene” group refers to the corresponding definition of C₁-C₆alkyl, except that such radical is attached to the rest of the molecule by two single bonds. The term “C₁-C₂alkylene” is to be construed accordingly. Examples of C₁-C₆alkylene, include, but are not limited to, —CH₂—, —CH₂CH₂— and —(CH₂)₃—.

As used herein, the term “C₁-C₄alkoxy” refers to a radical of the formula —ORₐ where Rₐ is a C₁-C₄alkyl radical as generally defined above. C₁-C₃alkoxy is to be construed accordingly. Examples of C₁-C₄alkoxy include, but are not limited to, methoxy, ethoxy, propoxy, iso-propoxy and t-butoxy.

As used herein, the term “C₁-C₁₀haloalkyl” refers to a C₁-C₁₀alkyl radical as generally defined above substituted by one or more of the same or different halogen atoms. C₁-C₆haloalkyl and C₁-C₄haloalkyl are to be construed accordingly. Examples of C₁-C₁₀haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. C₃-C₆alkenyl and C₂-C₄alkenyl are to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. C₃-C₆haloalkenyl is to be construed accordingly. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₃-C₆alkynyl and C₂-C₄alkynyl are to be construed accordingly. Examples of C₂-C₆alkynyl include, but are not limited to, prop-1-ynyl, propargyl (prop-2-ynyl) and but-1-ynyl.

As used herein, the term “C₁-C₄haloalkoxy” refers to a C₁-C₄alkoxy group as defined above substituted by one or more of the same or different halogen atoms. C₁-C₃haloalkoxy is to be construed accordingly. Examples of C₁-C₄haloalkoxy include, but are not limited to, fluoromethoxy, difluoromethoxy, fluoroethoxy, trifluoromethoxy and trifluoroethoxy.

As used herein, the term “C₁-C₄haloalkoxyC₁-C₆alkyl” refers to a radical of the formula R_{b}—O—Rₐ— where R_{b} is a C₁-C₄haloalkyl radical as generally defined above, and Rₐ is a C₁-C₆alkylene radical as generally defined above.

As used herein, the term “C₁-C₄alkoxyC₁-C₆alkyl” refers to a radical of the formula R_{b}—O—Rₐ— where R_{b} is a C₁-C₄alkyl radical as generally defined above, and Rₐ is a C₁-C₆alkylene radical as generally defined above.

As used herein, the term “C₁-C₄alkoxyC₁-C₄alkoxy” refers to a radical of the formula R_{b}—O—Rₐ—O— where R_{b} is a C₁-C₄alkyl radical as generally defined above, and Rₐ is a C₁-C₄alkylene radical as generally defined above.

As used herein, the term “C₁-C₄alkylthio” refers to a radical of the formula —SRₐ, where Rₐ is a C₁-C₄alkyl radical as generally defined above.

As used herein, the term “C₁-C₄alkylsulfonyl” refers to a radical of the formula —S(O)₂Rₐ, where Rₐ is a C₁-C₄alkyl radical as generally defined above. The terms “C₁-C₃alkylsulfonyl” and “C₁-C₂alkylsulfonyl”, are to be construed accordingly. Examples of C₁-C₄alkylsulfonnyl include, but are not limited to methylsulfonyl.

As used herein, the term “C₁-C₆alkylcarbonyl” refers to a radical of the formula —C(O)Rₐ where Rₐ is a C₁-C₆alkyl radical as generally defined above. C₁-C₄alkylcarbonyl is to be construed accordingly.

As used herein, the term “C₁-C₆alkoxycarbonyl” refers to a radical of the formula —C(O)ORₐ where Rₐ is a C₁-C₆alkyl radical as generally defined above. C₁-C₄alkoxycarbonyl is to be construed accordingly.

As used herein, the term “C₁-C₄alkylsulfonyloxy” refers to a radical of the formula —OS(O)₂Rₐ, where Rₐ is a C₁-C₄alkyl radical as generally defined above. The terms “C₁-C₃alkylsulfonyloxy” and “C₁-C₂alkylsulfonyloxy”, are to be construed accordingly.

As used herein, the term “C₁-C₄haloalkylsulfonyl” refers to a radical of the formula —S(O)₂Rₐ, where Rₐ is a C₁-C₄haloalkyl radical as generally defined above.

As used herein, the term “C₁-C₄haloalkylsulfonyloxy” refers to a radical of the formula —OS(O)₂Rₐ, where Rₐ is a C₁-C₄haloalkyl radical as generally defined above.

As used herein, the term “C₁-C₆alkylsulfinyl” refers to a radical of the formula —S(O)Rₐ, where Rₐ is a C₁-C₆alkyl radical as generally defined above. The terms “C₁-C₄alkylsulfinyl” and “C₁-C₃alkylsulfinyl”, are to be construed accordingly. Examples of C₁-C₆alkylsulfinyl include, but are not limited to methylsulfinyl.

As used herein, the term “C₁-C₄haloalkylsulfinyl” refers to a radical of the formula —S(O)Rₐ, where Rₐ is a C₁-C₄haloalkyl radical as generally defined above.

As used herein, the term “C₁-C₄haloalkylthio” refers to a radical of the formula —SRₐ, where Rₐ is a C₁-C₄haloalkyl radical as generally defined above.

As used herein, the term “C₁-C₄alkylamino” refers to a radical of the formula —NHRₐ, where Rₐ is a C₁-C₄alkyl radical as generally defined above.

As used herein, the term “N,N-di(C₁-C₄alkyl)amino” refers to a radical of the formula —N(Rₐ)(R_{b}), wherein Rₐ and R_{b} are each individually a C₁-C₄alkyl radical as generally defined above. The term “N,N-di(C₁-C₃alkyl)amino” is to be construed accordingly.

As used herein, the term “C₁-C₄alkylcarbonylamino” refers to a radical of the formula —NH(O)CRₐ, where Rₐ is a C₁-C₄alkyl radical as generally defined above.

As used herein, the term “C₁-C₄alkylcarbonyl(C₁-C₄alkyl)amino” refers to a radical of the formula —NH(Rₐ)(O)CR_{b}, wherein Rₐ and R_{b} are each individually a C₁-C₄alkyl radical as generally defined above.

As used herein, the term “C₁-C₄alkyl(C₁-C₄alkyl)amino” refers to a radical of the formula —NH(Rₐ)R_{b}, wherein Rₐ and R_{b} are each individually a C₁-C₄alkyl radical as generally defined above.

As used herein, the term “C₁-C₄alkyloxycarbonylamino” refers to a radical of the formula —NH(O)CORₐ, where Rₐ is a C₁-C₄alkyl radical as generally defined above.

As used herein, the term “C₁-C₄alkylaminocarbonylamino” refers to a radical of the formula —NH(O)CNHRₐ, where Rₐ is a C₁-C₄alkyl radical as generally defined above.

As used herein, the term “C₁-C₄alkylsulfonylamino” refers to a radical of the formula RₐS(O)₂NH— where Rₐ is a C₁-C₄alkyl radical as generally defined above.

As used herein, the term “C₁-C₄haloalkylsulfonylamino” refers to a radical of the formula refers to a radical of the formula RₐS(O)₂NH—, where Rₐ is a C₁-C₄haloalkyl radical as generally defined above.

As used herein, the term “aminocarbonyl” refers to a radical of the formula —C(O)NH₂.

As used herein, the term “aminocarbonylamino” refers to a radical of the formula —NH(O)CNH₂.

As used herein, the term “aminothiocarbonyl” refers to a radical of the formula —C(S)NH₂.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₆-C₁₀aryl” refers to a 6- to 10-membered aromatic ring system consisting solely of carbon and hydrogen atoms which may be mono-, bi- or tricyclic. Examples of such ring systems include phenyl, naphthalenyl, or indenyl.

As used herein, the term “C₆-C₁₀arylC₁-C₃alkyl” refers to an aryl moiety as generally defined above, which is attached to the rest of the molecule by a C₁-C₃alkylene linker as defined above.

As used herein, except where explicitly stated otherwise, the term “heteroaryl” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heteroarylC₁-C₃alkyl” refers to a heteroaryl ring as generally defined above attached to the rest of the molecule through a C₁-C₃alkylene linker as defined above.

As used herein, except where explicitly stated otherwise, the term “heterocyclyl” or “heterocyclic” refers to a stable 4- to 6-membered non-aromatic monocyclic ring radical which comprises 1, 2, or 3 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or δ-lactamyl.

As used herein, except where explicitly stated otherwise, the term “heterocyclylC₁-C₃alkyl” refers to a heterocyclyl ring as generally defined above attached to the rest of the molecule through a C₁-C₃alkylene linker as defined above.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

Suitable agronomically acceptable salts of the present invention can be with cations that include but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

The following list provides definitions, including preferred definitions, for substituents X¹, X², X³, X⁴, Y, B, D, n, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ with reference to the compounds of formula (I) according to the invention. For any one of these substituents, any of the definitions given below may be combined with any definition of any other substituent given below or elsewhere in this document.

Preferably X¹ is selected from C—R¹ and N⁺—O⁻, more preferably C—R¹.

Preferably X² is selected from C—R¹⁷ and nitrogen, more preferably nitrogen.

Preferably X³ is selected from C—R¹⁸ and nitrogen, more preferably C—R¹⁸.

Preferably X⁴ is selected from C—R¹⁹ and nitrogen, more preferably C—R¹⁹.

Preferably with the proviso that a maximum of one of X², X³, and X⁴ is nitrogen.

Preferably Y is C—H.

Preferably B is selected from O, NH and NMe, more preferably B is O and NMe, more preferably still B is O.

Preferably n is an integer from 1 to 2, more preferably n is 2.

Preferably R¹ is selected from hydrogen, halogen, C₁-C₄alkyl, C₁-C₄haloalkyl, C₁-C₄alkoxy, C₁-C₄haloalkoxy, more preferably hydrogen, fluorine, chlorine, C₁-C₂alkyl, C₁-C₂haloalkyl, most preferably hydrogen, fluorine, chlorine, methyl and trifluoromethyl.

Preferably R² is selected from hydrogen, halogen, C₁-C₄alkyl, C₁-C₄haloalkyl, C₁-C₄alkoxy, C₁-C₄haloalkoxy, more preferably hydrogen, fluorine, chlorine, C₁-C₂alkyl, C₁-C₂haloalkyl, most preferably hydrogen, fluorine, chlorine, methyl and trifluoromethyl; or Preferably R² and R¹⁹ together with the carbon atoms to which they are attached form a 5-membered saturated ring, optionally containing one or two oxygen atoms, and which may be substituted with 1 to 2 groups represented by R¹⁶.

In one set of embodiments, R² is selected from hydrogen, halogen, cyano, nitro, C₂-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹.

Preferably, R² is selected from hydrogen, halogen, C₂-C₄alkyl, C₁-C₄haloalkyl, C₁-C₄alkoxy, C₁-C₄haloalkoxy. More preferably R² is selected from hydrogen, fluorine, chlorine, C₂-C₄alkyl, and C₁-C₂haloalkyl. More preferably still, R² is selected from hydrogen, fluorine, chlorine, and trifluoromethyl.

Preferably R³ is selected from hydrogen, chlorine and fluorine, more preferably hydrogen and fluorine.

Preferably R⁴ is selected from hydrogen, chlorine, bromine, cyano and aminothiocarbonyl, more preferably chlorine, bromine and cyano, most preferably chlorine.

Preferably each R⁶ and R⁷ is independently selected from hydrogen, halogen, C₁-C₄alkyl and C₁-C₄alkoxycarbonyl, more preferably hydrogen, halogen and C₁-C₂alkyl, most preferably hydrogen, chlorine and methyl.

Preferably R⁸ is OR⁹.

Preferably R⁹ is selected from hydrogen, C₁-C₄alkyl, C₁-C₄haloalkyl, C₁-C₂alkoxyC₁-C₂alkyl, phenylC₁-C₂alkyl and phenylC₁-C₂alkyl substituted by 1 to 2 groups represented by R¹³, more preferably hydrogen, C₁-C₄alkyl, C₁-C₂alkoxyC₁-C₂alkyl and phenylC₁-C₂alkyl, most preferably hydrogen, C₁-C₄alkyl and phenylC₁-C₂alkyl.

In one set of embodiments, R⁹ is selected from C₁-C₄alkyl, C₁-C₄haloalkyl, C₁-C₂alkoxyC₁-C₂alkyl, phenylC₁-C₂alkyl and phenylC₁-C₂alkyl substituted by 1 to 2 groups represented by R¹³, more preferably C₁-C₄alkyl, C₁-C₂alkoxyC₁-C₂alkyl and phenylC₁-C₂alkyl, most preferably C₁-C₄alkyl and phenylC₁-C₂alkyl.

Preferably R¹⁰ is selected from hydrogen and SO₂R¹⁴, more preferably SO₂R¹⁴.

Preferably R¹¹ is hydrogen.

Preferably R¹² is selected from hydrogen, C₁-C₂alkyl and C₁-C₂alkylcarbonyl, more preferably hydrogen and methyl.

Preferably R¹³ is selected from halogen, C₁-C₄alkyl, C₁-C₄haloalkyl, C₁-C₄alkoxy, C₁-C₄haloalkoxy, cyano and C₁-C₄alkylsulfonyl.

Preferably R¹⁴ is selected from C₁-C₄alkyl and C₁-C₄alkyl(C₁-C₄alkyl)amino, more preferably methyl and isopropyl(methyl)amino.

Preferably each R¹⁵ is independently selected from hydrogen, halogen and C₁-C₂alkyl, more preferably hydrogen and methyl, most preferably hydrogen.

Preferably R¹⁶ is halogen, more preferably fluorine.

Preferably R¹⁷ is selected from hydrogen, halogen, C₁-C₄alkyl, C₁-C₄haloalkyl, C₁-C₄alkoxy, C₁-C₄haloalkoxy, more preferably hydrogen, fluorine, chlorine, C₁-C₂alkyl, C₁-C₂haloalkyl, most preferably hydrogen, fluorine, chlorine, methyl and trifluoromethyl.

Preferably R¹⁸ is selected from hydrogen, halogen, C₁-C₄alkyl, C₁-C₄haloalkyl, C₁-C₄alkoxy, C₁-C₄haloalkoxy, more preferably hydrogen, fluorine, chlorine, C₁-C₂alkyl, C₁-C₂haloalkyl, most preferably hydrogen, fluorine, chlorine, methyl and trifluoromethyl.

Preferably R¹⁹ is selected from hydrogen, halogen, C₁-C₄alkyl, C₁-C₄haloalkyl, C₁-C₄alkoxy, C₁-C₄haloalkoxy, more preferably hydrogen, fluorine, chlorine, C₁-C₂alkyl, C₁-C₂haloalkyl, most preferably hydrogen, fluorine, chlorine, methyl, difluoromethyl, trifluoromethyl and 1,1-difluoroethyl.

In embodiments where two of X¹, X², X³, and X⁴ are nitrogen, preferably X² or X³ is nitrogen. In embodiments where one of X¹, X², X³, and X⁴ is nitrogen, preferably X² is nitrogen.

A preferred subset of compounds is one in which;
X¹ is C—R¹;
X² is nitrogen;
X³ is C—R¹⁸;
X⁴ is C—R¹⁹;
Y is C—H;
B is O;
n is 1 or 2;
R¹ is selected from hydrogen, fluorine, chlorine, C₁-C₂alkyl and C₁-C₂haloalkyl;
R² is selected from hydrogen, fluorine, chlorine, C₁-C₂alkyl and C₁-C₂haloalkyl;
R³ is selected from hydrogen, chlorine and fluorine;
R⁴ is selected from chlorine, bromine and cyano;
each R⁶ and R⁷ is independently selected from hydrogen, halogen and C₁-C₂alkyl;
R⁸ is OR⁹;
R⁹ is selected from hydrogen, C₁-C₄alkyl, C₁-C₂alkoxyC₁-C₂alkyl and phenylC₁-C₂alkyl;
R¹⁸ is selected from hydrogen, fluorine, chlorine, C₁-C₂alkyl and C₁-C₂haloalkyl;
R¹⁹ is selected from hydrogen, fluorine, chlorine, C₁-C₂alkyl and C₁-C₂haloalkyl.

Another preferred subset of compounds is one in which;
X¹ is C—R¹;
X² is C—R¹⁷;
X³ is nitrogen;
X⁴ is C—R¹⁹;
Y is C—H;
B is O;
n is 1 or 2;
R¹ is selected from hydrogen, fluorine, chlorine, C₁-C₂alkyl and C₁-C₂haloalkyl;
R² and R¹⁹ together with the carbon atoms to which they are attached form a 5- or 6-membered ring, which contains one or two heteroatoms selected from nitrogen, oxygen and sulfur and which is substituted with 1 to 4 groups represented by R¹⁶;
R³ is selected from hydrogen, chlorine and fluorine;
R⁴ is selected from chlorine, bromine and cyano;
each R⁶ and R⁷ is independently selected from hydrogen, halogen and C₁-C₂alkyl;
R⁸ is OR⁹;
R⁹ is selected from hydrogen, C₁-C₄alkyl, C₁-C₂alkoxyC₁-C₂alkyl and phenylC₁-C₂alkyl;
R¹⁶ is selected from halogen, C₁-C₄alkyl, C₁-C₄haloalkyl, C₁-C₄alkoxy, C₁-C₄haloalkoxy, cyano and C₁-C₄alkylsulfonyl;
R¹⁷ is selected from hydrogen, fluorine, chlorine, C₁-C₂alkyl and C₁-C₂haloalkyl.

A more preferred subset of compounds is one in which;
X¹ is C—R¹;
X² is nitrogen;
X³ is C—R¹⁸;
X⁴ is C—R¹⁹;
Y is C—H;
B is O;
n is 2;
R¹ is selected from hydrogen, fluorine, chlorine, methyl and trifluoromethyl;
R² is selected from hydrogen, fluorine, chlorine, methyl and trifluoromethyl;
R³ is selected from hydrogen and fluorine;
R⁴ is chlorine;
each R⁶ and R⁷ is independently selected from hydrogen, chlorine and methyl;
R⁸ is OR⁹;
R⁹ is selected from hydrogen, C₁-C₄alkyl and phenylC₁-C₂alkyl;
R¹⁸ is selected from hydrogen, fluorine, chlorine, methyl and trifluoromethyl;
R¹⁹ is selected from hydrogen, fluorine, chlorine, methyl, difluoromethyl, trifluoromethyl and 1,1-difluoroethyl.

Another more preferred subset of compounds is one in which;
X¹ is C—R¹;
X² is C—R¹⁷;
X³ is nitrogen;
X⁴ is C—R¹⁹;
Y is C—H;
B is O;
n is 2;
R¹ is selected from hydrogen, fluorine, chlorine, methyl and trifluoromethyl;
R² and R¹⁹ together with the carbon atoms to which they are attached form a saturated 5-membered ring, which contains one or two oxygen atoms and which is substituted with 1 to 3 groups represented by R¹⁶;
R³ is selected from hydrogen and fluorine;
R⁴ is chlorine;
each R⁶ and R⁷ is independently selected from hydrogen, chlorine and methyl;
R⁸ is OR⁹;
R⁹ is selected from hydrogen, C₁-C₄alkyl and phenylC₁-C₂alkyl;
R¹⁶ is halogen;
R¹⁷ is selected from hydrogen, fluorine, chlorine, methyl and trifluoromethyl.

In one embodiment, there is provided a compound of formula (I), wherein:
X¹ is C—Cl;
X² is nitrogen;
X³ is C—H;
X⁴ is C—CF₃;
Y is C—H or nitrogen;
R² is hydrogen;
R³ is hydrogen or fluorine;
R⁴ is chlorine;
R⁶ and R⁷ are each independently selected from hydrogen and methyl;
R⁸ is OR⁹; and
R⁹ is C₁-C₂alkyl.

### Tables of Examples

Table 1 below discloses 1140 specific compounds of formula (I), designated compounds 1-1 to 1-1140 respectively, wherein R³ is hydrogen, R⁴ is chlorine, Y is C—H, and B is oxygen.

TABLE 1

| Compound | | | | | | | |
|---|---|---|---|---|---|---|---|
| Number | X¹ | X² | X³ | X⁴ | R² | D | R⁸ |
| 1-1 | CCl | CH | CH | CMe | H | CH₂ | OH |
| 1-2 | CCl | CH | CH | CMe | H | CH₂ | OMe |
| 1-3 | CCl | CH | CH | CMe | H | CH₂ | OEt |
| 1-4 | CCl | CH | CH | CMe | H | CH₂ | OCH₂Ph |
| 1-5 | CCl | CH | CH | CMe | H | CH₂ | NHSO₂Me |
| 1-6 | CCl | CH | CH | CMe | H | CH₂ | NHSO₂N(Me)(CHMe₂) |
| 1-7 | N | CH | N | CCF₃ | H | CH₂ | OH |
| 1-8 | N | CH | N | CCF₃ | H | CH₂ | OMe |
| 1-9 | N | CH | N | CCF₃ | H | CH₂ | OEt |
| 1-10 | N | CH | N | CCF₃ | H | CH₂ | OCH₂Ph |
| 1-11 | N | CH | N | CCF₃ | H | CH₂ | NHSO₂Me |
| 1-12 | N | CH | N | CCF₃ | H | CH₂ | NHSO₂N(Me)(CHMe₂) |
| 1-13 | NO | CCl | CH | CCF₃ | H | CH₂ | OH |
| 1-14 | NO | CCl | CH | CCF₃ | H | CH₂ | OMe |
| 1-15 | NO | CCl | CH | CCF₃ | H | CH₂ | OEt |
| 1-16 | NO | CCl | CH | CCF₃ | H | CH₂ | OCH₂Ph |
| 1-17 | NO | CCl | CH | CCF₃ | H | CH₂ | NHSO₂Me |
| 1-18 | NO | CCl | CH | CCF₃ | H | CH₂ | NHSO₂N(Me)(CHMe₂) |
| 1-19 | CF | N | CH | CF | H | CH₂ | OH |
| 1-20 | CF | N | CH | CF | H | CH₂ | OMe |
| 1-21 | CF | N | CH | CF | H | CH₂ | OEt |
| 1-22 | CF | N | CH | CF | H | CH₂ | OCH₂Ph |
| 1-23 | CF | N | CH | CF | H | CH₂ | NHSO₂Me |
| 1-24 | CF | N | CH | CF | H | CH₂ | NHSO₂N(Me)(CHMe₂) |
| 1-25 | CCl | N | CH | CF | H | CH₂ | OH |
| 1-26 | CCl | N | CH | CF | H | CH₂ | OMe |
| 1-27 | CCl | N | CH | CF | H | CH₂ | OEt |
| 1-28 | CCl | N | CH | CF | H | CH₂ | OCH₂Ph |
| 1-29 | CCl | N | CH | CF | H | CH₂ | NHSO₂Me |
| 1-30 | CCl | N | CH | CF | H | CH₂ | NHSO₂N(Me)(CHMe₂) |
| 1-31 | CCl | N | CH | CCl | H | CH₂ | OH |
| 1-32 | CCl | N | CH | CCl | H | CH₂ | OMe |
| 1-33 | CCl | N | CH | CCl | H | CH₂ | OEt |
| 1-34 | CCl | N | CH | CCl | H | CH₂ | OCH₂Ph |
| 1-35 | CCl | N | CH | CCl | H | CH₂ | NHSO₂Me |
| 1-36 | CCl | N | CH | CCl | H | CH₂ | NHSO₂N(Me)(CHMe₂) |
| 1-37 | CCF₃ | N | CH | CCl | H | CH₂ | OH |
| 1-38 | CCF₃ | N | CH | CCl | H | CH₂ | OMe |
| 1-39 | CCF₃ | N | CH | CCl | H | CH₂ | OEt |
| 1-40 | CCF₃ | N | CH | CCl | H | CH₂ | OCH₂Ph |
| 1-41 | CCF₃ | N | CH | CCl | H | CH₂ | NHSO₂Me |
| 1-42 | CCF₃ | N | CH | CCl | H | CH₂ | NHSO₂N(Me)(CHMe₂) |
| 1-43 | CCl | N | CH | CBr | H | CH₂ | OH |
| 1-44 | CCl | N | CH | CBr | H | CH₂ | OMe |
| 1-45 | CCl | N | CH | CBr | H | CH₂ | OEt |
| 1-46 | CCl | N | CH | CBr | H | CH₂ | OCH₂Ph |
| 1-47 | CCl | N | CH | CBr | H | CH₂ | NHSO₂Me |
| 1-48 | CCl | N | CH | CBr | H | CH₂ | NHSO₂N(Me)(CHMe₂) |
| 1-49 | CCl | N | CH | CMe | H | CH₂ | OH |
| 1-50 | CCl | N | CH | CMe | H | CH₂ | OMe |
| 1-51 | CCl | N | CH | CMe | H | CH₂ | OEt |
| 1-52 | CCl | N | CH | CMe | H | CH₂ | OCH₂Ph |
| 1-53 | CCl | N | CH | CMe | H | CH₂ | NHSO₂Me |
| 1-54 | CCl | N | CH | CMe | H | CH₂ | NHSO₂N(Me)(CHMe₂) |
| 1-55 | CCl | N | CH | CCF₂H | H | CH₂ | OH |
| 1-56 | CCl | N | CH | CCF₂H | H | CH₂ | OMe |
| 1-57 | CCl | N | CH | CCF₂H | H | CH₂ | OEt |
| 1-58 | CCl | N | CH | CCF₂H | H | CH₂ | OCH₂Ph |
| 1-59 | CCl | N | CH | CCF₂H | H | CH₂ | NHSO₂Me |
| 1-60 | CCl | N | CH | CCF₂H | H | CH₂ | NHSO₂N(Me)(CHMe₂) |
| 1-61 | CCl | N | CH | CCF₂Me | H | CH₂ | OH |
| 1-62 | CCl | N | CH | CCF₂Me | H | CH₂ | OMe |
| 1-63 | CCl | N | CH | CCF₂Me | H | CH₂ | OEt |
| 1-64 | CCl | N | CH | CCF₂Me | H | CH₂ | OCH₂Ph |
| 1-65 | CCl | N | CH | CCF₂Me | H | CH₂ | NHSO₂Me |
| 1-66 | CCl | N | CH | CCF₂Me | H | CH₂ | NHSO₂N(Me)(CHMe₂) |
| 1-67 | CCl | N | CH | CCF₃ | H | CH₂ | OH |
| 1-68 | CCl | N | CH | CCF₃ | H | CH₂ | OMe |
| 1-69 | CCl | N | CH | CCF₃ | H | CH₂ | OEt |
| 1-70 | CCl | N | CH | CCF₃ | H | CH₂ | OCH₂Ph |
| 1-71 | CCl | N | CH | CCF₃ | H | CH₂ | NHSO₂Me |
| 1-72 | CCl | N | CH | CCF₃ | H | CH₂ | NHSO₂N(Me)(CHMe₂) |
| 1-73 | CH | N | CH | CCF₃ | H | CH₂ | OH |
| 1-74 | CH | N | CH | CCF₃ | H | CH₂ | OMe |
| 1-75 | CH | N | CH | CCF₃ | H | CH₂ | OEt |
| 1-76 | CH | N | CH | CCF₃ | H | CH₂ | OCH₂Ph |
| 1-77 | CH | N | CH | CCF₃ | H | CH₂ | NHSO₂Me |
| 1-78 | CH | N | CH | CCF₃ | H | CH₂ | NHSO₂N(Me)(CHMe₂) |
| 1-79 | CF | N | CH | CCF₃ | H | CH₂ | OH |
| 1-80 | CF | N | CH | CCF₃ | H | CH₂ | OMe |
| 1-81 | CF | N | CH | CCF₃ | H | CH₂ | OEt |
| 1-82 | CF | N | CH | CCF₃ | H | CH₂ | OCH₂Ph |
| 1-83 | CF | N | CH | CCF₃ | H | CH₂ | NHSO₂Me |
| 1-84 | CF | N | CH | CCF₃ | H | CH₂ | NHSO₂N(Me)(CHMe₂) |
| 1-85 | CBr | N | CH | CCF₃ | H | CH₂ | OH |
| 1-86 | CBr | N | CH | CCF₃ | H | CH₂ | OMe |
| 1-87 | CBr | N | CH | CCF₃ | H | CH₂ | OEt |
| 1-88 | CBr | N | CH | CCF₃ | H | CH₂ | OCH₂Ph |
| 1-89 | CBr | N | CH | CCF₃ | H | CH₂ | NHSO₂Me |
| 1-90 | CBr | N | CH | CCF₃ | H | CH₂ | NHSO₂N(Me)(CHMe₂) |
| 1-91 | CCl | N | CH | CSO₂Me | H | CH₂ | OH |
| 1-92 | CCl | N | CH | CSO₂Me | H | CH₂ | OMe |
| 1-93 | CCl | N | CH | CSO₂Me | H | CH₂ | OEt |
| 1-94 | CCl | N | CH | CSO₂Me | H | CH₂ | OCH₂Ph |
| 1-95 | CCl | N | CH | CSO₂Me | H | CH₂ | NHSO₂Me |
| 1-96 | CCl | N | CH | CSO₂Me | H | CH₂ | NHSO₂N(Me)(CHMe₂) |
| 1-97 | CH | CH | N | COCF₂O | CH₂ | OH | |
| 1-98 | CH | CH | N | COCF₂O | CH₂ | OMe | |
| 1-99 | CH | CH | N | COCF₂O | CH₂ | OEt | |
| 1-100 | CH | CH | N | COCF₂O | CH₂ | OCH₂Ph | |
| 1-101 | CH | CH | N | COCF₂O | CH₂ | NHSO₂Me | |
| 1-102 | CH | CH | N | COCF₂O | CH₂ | NHSO₂N(Me)(CHMe₂) | |
| 1-103 | CH | CF | N | CH | CF₃ | CH₂ | OH |
| 1-104 | CH | CF | N | CH | CF₃ | CH₂ | OMe |
| 1-105 | CH | CF | N | CH | CF₃ | CH₂ | OEt |
| 1-106 | CH | CF | N | CH | CF₃ | CH₂ | OCH₂Ph |
| 1-107 | CH | CF | N | CH | CF₃ | CH₂ | NHSO₂Me |
| 1-108 | CH | CF | N | CH | CF₃ | CH₂ | NHSO₂N(Me)(CHMe₂) |
| 1-109 | CH | CCl | CCl | N | H | CH₂ | OH |
| 1-110 | CH | CCl | CCl | N | H | CH₂ | OMe |
| 1-111 | CH | CCl | CCl | N | H | CH₂ | OEt |
| 1-112 | CH | CCl | CCl | N | H | CH₂ | OCH₂Ph |
| 1-113 | CH | CCl | CCl | N | H | CH₂ | NHSO₂Me |
| 1-114 | CH | CCl | CCl | N | H | CH₂ | NHSO₂N(Me)(CHMe₂) |
| 1-115 | CCl | CH | CH | CMe | H | CHMe | OH |
| 1-116 | CCl | CH | CH | CMe | H | CHMe | OMe |
| 1-117 | CCl | CH | CH | CMe | H | CHMe | OEt |
| 1-118 | CCl | CH | CH | CMe | H | CHMe | OCH₂Ph |
| 1-119 | CCl | CH | CH | CMe | H | CHMe | NHSO₂Me |
| 1-120 | CCl | CH | CH | CMe | H | CHMe | NHSO₂N(Me)(CHMe₂) |
| 1-121 | N | CH | N | CCF₃ | H | CHMe | OH |
| 1-122 | N | CH | N | CCF₃ | H | CHMe | OMe |
| 1-123 | N | CH | N | CCF₃ | H | CHMe | OEt |
| 1-124 | N | CH | N | CCF₃ | H | CHMe | OCH₂Ph |
| 1-125 | N | CH | N | CCF₃ | H | CHMe | NHSO₂Me |
| 1-126 | N | CH | N | CCF₃ | H | CHMe | NHSO₂N(Me)(CHMe₂) |
| 1-127 | NO | CCl | CH | CCF₃ | H | CHMe | OH |
| 1-128 | NO | CCl | CH | CCF₃ | H | CHMe | OMe |
| 1-129 | NO | CCl | CH | CCF₃ | H | CHMe | OEt |
| 1-130 | NO | CCl | CH | CCF₃ | H | CHMe | OCH₂Ph |
| 1-131 | NO | CCl | CH | CCF₃ | H | CHMe | NHSO₂Me |
| 1-132 | NO | CCl | CH | CCF₃ | H | CHMe | NHSO₂N(Me)(CHMe₂) |
| 1-133 | CF | N | CH | CF | H | CHMe | OH |
| 1-134 | CF | N | CH | CF | H | CHMe | OMe |
| 1-135 | CF | N | CH | CF | H | CHMe | OEt |
| 1-136 | CF | N | CH | CF | H | CHMe | OCH₂Ph |
| 1-137 | CF | N | CH | CF | H | CHMe | NHSO₂Me |
| 1-138 | CF | N | CH | CF | H | CHMe | NHSO₂N(Me)(CHMe₂) |
| 1-139 | CCl | N | CH | CF | H | CHMe | OH |
| 1-140 | CCl | N | CH | CF | H | CHMe | OMe |
| 1-141 | CCl | N | CH | CF | H | CHMe | OEt |
| 1-142 | CCl | N | CH | CF | H | CHMe | OCH₂Ph |
| 1-143 | CCl | N | CH | CF | H | CHMe | NHSO₂Me |
| 1-144 | CCl | N | CH | CF | H | CHMe | NHSO₂N(Me)(CHMe₂) |
| 1-145 | CCl | N | CH | CCl | H | CHMe | OH |
| 1-146 | CCl | N | CH | CCl | H | CHMe | OMe |
| 1-147 | CCl | N | CH | CCl | H | CHMe | OEt |
| 1-148 | CCl | N | CH | CCl | H | CHMe | OCH₂Ph |
| 1-149 | CCl | N | CH | CCl | H | CHMe | NHSO₂Me |
| 1-150 | CCl | N | CH | CCl | H | CHMe | NHSO₂N(Me)(CHMe₂) |
| 1-151 | CCF₃ | N | CH | CCl | H | CHMe | OH |
| 1-152 | CCF₃ | N | CH | CCl | H | CHMe | OMe |
| 1-153 | CCF₃ | N | CH | CCl | H | CHMe | OEt |
| 1-154 | CCF₃ | N | CH | CCl | H | CHMe | OCH₂Ph |
| 1-155 | CCF₃ | N | CH | CCl | H | CHMe | NHSO₂Me |
| 1-156 | CCF₃ | N | CH | CCl | H | CHMe | NHSO₂N(Me)(CHMe₂) |
| 1-157 | CCl | N | CH | CBr | H | CHMe | OH |
| 1-158 | CCl | N | CH | CBr | H | CHMe | OMe |
| 1-159 | CCl | N | CH | CBr | H | CHMe | OEt |
| 1-160 | CCl | N | CH | CBr | H | CHMe | OCH₂Ph |
| 1-161 | CCl | N | CH | CBr | H | CHMe | NHSO₂Me |
| 1-162 | CCl | N | CH | CBr | H | CHMe | NHSO₂N(Me)(CHMe₂) |
| 1-163 | CCl | N | CH | CMe | H | CHMe | OH |
| 1-164 | CCl | N | CH | CMe | H | CHMe | OMe |
| 1-165 | CCl | N | CH | CMe | H | CHMe | OEt |
| 1-166 | CCl | N | CH | CMe | H | CHMe | OCH₂Ph |
| 1-167 | CCl | N | CH | CMe | H | CHMe | NHSO₂Me |
| 1-168 | CCl | N | CH | CMe | H | CHMe | NHSO₂N(Me)(CHMe₂) |
| 1-169 | CCl | N | CH | CCF₂H | H | CHMe | OH |
| 1-170 | CCl | N | CH | CCF₂H | H | CHMe | OMe |
| 1-171 | CCl | N | CH | CCF₂H | H | CHMe | OEt |
| 1-172 | CCl | N | CH | CCF₂H | H | CHMe | OCH₂Ph |
| 1-173 | CCl | N | CH | CCF₂H | H | CHMe | NHSO₂Me |
| 1-174 | CCl | N | CH | CCF₂H | H | CHMe | NHSO₂N(Me)(CHMe₂) |
| 1-175 | CCl | N | CH | CCF₂Me | H | CHMe | OH |
| 1-176 | CCl | N | CH | CCF₂Me | H | CHMe | OMe |
| 1-177 | CCl | N | CH | CCF₂Me | H | CHMe | OEt |
| 1-178 | CCl | N | CH | CCF₂Me | H | CHMe | OCH₂Ph |
| 1-179 | CCl | N | CH | CCF₂Me | H | CHMe | NHSO₂Me |
| 1-180 | CCl | N | CH | CCF₂Me | H | CHMe | NHSO₂N(Me)(CHMe₂) |
| 1-181 | CCl | N | CH | CCF₃ | H | CHMe | OH |
| 1-182 | CCl | N | CH | CCF₃ | H | CHMe | OMe |
| 1-183 | CCl | N | CH | CCF₃ | H | CHMe | OEt |
| 1-184 | CCl | N | CH | CCF₃ | H | CHMe | OCH₂Ph |
| 1-185 | CCl | N | CH | CCF₃ | H | CHMe | NHSO₂Me |
| 1-186 | CCl | N | CH | CCF₃ | H | CHMe | NHSO₂N(Me)(CHMe₂) |
| 1-187 | CH | N | CH | CCF₃ | H | CHMe | OH |
| 1-188 | CH | N | CH | CCF₃ | H | CHMe | OMe |
| 1-189 | CH | N | CH | CCF₃ | H | CHMe | OEt |
| 1-190 | CH | N | CH | CCF₃ | H | CHMe | OCH₂Ph |
| 1-191 | CH | N | CH | CCF₃ | H | CHMe | NHSO₂Me |
| 1-192 | CH | N | CH | CCF₃ | H | CHMe | NHSO₂N(Me)(CHMe₂) |
| 1-193 | CF | N | CH | CCF₃ | H | CHMe | OH |
| 1-194 | CF | N | CH | CCF₃ | H | CHMe | OMe |
| 1-195 | CF | N | CH | CCF₃ | H | CHMe | OEt |
| 1-196 | CF | N | CH | CCF₃ | H | CHMe | OCH₂Ph |
| 1-197 | CF | N | CH | CCF₃ | H | CHMe | NHSO₂Me |
| 1-198 | CF | N | CH | CCF₃ | H | CHMe | NHSO₂N(Me)(CHMe₂) |
| 1-199 | CBr | N | CH | CCF₃ | H | CHMe | OH |
| 1-200 | CBr | N | CH | CCF₃ | H | CHMe | OMe |
| 1-201 | CBr | N | CH | CCF₃ | H | CHMe | OEt |
| 1-202 | CBr | N | CH | CCF₃ | H | CHMe | OCH₂Ph |
| 1-203 | CBr | N | CH | CCF₃ | H | CHMe | NHSO₂Me |
| 1-204 | CBr | N | CH | CCF₃ | H | CHMe | NHSO₂N(Me)(CHMe₂) |
| 1-205 | CCl | N | CH | CSO₂Me | H | CHMe | OH |
| 1-206 | CCl | N | CH | CSO₂Me | H | CHMe | OMe |
| 1-207 | CCl | N | CH | CSO₂Me | H | CHMe | OEt |
| 1-208 | CCl | N | CH | CSO₂Me | H | CHMe | OCH₂Ph |
| 1-209 | CCl | N | CH | CSO₂Me | H | CHMe | NHSO₂Me |
| 1-210 | CCl | N | CH | CSO₂Me | H | CHMe | NHSO₂N(Me)(CHMe₂) |
| 1-211 | CH | CH | N | COCF₂O | | CHMe | OH |
| 1-212 | CH | CH | N | COCF₂O | | CHMe | OMe |
| 1-213 | CH | CH | N | COCF₂O | | CHMe | OEt |
| 1-214 | CH | CH | N | COCF₂O | | CHMe | OCH₂Ph |
| 1-215 | CH | CH | N | COCF₂O | | CHMe | NHSO₂Me |
| 1-216 | CH | CH | N | COCF₂O | | CHMe | NHSO₂N(Me)(CHMe₂) |
| 1-217 | CH | CF | N | CH | CF₃ | CHMe | OH |
| 1-218 | CH | CF | N | CH | CF₃ | CHMe | OMe |
| 1-219 | CH | CF | N | CH | CF₃ | CHMe | OEt |
| 1-220 | CH | CF | N | CH | CF₃ | CHMe | OCH₂Ph |
| 1-221 | CH | CF | N | CH | CF₃ | CHMe | NHSO₂Me |
| 1-222 | CH | CF | N | CH | CF₃ | CHMe | NHSO₂N(Me)(CHMe₂) |
| 1-223 | CH | CCl | CCl | N | H | CHMe | OH |
| 1-224 | CH | CCl | CCl | N | H | CHMe | OMe |
| 1-225 | CH | CCl | CCl | N | H | CHMe | OEt |
| 1-226 | CH | CCl | CCl | N | H | CHMe | OCH₂Ph |
| 1-227 | CH | CCl | CCl | N | H | CHMe | NHSO₂Me |
| 1-228 | CH | CCl | CCl | N | H | CHMe | NHSO₂N(Me)(CHMe₂) |
| 1-229 | CCl | CH | CH | CMe | H | CMe₂ | OH |
| 1-230 | CCl | CH | CH | CMe | H | CMe₂ | OMe |
| 1-231 | CCl | CH | CH | CMe | H | CMe₂ | OEt |
| 1-232 | CCl | CH | CH | CMe | H | CMe₂ | OCH₂Ph |
| 1-233 | CCl | CH | CH | CMe | H | CMe₂ | NHSO₂Me |
| 1-234 | CCl | CH | CH | CMe | H | CMe₂ | NHSO₂N(Me)(CHMe₂) |
| 1-235 | N | CH | N | CCF₃ | H | CMe₂ | OH |
| 1-236 | N | CH | N | CCF₃ | H | CMe₂ | OMe |
| 1-237 | N | CH | N | CCF₃ | H | CMe₂ | OEt |
| 1-238 | N | CH | N | CCF₃ | H | CMe₂ | OCH₂Ph |
| 1-239 | N | CH | N | CCF₃ | H | CMe₂ | NHSO₂Me |
| 1-240 | N | CH | N | CCF₃ | H | CMe₂ | NHSO₂N(Me)(CHMe₂) |
| 1-241 | NO | CCl | CH | CCF₃ | H | CMe₂ | OH |
| 1-242 | NO | CCl | CH | CCF₃ | H | CMe₂ | OMe |
| 1-243 | NO | CCl | CH | CCF₃ | H | CMe₂ | OEt |
| 1-244 | NO | CCl | CH | CCF₃ | H | CMe₂ | OCH₂Ph |
| 1-245 | NO | CCl | CH | CCF₃ | H | CMe₂ | NHSO₂Me |
| 1-246 | NO | CCl | CH | CCF₃ | H | CMe₂ | NHSO₂N(Me)(CHMe₂) |
| 1-247 | CF | N | CH | CF | H | CMe₂ | OH |
| 1-248 | CF | N | CH | CF | H | CMe₂ | OMe |
| 1-249 | CF | N | CH | CF | H | CMe₂ | OEt |
| 1-250 | CF | N | CH | CF | H | CMe₂ | OCH₂Ph |
| 1-251 | CF | N | CH | CF | H | CMe₂ | NHSO₂Me |
| 1-252 | CF | N | CH | CF | H | CMe₂ | NHSO₂N(Me)(CHMe₂) |
| 1-253 | CCl | N | CH | CF | H | CMe₂ | OH |
| 1-254 | CCl | N | CH | CF | H | CMe₂ | OMe |
| 1-255 | CCl | N | CH | CF | H | CMe₂ | OEt |
| 1-256 | CCl | N | CH | CF | H | CMe₂ | OCH₂Ph |
| 1-257 | CCl | N | CH | CF | H | CMe₂ | NHSO₂Me |
| 1-258 | CCl | N | CH | CF | H | CMe₂ | NHSO₂N(Me)(CHMe₂) |
| 1-259 | CCl | N | CH | CCl | H | CMe₂ | OH |
| 1-260 | CCl | N | CH | CCl | H | CMe₂ | OMe |
| 1-261 | CCl | N | CH | CCl | H | CMe₂ | OEt |
| 1-262 | CCl | N | CH | CCl | H | CMe₂ | OCH₂Ph |
| 1-263 | CCl | N | CH | CCl | H | CMe₂ | NHSO₂Me |
| 1-264 | CCl | N | CH | CCl | H | CMe₂ | NHSO₂N(Me)(CHMe₂) |
| 1-265 | CCF₃ | N | CH | CCl | H | CMe₂ | OH |
| 1-266 | CCF₃ | N | CH | CCl | H | CMe₂ | OMe |
| 1-267 | CCF₃ | N | CH | CCl | H | CMe₂ | OEt |
| 1-268 | CCF₃ | N | CH | CCl | H | CMe₂ | OCH₂Ph |
| 1-269 | CCF₃ | N | CH | CCl | H | CMe₂ | NHSO₂Me |
| 1-270 | CCF₃ | N | CH | CCl | H | CMe₂ | NHSO₂N(Me)(CHMe₂) |
| 1-271 | CCl | N | CH | CBr | H | CMe₂ | OH |
| 1-272 | CCl | N | CH | CBr | H | CMe₂ | OMe |
| 1-273 | CCl | N | CH | CBr | H | CMe₂ | OEt |
| 1-274 | CCl | N | CH | CBr | H | CMe₂ | OCH₂Ph |
| 1-275 | CCl | N | CH | CBr | H | CMe₂ | NHSO₂Me |
| 1-276 | CCl | N | CH | CBr | H | CMe₂ | NHSO₂N(Me)(CHMe₂) |
| 1-277 | CCl | N | CH | CMe | H | CMe₂ | OH |
| 1-278 | CCl | N | CH | CMe | H | CMe₂ | OMe |
| 1-279 | CCl | N | CH | CMe | H | CMe₂ | OEt |
| 1-280 | CCl | N | CH | CMe | H | CMe₂ | OCH₂Ph |
| 1-281 | CCl | N | CH | CMe | H | CMe₂ | NHSO₂Me |
| 1-282 | CCl | N | CH | CMe | H | CMe₂ | NHSO₂N(Me)(CHMe₂) |
| 1-283 | CCl | N | CH | CCF₂H | H | CMe₂ | OH |
| 1-284 | CCl | N | CH | CCF₂H | H | CMe₂ | OMe |
| 1-285 | CCl | N | CH | CCF₂H | H | CMe₂ | OEt |
| 1-286 | CCl | N | CH | CCF₂H | H | CMe₂ | OCH₂Ph |
| 1-287 | CCl | N | CH | CCF₂H | H | CMe₂ | NHSO₂Me |
| 1-288 | CCl | N | CH | CCF₂H | H | CMe₂ | NHSO₂N(Me)(CHMe₂) |
| 1-289 | CCl | N | CH | CCF₂Me | H | CMe₂ | OH |
| 1-290 | CCl | N | CH | CCF₂Me | H | CMe₂ | OMe |
| 1-291 | CCl | N | CH | CCF₂Me | H | CMe₂ | OEt |
| 1-292 | CCl | N | CH | CCF₂Me | H | CMe₂ | OCH₂Ph |
| 1-293 | CCl | N | CH | CCF₂Me | H | CMe₂ | NHSO₂Me |
| 1-294 | CCl | N | CH | CCF₂Me | H | CMe₂ | NHSO₂N(Me)(CHMe₂) |
| 1-295 | CCl | N | CH | CCF₃ | H | CMe₂ | OH |
| 1-296 | CCl | N | CH | CCF₃ | H | CMe₂ | OMe |
| 1-297 | CCl | N | CH | CCF₃ | H | CMe₂ | OEt |
| 1-298 | CCl | N | CH | CCF₃ | H | CMe₂ | OCH₂Ph |
| 1-299 | CCl | N | CH | CCF₃ | H | CMe₂ | NHSO₂Me |
| 1-300 | CCl | N | CH | CCF₃ | H | CMe₂ | NHSO₂N(Me)(CHMe₂) |
| 1-301 | CH | N | CH | CCF₃ | H | CMe₂ | OH |
| 1-302 | CH | N | CH | CCF₃ | H | CMe₂ | OMe |
| 1-303 | CH | N | CH | CCF₃ | H | CMe₂ | OEt |
| 1-304 | CH | N | CH | CCF₃ | H | CMe₂ | OCH₂Ph |
| 1-305 | CH | N | CH | CCF₃ | H | CMe₂ | NHSO₂Me |
| 1-306 | CH | N | CH | CCF₃ | H | CMe₂ | NHSO₂N(Me)(CHMe₂) |
| 1-307 | CF | N | CH | CCF₃ | H | CMe₂ | OH |
| 1-308 | CF | N | CH | CCF₃ | H | CMe₂ | OMe |
| 1-309 | CF | N | CH | CCF₃ | H | CMe₂ | OEt |
| 1-310 | CF | N | CH | CCF₃ | H | CMe₂ | OCH₂Ph |
| 1-311 | CF | N | CH | CCF₃ | H | CMe₂ | NHSO₂Me |
| 1-312 | CF | N | CH | CCF₃ | H | CMe₂ | NHSO₂N(Me)(CHMe₂) |
| 1-313 | CBr | N | CH | CCF₃ | H | CMe₂ | OH |
| 1-314 | CBr | N | CH | CCF₃ | H | CMe₂ | OMe |
| 1-315 | CBr | N | CH | CCF₃ | H | CMe₂ | OEt |
| 1-316 | CBr | N | CH | CCF₃ | H | CMe₂ | OCH₂Ph |
| 1-317 | CBr | N | CH | CCF₃ | H | CMe₂ | NHSO₂Me |
| 1-318 | CBr | N | CH | CCF₃ | H | CMe₂ | NHSO₂N(Me)(CHMe₂) |
| 1-319 | CCl | N | CH | CSO₂Me | H | CMe₂ | OH |
| 1-320 | CCl | N | CH | CSO₂Me | H | CMe₂ | OMe |
| 1-321 | CCl | N | CH | CSO₂Me | H | CMe₂ | OEt |
| 1-322 | CCl | N | CH | CSO₂Me | H | CMe₂ | OCH₂Ph |
| 1-323 | CCl | N | CH | CSO₂Me | H | CMe₂ | NHSO₂Me |
| 1-324 | CCl | N | CH | CSO₂Me | H | CMe₂ | NHSO₂N(Me)(CHMe₂) |
| 1-325 | CH | CH | N | COCF₂O | CMe₂ | OH | |
| 1-326 | CH | CH | N | COCF₂O | CMe₂ | OMe | |
| 1-327 | CH | CH | N | COCF₂O | CMe₂ | OEt | |
| 1-328 | CH | CH | N | COCF₂O | CMe₂ | OCH₂Ph | |
| 1-329 | CH | CH | N | COCF₂O | CMe₂ | NHSO₂Me | |
| 1-330 | CH | CH | N | COCF₂O | CMe₂ | NHSO₂N(Me)(CHMe₂) | |
| 1-331 | CH | F | N | CH | CF₃ | CMe₂ | OH |
| 1-332 | CH | F | N | CH | CF₃ | CMe₂ | OMe |
| 1-333 | CH | F | N | CH | CF₃ | CMe₂ | OEt |
| 1-334 | CH | F | N | CH | CF₃ | CMe₂ | OCH₂Ph |
| 1-335 | CH | F | N | CH | CF₃ | CMe₂ | NHSO₂Me |
| 1-336 | CH | F | N | CH | CF₃ | CMe₂ | NHSO₂N(Me)(CHMe₂) |
| 1-337 | CH | CCl | CCl | N | H | CMe₂ | OH |
| 1-338 | CH | CCl | CCl | N | H | CMe₂ | OMe |
| 1-339 | CH | CCl | CCl | N | H | CMe₂ | OEt |
| 1-340 | CH | CCl | CCl | N | H | CMe₂ | OCH₂Ph |
| 1-341 | CH | CCl | CCl | N | H | CMe₂ | NHSO₂Me |
| 1-342 | CH | CCl | CCl | N | H | CMe₂ | NHSO₂N(Me)(CHMe₂) |
| 1-343 | CCl | CH | CH | CMe | H | CH₂CH₂ | OH |
| 1-344 | CCl | CH | CH | CMe | H | CH₂CH₂ | OMe |
| 1-345 | CCl | CH | CH | CMe | H | CH₂CH₂ | OEt |
| 1-346 | CCl | CH | CH | CMe | H | CH₂CH₂ | OCH₂Ph |
| 1-347 | CCl | CH | CH | CMe | H | CH₂CH₂ | NHSO₂Me |
| 1-348 | CCl | CH | CH | CMe | H | CH₂CH₂ | NHSO₂N(Me)(CHMe₂) |
| 1-349 | N | CH | N | CCF₃ | H | CH₂CH₂ | OH |
| 1-350 | N | CH | N | CCF₃ | H | CH₂CH₂ | OMe |
| 1-351 | N | CH | N | CCF₃ | H | CH₂CH₂ | OEt |
| 1-352 | N | CH | N | CCF₃ | H | CH₂CH₂ | OCH₂Ph |
| 1-353 | N | CH | N | CCF₃ | H | CH₂CH₂ | NHSO₂Me |
| 1-354 | N | CH | N | CCF₃ | H | CH₂CH₂ | NHSO₂N(Me)(CHMe₂) |
| 1-355 | NO | CCl | CH | CCF₃ | H | CH₂CH₂ | OH |
| 1-356 | NO | CCl | CH | CCF₃ | H | CH₂CH₂ | OMe |
| 1-357 | NO | CCl | CH | CCF₃ | H | CH₂CH₂ | OEt |
| 1-358 | NO | CCl | CH | CCF₃ | H | CH₂CH₂ | OCH₂Ph |
| 1-359 | NO | CCl | CH | CCF₃ | H | CH₂CH₂ | NHSO₂Me |
| 1-360 | NO | CCl | CH | CCF₃ | H | CH₂CH₂ | NHSO₂N(Me)(CHMe₂) |
| 1-361 | CF | N | CH | CF | H | CH₂CH₂ | OH |
| 1-362 | CF | N | CH | CF | H | CH₂CH₂ | OMe |
| 1-363 | CF | N | CH | CF | H | CH₂CH₂ | OEt |
| 1-364 | CF | N | CH | CF | H | CH₂CH₂ | OCH₂Ph |
| 1-365 | CF | N | CH | CF | H | CH₂CH₂ | NHSO₂Me |
| 1-366 | CF | N | CH | CF | H | CH₂CH₂ | NHSO₂N(Me)(CHMe₂) |
| 1-367 | CCl | N | CH | CF | H | CH₂CH₂ | OH |
| 1-368 | CCl | N | CH | CF | H | CH₂CH₂ | OMe |
| 1-369 | CCl | N | CH | CF | H | CH₂CH₂ | OEt |
| 1-370 | CCl | N | CH | CF | H | CH₂CH₂ | OCH₂Ph |
| 1-371 | CCl | N | CH | CF | H | CH₂CH₂ | NHSO₂Me |
| 1-372 | CCl | N | CH | CF | H | CH₂CH₂ | NHSO₂N(Me)(CHMe₂) |
| 1-373 | CCl | N | CH | CCl | H | CH₂CH₂ | OH |
| 1-374 | CCl | N | CH | CCl | H | CH₂CH₂ | OMe |
| 1-375 | CCl | N | CH | CCl | H | CH₂CH₂ | OEt |
| 1-376 | CCl | N | CH | CCl | H | CH₂CH₂ | OCH₂Ph |
| 1-377 | CCl | N | CH | CCl | H | CH₂CH₂ | NHSO₂Me |
| 1-378 | CCl | N | CH | CCl | H | CH₂CH₂ | NHSO₂N(Me)(CHMe₂) |
| 1-379 | CCF₃ | N | CH | CCl | H | CH₂CH₂ | OH |
| 1-380 | CCF₃ | N | CH | CCl | H | CH₂CH₂ | OMe |
| 1-381 | CCF₃ | N | CH | CCl | H | CH₂CH₂ | OEt |
| 1-382 | CCF₃ | N | CH | CCl | H | CH₂CH₂ | OCH₂Ph |
| 1-383 | CCF₃ | N | CH | CCl | H | CH₂CH₂ | NHSO₂Me |
| 1-384 | CCF₃ | N | CH | CCl | H | CH₂CH₂ | NHSO₂N(Me)(CHMe₂) |
| 1-385 | CCl | N | CH | CBr | H | CH₂CH₂ | OH |
| 1-386 | CCl | N | CH | CBr | H | CH₂CH₂ | OMe |
| 1-387 | CCl | N | CH | CBr | H | CH₂CH₂ | OEt |
| 1-388 | CCl | N | CH | CBr | H | CH₂CH₂ | OCH₂Ph |
| 1-389 | CCl | N | CH | CBr | H | CH₂CH₂ | NHSO₂Me |
| 1-390 | CCl | N | CH | CBr | H | CH₂CH₂ | NHSO₂N(Me)(CHMe₂) |
| 1-391 | CCl | N | CH | CMe | H | CH₂CH₂ | OH |
| 1-392 | CCl | N | CH | CMe | H | CH₂CH₂ | OMe |
| 1-393 | CCl | N | CH | CMe | H | CH₂CH₂ | OEt |
| 1-394 | CCl | N | CH | CMe | H | CH₂CH₂ | OCH₂Ph |
| 1-395 | CCl | N | CH | CMe | H | CH₂CH₂ | NHSO₂Me |
| 1-396 | CCl | N | CH | CMe | H | CH₂CH₂ | NHSO₂N(Me)(CHMe₂) |
| 1-397 | CCl | N | CH | CCF₂H | H | CH₂CH₂ | OH |
| 1-398 | CCl | N | CH | CCF₂H | H | CH₂CH₂ | OMe |
| 1-399 | CCl | N | CH | CCF₂H | H | CH₂CH₂ | OEt |
| 1-400 | CCl | N | CH | CCF₂H | H | CH₂CH₂ | OCH₂Ph |
| 1-401 | CCl | N | CH | CCF₂H | H | CH₂CH₂ | NHSO₂Me |
| 1-402 | CCl | N | CH | CCF₂H | H | CH₂CH₂ | NHSO₂N(Me)(CHMe₂) |
| 1-403 | CCl | N | CH | CCF₂Me | H | CH₂CH₂ | OH |
| 1-404 | CCl | N | CH | CCF₂Me | H | CH₂CH₂ | OMe |
| 1-405 | CCl | N | CH | CCF₂Me | H | CH₂CH₂ | OEt |
| 1-406 | CCl | N | CH | CCF₂Me | H | CH₂CH₂ | OCH₂Ph |
| 1-407 | CCl | N | CH | CCF₂Me | H | CH₂CH₂ | NHSO₂Me |
| 1-408 | CCl | N | CH | CCF₂Me | H | CH₂CH₂ | NHSO₂N(Me)(CHMe₂) |
| 1-409 | CCl | N | CH | CCF₃ | H | CH₂CH₂ | OH |
| 1-410 | CCl | N | CH | CCF₃ | H | CH₂CH₂ | OMe |
| 1-411 | CCl | N | CH | CCF₃ | H | CH₂CH₂ | OEt |
| 1-412 | CCl | N | CH | CCF₃ | H | CH₂CH₂ | OCH₂Ph |
| 1-413 | CCl | N | CH | CCF₃ | H | CH₂CH₂ | NHSO₂Me |
| 1-414 | CCl | N | CH | CCF₃ | H | CH₂CH₂ | NHSO₂N(Me)(CHMe₂) |
| 1-415 | CH | N | CH | CCF₃ | H | CH₂CH₂ | OH |
| 1-416 | CH | N | CH | CCF₃ | H | CH₂CH₂ | OMe |
| 1-417 | CH | N | CH | CCF₃ | H | CH₂CH₂ | OEt |
| 1-418 | CH | N | CH | CCF₃ | H | CH₂CH₂ | OCH₂Ph |
| 1-419 | CH | N | CH | CCF₃ | H | CH₂CH₂ | NHSO₂Me |
| 1-420 | CH | N | CH | CCF₃ | H | CH₂CH₂ | NHSO₂N(Me)(CHMe₂) |
| 1-421 | CF | N | CH | CCF₃ | H | CH₂CH₂ | OH |
| 1-422 | CF | N | CH | CCF₃ | H | CH₂CH₂ | OMe |
| 1-423 | CF | N | CH | CCF₃ | H | CH₂CH₂ | OEt |
| 1-424 | CF | N | CH | CCF₃ | H | CH₂CH₂ | OCH₂Ph |
| 1-425 | CF | N | CH | CCF₃ | H | CH₂CH₂ | NHSO₂Me |
| 1-426 | CF | N | CH | CCF₃ | H | CH₂CH₂ | NHSO₂N(Me)(CHMe₂) |
| 1-427 | CBr | N | CH | CCF₃ | H | CH₂CH₂ | OH |
| 1-428 | CBr | N | CH | CCF₃ | H | CH₂CH₂ | OMe |
| 1-429 | CBr | N | CH | CCF₃ | H | CH₂CH₂ | OEt |
| 1-430 | CBr | N | CH | CCF₃ | H | CH₂CH₂ | OCH₂Ph |
| 1-431 | CBr | N | CH | CCF₃ | H | CH₂CH₂ | NHSO₂Me |
| 1-432 | CBr | N | CH | CCF₃ | H | CH₂CH₂ | NHSO₂N(Me)(CHMe₂) |
| 1-433 | CCl | N | CH | CSO₂Me | H | CH₂CH₂ | OH |
| 1-434 | CCl | N | CH | CSO₂Me | H | CH₂CH₂ | OMe |
| 1-435 | CCl | N | CH | CSO₂Me | H | CH₂CH₂ | OEt |
| 1-436 | CCl | N | CH | CSO₂Me | H | CH₂CH₂ | OCH₂Ph |
| 1-437 | CCl | N | CH | CSO₂Me | H | CH₂CH₂ | NHSO₂Me |
| 1-438 | CCl | N | CH | CSO₂Me | H | CH₂CH₂ | NHSO₂N(Me)(CHMe₂) |
| 1-439 | CH | CH | N | COCF₂O | | CH₂CH₂ | OH |
| 1-440 | CH | CH | N | COCF₂O | | CH₂CH₂ | OMe |
| 1-441 | CH | CH | N | COCF₂O | | CH₂CH₂ | OEt |
| 1-442 | CH | CH | N | COCF₂O | | CH₂CH₂ | OCH₂Ph |
| 1-443 | CH | CH | N | COCF₂O | | CH₂CH₂ | NHSO₂Me |
| 1-444 | CH | CH | N | COCF₂O | | CH₂CH₂ | NHSO₂N(Me)(CHMe₂) |
| 1-445 | CH | F | N | CH | CF₃ | CH₂CH₂ | OH |
| 1-446 | CH | F | N | CH | CF₃ | CH₂CH₂ | OMe |
| 1-447 | CH | F | N | CH | CF₃ | CH₂CH₂ | OEt |
| 1-448 | CH | F | N | CH | CF₃ | CH₂CH₂ | OCH₂Ph |
| 1-449 | CH | F | N | CH | CF₃ | CH₂CH₂ | NHSO₂Me |
| 1-450 | CH | F | N | CH | CF₃ | CH₂CH₂ | NHSO₂N(Me)(CHMe₂) |
| 1-451 | CH | CCl | CCl | N | H | CH₂CH₂ | OH |
| 1-452 | CH | CCl | CCl | N | H | CH₂CH₂ | OMe |
| 1-453 | CH | CCl | CCl | N | H | CH₂CH₂ | OEt |
| 1-454 | CH | CCl | CCl | N | H | CH₂CH₂ | OCH₂Ph |
| 1-455 | CH | CCl | CCl | N | H | CH₂CH₂ | NHSO₂Me |
| 1-456 | CH | CCl | CCl | N | H | CH₂CH₂ | NHSO₂N(Me)(CHMe₂) |
| 1-457 | CCl | CH | CH | CMe | H | CHMeCH₂ | OH |
| 1-458 | CCl | CH | CH | CMe | H | CHMeCH₂ | OMe |
| 1-459 | CCl | CH | CH | CMe | H | CHMeCH₂ | OEt |
| 1-460 | CCl | CH | CH | CMe | H | CHMeCH₂ | OCH₂Ph |
| 1-461 | CCl | CH | CH | CMe | H | CHMeCH₂ | NHSO₂Me |
| 1-462 | CCl | CH | CH | CMe | H | CHMeCH₂ | NHSO₂N(Me)(CHMe₂) |
| 1-463 | N | CH | N | CCF₃ | H | CHMeCH₂ | OH |
| 1-464 | N | CH | N | CCF₃ | H | CHMeCH₂ | OMe |
| 1-465 | N | CH | N | CCF₃ | H | CHMeCH₂ | OEt |
| 1-466 | N | CH | N | CCF₃ | H | CHMeCH₂ | OCH₂Ph |
| 1-467 | N | CH | N | CCF₃ | H | CHMeCH₂ | NHSO₂Me |
| 1-468 | N | CH | N | CCF₃ | H | CHMeCH₂ | NHSO₂N(Me)(CHMe₂) |
| 1-469 | NO | CCl | CH | CCF₃ | H | CHMeCH₂ | OH |
| 1-470 | NO | CCl | CH | CCF₃ | H | CHMeCH₂ | OMe |
| 1-471 | NO | CCl | CH | CCF₃ | H | CHMeCH₂ | OEt |
| 1-472 | NO | CCl | CH | CCF₃ | H | CHMeCH₂ | OCH₂Ph |
| 1-473 | NO | CCl | CH | CCF₃ | H | CHMeCH₂ | NHSO₂Me |
| 1-474 | NO | CCl | CH | CCF₃ | H | CHMeCH₂ | NHSO₂N(Me)(CHMe₂) |
| 1-475 | CF | N | CH | CF | H | CHMeCH₂ | OH |
| 1-476 | CF | N | CH | CF | H | CHMeCH₂ | OMe |
| 1-477 | CF | N | CH | CF | H | CHMeCH₂ | OEt |
| 1-478 | CF | N | CH | CF | H | CHMeCH₂ | OCH₂Ph |
| 1-479 | CF | N | CH | CF | H | CHMeCH₂ | NHSO₂Me |
| 1-480 | CF | N | CH | CF | H | CHMeCH₂ | NHSO₂N(Me)(CHMe₂) |
| 1-481 | CCl | N | CH | CF | H | CHMeCH₂ | OH |
| 1-482 | CCl | N | CH | CF | H | CHMeCH₂ | OMe |
| 1-483 | CCl | N | CH | CF | H | CHMeCH₂ | OEt |
| 1-484 | CCl | N | CH | CF | H | CHMeCH₂ | OCH₂Ph |
| 1-485 | CCl | N | CH | CF | H | CHMeCH₂ | NHSO₂Me |
| 1-486 | CCl | N | CH | CF | H | CHMeCH₂ | NHSO₂N(Me)(CHMe₂) |
| 1-487 | CCl | N | CH | CCl | H | CHMeCH₂ | OH |
| 1-488 | CCl | N | CH | CCl | H | CHMeCH₂ | OMe |
| 1-489 | CCl | N | CH | CCl | H | CHMeCH₂ | OEt |
| 1-490 | CCl | N | CH | CCl | H | CHMeCH₂ | OCH₂Ph |
| 1-491 | CCl | N | CH | CCl | H | CHMeCH₂ | NHSO₂Me |
| 1-492 | CCl | N | CH | CCl | H | CHMeCH₂ | NHSO₂N(Me)(CHMe₂) |
| 1-493 | CCF₃ | N | CH | CCl | H | CHMeCH₂ | OH |
| 1-494 | CCF₃ | N | CH | CCl | H | CHMeCH₂ | OMe |
| 1-495 | CCF₃ | N | CH | CCl | H | CHMeCH₂ | OEt |
| 1-496 | CCF₃ | N | CH | CCl | H | CHMeCH₂ | OCH₂Ph |
| 1-497 | CCF₃ | N | CH | CCl | H | CHMeCH₂ | NHSO₂Me |
| 1-498 | CCF₃ | N | CH | CCl | H | CHMeCH₂ | NHSO₂N(Me)(CHMe₂) |
| 1-499 | CCl | N | CH | CBr | H | CHMeCH₂ | OH |
| 1-500 | CCl | N | CH | CBr | H | CHMeCH₂ | OMe |
| 1-501 | CCl | N | CH | CBr | H | CHMeCH₂ | OEt |
| 1-502 | CCl | N | CH | CBr | H | CHMeCH₂ | OCH₂Ph |
| 1-503 | CCl | N | CH | CBr | H | CHMeCH₂ | NHSO₂Me |
| 1-504 | CCl | N | CH | CBr | H | CHMeCH₂ | NHSO₂N(Me)(CHMe₂) |
| 1-505 | CCl | N | CH | CMe | H | CHMeCH₂ | OH |
| 1-506 | CCl | N | CH | CMe | H | CHMeCH₂ | OMe |
| 1-507 | CCl | N | CH | CMe | H | CHMeCH₂ | OEt |
| 1-508 | CCl | N | CH | CMe | H | CHMeCH₂ | OCH₂Ph |
| 1-509 | CCl | N | CH | CMe | H | CHMeCH₂ | NHSO₂Me |
| 1-510 | CCl | N | CH | CMe | H | CHMeCH₂ | NHSO₂N(Me)(CHMe₂) |
| 1-511 | CCl | N | CH | CCF₂H | H | CHMeCH₂ | OH |
| 1-512 | CCl | N | CH | CCF₂H | H | CHMeCH₂ | OMe |
| 1-513 | CCl | N | CH | CCF₂H | H | CHMeCH₂ | OEt |
| 1-514 | CCl | N | CH | CCF₂H | H | CHMeCH₂ | OCH₂Ph |
| 1-515 | CCl | N | CH | CCF₂H | H | CHMeCH₂ | NHSO₂Me |
| 1-516 | CCl | N | CH | CCF₂H | H | CHMeCH₂ | NHSO₂N(Me)(CHMe₂) |
| 1-517 | CCl | N | CH | CCF₂Me | H | CHMeCH₂ | OH |
| 1-518 | CCl | N | CH | CCF₂Me | H | CHMeCH₂ | OMe |
| 1-519 | CCl | N | CH | CCF₂Me | H | CHMeCH₂ | OEt |
| 1-520 | CCl | N | CH | CCF₂Me | H | CHMeCH₂ | OCH₂Ph |
| 1-521 | CCl | N | CH | CCF₂Me | H | CHMeCH₂ | NHSO₂Me |
| 1-522 | CCl | N | CH | CCF₂Me | H | CHMeCH₂ | NHSO₂N(Me)(CHMe₂) |
| 1-523 | CCl | N | CH | CCF₃ | H | CHMeCH₂ | OH |
| 1-524 | CCl | N | CH | CCF₃ | H | CHMeCH₂ | OMe |
| 1-525 | CCl | N | CH | CCF₃ | H | CHMeCH₂ | OEt |
| 1-526 | CCl | N | CH | CCF₃ | H | CHMeCH₂ | OCH₂Ph |
| 1-527 | CCl | N | CH | CCF₃ | H | CHMeCH₂ | NHSO₂Me |
| 1-528 | CCl | N | CH | CCF₃ | H | CHMeCH₂ | NHSO₂N(Me)(CHMe₂) |
| 1-529 | CH | N | CH | CCF₃ | H | CHMeCH₂ | OH |
| 1-530 | CH | N | CH | CCF₃ | H | CHMeCH₂ | OMe |
| 1-531 | CH | N | CH | CCF₃ | H | CHMeCH₂ | OEt |
| 1-532 | CH | N | CH | CCF₃ | H | CHMeCH₂ | OCH₂Ph |
| 1-533 | CH | N | CH | CCF₃ | H | CHMeCH₂ | NHSO₂Me |
| 1-534 | CH | N | CH | CCF₃ | H | CHMeCH₂ | NHSO₂N(Me)(CHMe₂) |
| 1-535 | CF | N | CH | CCF₃ | H | CHMeCH₂ | OH |
| 1-536 | CF | N | CH | CCF₃ | H | CHMeCH₂ | OMe |
| 1-537 | CF | N | CH | CCF₃ | H | CHMeCH₂ | OEt |
| 1-538 | CF | N | CH | CCF₃ | H | CHMeCH₂ | OCH₂Ph |
| 1-539 | CF | N | CH | CCF₃ | H | CHMeCH₂ | NHSO₂Me |
| 1-540 | CF | N | CH | CCF₃ | H | CHMeCH₂ | NHSO₂N(Me)(CHMe₂) |
| 1-541 | CBr | N | CH | CCF₃ | H | CHMeCH₂ | OH |
| 1-542 | CBr | N | CH | CCF₃ | H | CHMeCH₂ | OMe |
| 1-543 | CBr | N | CH | CCF₃ | H | CHMeCH₂ | OEt |
| 1-544 | CBr | N | CH | CCF₃ | H | CHMeCH₂ | OCH₂Ph |
| 1-545 | CBr | N | CH | CCF₃ | H | CHMeCH₂ | NHSO₂Me |
| 1-546 | CBr | N | CH | CCF₃ | H | CHMeCH₂ | NHSO₂N(Me)(CHMe₂) |
| 1-547 | CCl | N | CH | CSO₂Me | H | CHMeCH₂ | OH |
| 1-548 | CCl | N | CH | CSO₂Me | H | CHMeCH₂ | OMe |
| 1-549 | CCl | N | CH | CSO₂Me | H | CHMeCH₂ | OEt |
| 1-550 | CCl | N | CH | CSO₂Me | H | CHMeCH₂ | OCH₂Ph |
| 1-551 | CCl | N | CH | CSO₂Me | H | CHMeCH₂ | NHSO₂Me |
| 1-552 | CCl | N | CH | CSO₂Me | H | CHMeCH₂ | NHSO₂N(Me)(CHMe₂) |
| 1-553 | CH | CH | N | COCF₂O | | CHMeCH₂ | OH |
| 1-554 | CH | CH | N | COCF₂O | | CHMeCH₂ | OMe |
| 1-555 | CH | CH | N | COCF₂O | | CHMeCH₂ | OEt |
| 1-556 | CH | CH | N | COCF₂O | | CHMeCH₂ | OCH₂Ph |
| 1-557 | CH | CH | N | COCF₂O | | CHMeCH₂ | NHSO₂Me |
| 1-558 | CH | CH | N | COCF₂O | | CHMeCH₂ | NHSO₂N(Me)(CHMe₂) |
| 1-559 | CH | F | N | CH | CF₃ | CHMeCH₂ | OH |
| 1-560 | CH | F | N | CH | CF₃ | CHMeCH₂ | OMe |
| 1-561 | CH | F | N | CH | CF₃ | CHMeCH₂ | OEt |
| 1-562 | CH | F | N | CH | CF₃ | CHMeCH₂ | OCH₂Ph |
| 1-563 | CH | F | N | CH | CF₃ | CHMeCH₂ | NHSO₂Me |
| 1-564 | CH | F | N | CH | CF₃ | CHMeCH₂ | NHSO₂N(Me)(CHMe₂) |
| 1-565 | CH | CCl | CCl | N | H | CHMeCH₂ | OH |
| 1-566 | CH | CCl | CCl | N | H | CHMeCH₂ | OMe |
| 1-567 | CH | CCl | CCl | N | H | CHMeCH₂ | OEt |
| 1-568 | CH | CCl | CCl | N | H | CHMeCH₂ | OCH₂Ph |
| 1-569 | CH | CCl | CCl | N | H | CHMeCH₂ | NHSO₂Me |
| 1-570 | CH | CCl | CCl | N | H | CHMeCH₂ | NHSO₂N(Me)(CHMe₂) |
| 1-571 | CCl | CH | CH | CMe | H | CH₂CHMe | OH |
| 1-572 | CCl | CH | CH | CMe | H | CH₂CHMe | OMe |
| 1-573 | CCl | CH | CH | CMe | H | CH₂CHMe | OEt |
| 1-574 | CCl | CH | CH | CMe | H | CH₂CHMe | OCH₂Ph |
| 1-575 | CCl | CH | CH | CMe | H | CH₂CHMe | NHSO₂Me |
| 1-576 | CCl | CH | CH | CMe | H | CH₂CHMe | NHSO₂N(Me)(CHMe₂) |
| 1-577 | N | CH | N | CCF₃ | H | CH₂CHMe | OH |
| 1-578 | N | CH | N | CCF₃ | H | CH₂CHMe | OMe |
| 1-579 | N | CH | N | CCF₃ | H | CH₂CHMe | OEt |
| 1-580 | N | CH | N | CCF₃ | H | CH₂CHMe | OCH₂Ph |
| 1-581 | N | CH | N | CCF₃ | H | CH₂CHMe | NHSO₂Me |
| 1-582 | N | CH | N | CCF₃ | H | CH₂CHMe | NHSO₂N(Me)(CHMe₂) |
| 1-583 | NO | CCl | CH | CCF₃ | H | CH₂CHMe | OH |
| 1-584 | NO | CCl | CH | CCF₃ | H | CH₂CHMe | OMe |
| 1-585 | NO | CCl | CH | CCF₃ | H | CH₂CHMe | OEt |
| 1-586 | NO | CCl | CH | CCF₃ | H | CH₂CHMe | OCH₂Ph |
| 1-587 | NO | CCl | CH | CCF₃ | H | CH₂CHMe | NHSO₂Me |
| 1-588 | NO | CCl | CH | CCF₃ | H | CH₂CHMe | NHSO₂N(Me)(CHMe₂) |
| 1-589 | CF | N | CH | CF | H | CH₂CHMe | OH |
| 1-590 | CF | N | CH | CF | H | CH₂CHMe | OMe |
| 1-591 | CF | N | CH | CF | H | CH₂CHMe | OEt |
| 1-592 | CF | N | CH | CF | H | CH₂CHMe | OCH₂Ph |
| 1-593 | CF | N | CH | CF | H | CH₂CHMe | NHSO₂Me |
| 1-594 | CF | N | CH | CF | H | CH₂CHMe | NHSO₂N(Me)(CHMe₂) |
| 1-595 | CCl | N | CH | CF | H | CH₂CHMe | OH |
| 1-596 | CCl | N | CH | CF | H | CH₂CHMe | OMe |
| 1-597 | CCl | N | CH | CF | H | CH₂CHMe | OEt |
| 1-598 | CCl | N | CH | CF | H | CH₂CHMe | OCH₂Ph |
| 1-599 | CCl | N | CH | CF | H | CH₂CHMe | NHSO₂Me |
| 1-600 | CCl | N | CH | CF | H | CH₂CHMe | NHSO₂N(Me)(CHMe₂) |
| 1-601 | CCl | N | CH | CCl | H | CH₂CHMe | OH |
| 1-602 | CCl | N | CH | CCl | H | CH₂CHMe | OMe |
| 1-603 | CCl | N | CH | CCl | H | CH₂CHMe | OEt |
| 1-604 | CCl | N | CH | CCl | H | CH₂CHMe | OCH₂Ph |
| 1-605 | CCl | N | CH | CCl | H | CH₂CHMe | NHSO₂Me |
| 1-606 | CCl | N | CH | CCl | H | CH₂CHMe | NHSO₂N(Me)(CHMe₂) |
| 1-607 | CCF₃ | N | CH | CCl | H | CH₂CHMe | OH |
| 1-608 | CCF₃ | N | CH | CCl | H | CH₂CHMe | OMe |
| 1-609 | CCF₃ | N | CH | CCl | H | CH₂CHMe | OEt |
| 1-610 | CCF₃ | N | CH | CCl | H | CH₂CHMe | OCH₂Ph |
| 1-611 | CCF₃ | N | CH | CCl | H | CH₂CHMe | NHSO₂Me |
| 1-612 | CCF₃ | N | CH | CCl | H | CH₂CHMe | NHSO₂N(Me)(CHMe₂) |
| 1-613 | CCl | N | CH | CBr | H | CH₂CHMe | OH |
| 1-614 | CCl | N | CH | CBr | H | CH₂CHMe | OMe |
| 1-615 | CCl | N | CH | CBr | H | CH₂CHMe | OEt |
| 1-616 | CCl | N | CH | CBr | H | CH₂CHMe | OCH₂Ph |
| 1-617 | CCl | N | CH | CBr | H | CH₂CHMe | NHSO₂Me |
| 1-618 | CCl | N | CH | CBr | H | CH₂CHMe | NHSO₂N(Me)(CHMe₂) |
| 1-619 | CCl | N | CH | CMe | H | CH₂CHMe | OH |
| 1-620 | CCl | N | CH | CMe | H | CH₂CHMe | OMe |
| 1-621 | CCl | N | CH | CMe | H | CH₂CHMe | OEt |
| 1-622 | CCl | N | CH | CMe | H | CH₂CHMe | OCH₂Ph |
| 1-623 | CCl | N | CH | CMe | H | CH₂CHMe | NHSO₂Me |
| 1-624 | CCl | N | CH | CMe | H | CH₂CHMe | NHSO₂N(Me)(CHMe₂) |
| 1-625 | CCl | N | CH | CCF₂H | H | CH₂CHMe | OH |
| 1-626 | CCl | N | CH | CCF₂H | H | CH₂CHMe | OMe |
| 1-627 | CCl | N | CH | CCF₂H | H | CH₂CHMe | OEt |
| 1-628 | CCl | N | CH | CCF₂H | H | CH₂CHMe | OCH₂Ph |
| 1-629 | CCl | N | CH | CCF₂H | H | CH₂CHMe | NHSO₂Me |
| 1-630 | CCl | N | CH | CCF₂H | H | CH₂CHMe | NHSO₂N(Me)(CHMe₂) |
| 1-631 | CCl | N | CH | CCF₂Me | H | CH₂CHMe | OH |
| 1-632 | CCl | N | CH | CCF₂Me | H | CH₂CHMe | OMe |
| 1-633 | CCl | N | CH | CCF₂Me | H | CH₂CHMe | OEt |
| 1-634 | CCl | N | CH | CCF₂Me | H | CH₂CHMe | OCH₂Ph |
| 1-635 | CCl | N | CH | CCF₂Me | H | CH₂CHMe | NHSO₂Me |
| 1-636 | CCl | N | CH | CCF₂Me | H | CH₂CHMe | NHSO₂N(Me)(CHMe₂) |
| 1-637 | CCl | N | CH | CCF₃ | H | CH₂CHMe | OH |
| 1-638 | CCl | N | CH | CCF₃ | H | CH₂CHMe | OMe |
| 1-639 | CCl | N | CH | CCF₃ | H | CH₂CHMe | OEt |
| 1-640 | CCl | N | CH | CCF₃ | H | CH₂CHMe | OCH₂Ph |
| 1-641 | CCl | N | CH | CCF₃ | H | CH₂CHMe | NHSO₂Me |
| 1-642 | CCl | N | CH | CCF₃ | H | CH₂CHMe | NHSO₂N(Me)(CHMe₂) |
| 1-643 | CH | N | CH | CCF₃ | H | CH₂CHMe | OH |
| 1-644 | CH | N | CH | CCF₃ | H | CH₂CHMe | OMe |
| 1-645 | CH | N | CH | CCF₃ | H | CH₂CHMe | OEt |
| 1-646 | CH | N | CH | CCF₃ | H | CH₂CHMe | OCH₂Ph |
| 1-647 | CH | N | CH | CCF₃ | H | CH₂CHMe | NHSO₂Me |
| 1-648 | CH | N | CH | CCF₃ | H | CH₂CHMe | NHSO₂N(Me)(CHMe₂) |
| 1-649 | CF | N | CH | CCF₃ | H | CH₂CHMe | OH |
| 1-650 | CF | N | CH | CCF₃ | H | CH₂CHMe | OMe |
| 1-651 | CF | N | CH | CCF₃ | H | CH₂CHMe | OEt |
| 1-652 | CF | N | CH | CCF₃ | H | CH₂CHMe | OCH₂Ph |
| 1-653 | CF | N | CH | CCF₃ | H | CH₂CHMe | NHSO₂Me |
| 1-654 | CF | N | CH | CCF₃ | H | CH₂CHMe | NHSO₂N(Me)(CHMe₂) |
| 1-655 | CBr | N | CH | CCF₃ | H | CH₂CHMe | OH |
| 1-656 | CBr | N | CH | CCF₃ | H | CH₂CHMe | OMe |
| 1-657 | CBr | N | CH | CCF₃ | H | CH₂CHMe | OEt |
| 1-658 | CBr | N | CH | CCF₃ | H | CH₂CHMe | OCH₂Ph |
| 1-659 | CBr | N | CH | CCF₃ | H | CH₂CHMe | NHSO₂Me |
| 1-660 | CBr | N | CH | CCF₃ | H | CH₂CHMe | NHSO₂N(Me)(CHMe₂) |
| 1-661 | CCl | N | CH | CSO₂Me | H | CH₂CHMe | OH |
| 1-662 | CCl | N | CH | CSO₂Me | H | CH₂CHMe | OMe |
| 1-663 | CCl | N | CH | CSO₂Me | H | CH₂CHMe | OEt |
| 1-664 | CCl | N | CH | CSO₂Me | H | CH₂CHMe | OCH₂Ph |
| 1-665 | CCl | N | CH | CSO₂Me | H | CH₂CHMe | NHSO₂Me |
| 1-666 | CCl | N | CH | CSO₂Me | H | CH₂CHMe | NHSO₂N(Me)(CHMe₂) |
| 1-667 | CH | CH | N | COCF₂O | | CH₂CHMe | OH |
| 1-668 | CH | CH | N | COCF₂O | | CH₂CHMe | OMe |
| 1-669 | CH | CH | N | COCF₂O | | CH₂CHMe | OEt |
| 1-670 | CH | CH | N | COCF₂O | | CH₂CHMe | OCH₂Ph |
| 1-671 | CH | CH | N | COCF₂O | | CH₂CHMe | NHSO₂Me |
| 1-672 | CH | CH | N | COCF₂O | | CH₂CHMe | NHSO₂N(Me)(CHMe₂) |
| 1-673 | CH | F | N | CH | CF₃ | CH₂CHMe | OH |
| 1-674 | CH | F | N | CH | CF₃ | CH₂CHMe | OMe |
| 1-675 | CH | F | N | CH | CF₃ | CH₂CHMe | OEt |
| 1-676 | CH | F | N | CH | CF₃ | CH₂CHMe | OCH₂Ph |
| 1-677 | CH | F | N | CH | CF₃ | CH₂CHMe | NHSO₂Me |
| 1-678 | CH | F | N | CH | CF₃ | CH₂CHMe | NHSO₂N(Me)(CHMe₂) |
| 1-679 | CH | CCl | CCl | N | H | CH₂CHMe | OH |
| 1-680 | CH | CCl | CCl | N | H | CH₂CHMe | OMe |
| 1-681 | CH | CCl | CCl | N | H | CH₂CHMe | OEt |
| 1-682 | CH | CCl | CCl | N | H | CH₂CHMe | OCH₂Ph |
| 1-683 | CH | CCl | CCl | N | H | CH₂CHMe | NHSO₂Me |
| 1-684 | CH | CCl | CCl | N | H | CH₂CHMe | NHSO₂N(Me)(CHMe₂) |
| 1-685 | CCl | CH | CH | CMe | H | CMe₂CH₂ | OH |
| 1-686 | CCl | CH | CH | CMe | H | CMe₂CH₂ | OMe |
| 1-687 | CCl | CH | CH | CMe | H | CMe₂CH₂ | OEt |
| 1-688 | CCl | CH | CH | CMe | H | CMe₂CH₂ | OCH₂Ph |
| 1-689 | CCl | CH | CH | CMe | H | CMe₂CH₂ | NHSO₂Me |
| 1-690 | CCl | CH | CH | CMe | H | CMe₂CH₂ | NHSO₂N(Me)(CHMe₂) |
| 1-691 | N | CH | N | CCF₃ | H | CMe₂CH₂ | OH |
| 1-692 | N | CH | N | CCF₃ | H | CMe₂CH₂ | OMe |
| 1-693 | N | CH | N | CCF₃ | H | CMe₂CH₂ | OEt |
| 1-694 | N | CH | N | CCF₃ | H | CMe₂CH₂ | OCH₂Ph |
| 1-695 | N | CH | N | CCF₃ | H | CMe₂CH₂ | NHSO₂Me |
| 1-696 | N | CH | N | CCF₃ | H | CMe₂CH₂ | NHSO₂N(Me)(CHMe₂) |
| 1-697 | NO | CCl | CH | CCF₃ | H | CMe₂CH₂ | OH |
| 1-698 | NO | CCl | CH | CCF₃ | H | CMe₂CH₂ | OMe |
| 1-699 | NO | CCl | CH | CCF₃ | H | CMe₂CH₂ | OEt |
| 1-700 | NO | CCl | CH | CCF₃ | H | CMe₂CH₂ | OCH₂Ph |
| 1-701 | NO | CCl | CH | CCF₃ | H | CMe₂CH₂ | NHSO₂Me |
| 1-702 | NO | CCl | CH | CCF₃ | H | CMe₂CH₂ | NHSO₂N(Me)(CHMe₂) |
| 1-703 | CF | N | CH | CF | H | CMe₂CH₂ | OH |
| 1-704 | CF | N | CH | CF | H | CMe₂CH₂ | OMe |
| 1-705 | CF | N | CH | CF | H | CMe₂CH₂ | OEt |
| 1-706 | CF | N | CH | CF | H | CMe₂CH₂ | OCH₂Ph |
| 1-707 | CF | N | CH | CF | H | CMe₂CH₂ | NHSO₂Me |
| 1-708 | CF | N | CH | CF | H | CMe₂CH₂ | NHSO₂N(Me)(CHMe₂) |
| 1-709 | CCl | N | CH | CF | H | CMe₂CH₂ | OH |
| 1-710 | CCl | N | CH | CF | H | CMe₂CH₂ | OMe |
| 1-711 | CCl | N | CH | CF | H | CMe₂CH₂ | OEt |
| 1-712 | CCl | N | CH | CF | H | CMe₂CH₂ | OCH₂Ph |
| 1-713 | CCl | N | CH | CF | H | CMe₂CH₂ | NHSO₂Me |
| 1-714 | CCl | N | CH | CF | H | CMe₂CH₂ | NHSO₂N(Me)(CHMe₂) |
| 1-715 | CCl | N | CH | CCl | H | CMe₂CH₂ | OH |
| 1-716 | CCl | N | CH | CCl | H | CMe₂CH₂ | OMe |
| 1-717 | CCl | N | CH | CCl | H | CMe₂CH₂ | OEt |
| 1-718 | CCl | N | CH | CCl | H | CMe₂CH₂ | OCH₂Ph |
| 1-719 | CCl | N | CH | CCl | H | CMe₂CH₂ | NHSO₂Me |
| 1-720 | CCl | N | CH | CCl | H | CMe₂CH₂ | NHSO₂N(Me)(CHMe₂) |
| 1-721 | CCF₃ | N | CH | CCl | H | CMe₂CH₂ | OH |
| 1-722 | CCF₃ | N | CH | CCl | H | CMe₂CH₂ | OMe |
| 1-723 | CCF₃ | N | CH | CCl | H | CMe₂CH₂ | OEt |
| 1-724 | CCF₃ | N | CH | CCl | H | CMe₂CH₂ | OCH₂Ph |
| 1-725 | CCF₃ | N | CH | CCl | H | CMe₂CH₂ | NHSO₂Me |
| 1-726 | CCF₃ | N | CH | CCl | H | CMe₂CH₂ | NHSO₂N(Me)(CHMe₂) |
| 1-727 | CCl | N | CH | CBr | H | CMe₂CH₂ | OH |
| 1-728 | CCl | N | CH | CBr | H | CMe₂CH₂ | OMe |
| 1-729 | CCl | N | CH | CBr | H | CMe₂CH₂ | OEt |
| 1-730 | CCl | N | CH | CBr | H | CMe₂CH₂ | OCH₂Ph |
| 1-731 | CCl | N | CH | CBr | H | CMe₂CH₂ | NHSO₂Me |
| 1-732 | CCl | N | CH | CBr | H | CMe₂CH₂ | NHSO₂N(Me)(CHMe₂) |
| 1-733 | CCl | N | CH | CMe | H | CMe₂CH₂ | OH |
| 1-734 | CCl | N | CH | CMe | H | CMe₂CH₂ | OMe |
| 1-735 | CCl | N | CH | CMe | H | CMe₂CH₂ | OEt |
| 1-736 | CCl | N | CH | CMe | H | CMe₂CH₂ | OCH₂Ph |
| 1-737 | CCl | N | CH | CMe | H | CMe₂CH₂ | NHSO₂Me |
| 1-738 | CCl | N | CH | CMe | H | CMe₂CH₂ | NHSO₂N(Me)(CHMe₂) |
| 1-739 | CCl | N | CH | CCF₂H | H | CMe₂CH₂ | OH |
| 1-740 | CCl | N | CH | CCF₂H | H | CMe₂CH₂ | OMe |
| 1-741 | CCl | N | CH | CCF₂H | H | CMe₂CH₂ | OEt |
| 1-742 | CCl | N | CH | CCF₂H | H | CMe₂CH₂ | OCH₂Ph |
| 1-743 | CCl | N | CH | CCF₂H | H | CMe₂CH₂ | NHSO₂Me |
| 1-744 | CCl | N | CH | CCF₂H | H | CMe₂CH₂ | NHSO₂N(Me)(CHMe₂) |
| 1-745 | CCl | N | CH | CCF₂Me | H | CMe₂CH₂ | OH |
| 1-746 | CCl | N | CH | CCF₂Me | H | CMe₂CH₂ | OMe |
| 1-747 | CCl | N | CH | CCF₂Me | H | CMe₂CH₂ | OEt |
| 1-748 | CCl | N | CH | CCF₂Me | H | CMe₂CH₂ | OCH₂Ph |
| 1-749 | CCl | N | CH | CCF₂Me | H | CMe₂CH₂ | NHSO₂Me |
| 1-750 | CCl | N | CH | CCF₂Me | H | CMe₂CH₂ | NHSO₂N(Me)(CHMe₂) |
| 1-751 | CCl | N | CH | CCF₃ | H | CMe₂CH₂ | OH |
| 1-752 | CCl | N | CH | CCF₃ | H | CMe₂CH₂ | OMe |
| 1-753 | CCl | N | CH | CCF₃ | H | CMe₂CH₂ | OEt |
| 1-754 | CCl | N | CH | CCF₃ | H | CMe₂CH₂ | OCH₂Ph |
| 1-755 | CCl | N | CH | CCF₃ | H | CMe₂CH₂ | NHSO₂Me |
| 1-756 | CCl | N | CH | CCF₃ | H | CMe₂CH₂ | NHSO₂N(Me)(CHMe₂) |
| 1-757 | CH | N | CH | CCF₃ | H | CMe₂CH₂ | OH |
| 1-758 | CH | N | CH | CCF₃ | H | CMe₂CH₂ | OMe |
| 1-759 | CH | N | CH | CCF₃ | H | CMe₂CH₂ | OEt |
| 1-760 | CH | N | CH | CCF₃ | H | CMe₂CH₂ | OCH₂Ph |
| 1-761 | CH | N | CH | CCF₃ | H | CMe₂CH₂ | NHSO₂Me |
| 1-762 | CH | N | CH | CCF₃ | H | CMe₂CH₂ | NHSO₂N(Me)(CHMe₂) |
| 1-763 | CF | N | CH | CCF₃ | H | CMe₂CH₂ | OH |
| 1-764 | CF | N | CH | CCF₃ | H | CMe₂CH₂ | OMe |
| 1-765 | CF | N | CH | CCF₃ | H | CMe₂CH₂ | OEt |
| 1-766 | CF | N | CH | CCF₃ | H | CMe₂CH₂ | OCH₂Ph |
| 1-767 | CF | N | CH | CCF₃ | H | CMe₂CH₂ | NHSO₂Me |
| 1-768 | CF | N | CH | CCF₃ | H | CMe₂CH₂ | NHSO₂N(Me)(CHMe₂) |
| 1-769 | CBr | N | CH | CCF₃ | H | CMe₂CH₂ | OH |
| 1-770 | CBr | N | CH | CCF₃ | H | CMe₂CH₂ | OMe |
| 1-771 | CBr | N | CH | CCF₃ | H | CMe₂CH₂ | OEt |
| 1-772 | CBr | N | CH | CCF₃ | H | CMe₂CH₂ | OCH₂Ph |
| 1-773 | CBr | N | CH | CCF₃ | H | CMe₂CH₂ | NHSO₂Me |
| 1-774 | CBr | N | CH | CCF₃ | H | CMe₂CH₂ | NHSO₂N(Me)(CHMe₂) |
| 1-775 | CCl | N | CH | CSO₂Me | H | CMe₂CH₂ | OH |
| 1-776 | CCl | N | CH | CSO₂Me | H | CMe₂CH₂ | OMe |
| 1-777 | CCl | N | CH | CSO₂Me | H | CMe₂CH₂ | OEt |
| 1-778 | CCl | N | CH | CSO₂Me | H | CMe₂CH₂ | OCH₂Ph |
| 1-779 | CCl | N | CH | CSO₂Me | H | CMe₂CH₂ | NHSO₂Me |
| 1-780 | CCl | N | CH | CSO₂Me | H | CMe₂CH₂ | NHSO₂N(Me)(CHMe₂) |
| 1-781 | CH | CH | N | COCF₂O | | CMe₂CH₂ | OH |
| 1-782 | CH | CH | N | COCF₂O | | CMe₂CH₂ | OMe |
| 1-783 | CH | CH | N | COCF₂O | | CMe₂CH₂ | OEt |
| 1-784 | CH | CH | N | COCF₂O | | CMe₂CH₂ | OCH₂Ph |
| 1-785 | CH | CH | N | COCF₂O | | CMe₂CH₂ | NHSO₂Me |
| 1-786 | CH | CH | N | COCF₂O | | CMe₂CH₂ | NHSO₂N(Me)(CHMe₂) |
| 1-787 | CH | F | N | CH | CF₃ | CMe₂CH₂ | OH |
| 1-788 | CH | F | N | CH | CF₃ | CMe₂CH₂ | OMe |
| 1-789 | CH | F | N | CH | CF₃ | CMe₂CH₂ | OEt |
| 1-790 | CH | F | N | CH | CF₃ | CMe₂CH₂ | OCH₂Ph |
| 1-791 | CH | F | N | CH | CF₃ | CMe₂CH₂ | NHSO₂Me |
| 1-792 | CH | F | N | CH | CF₃ | CMe₂CH₂ | NHSO₂N(Me)(CHMe₂) |
| 1-793 | CH | CCl | CCl | N | H | CMe₂CH₂ | OH |
| 1-794 | CH | CCl | CCl | N | H | CMe₂CH₂ | OMe |
| 1-795 | CH | CCl | CCl | N | H | CMe₂CH₂ | OEt |
| 1-796 | CH | CCl | CCl | N | H | CMe₂CH₂ | OCH₂Ph |
| 1-797 | CH | CCl | CCl | N | H | CMe₂CH₂ | NHSO₂Me |
| 1-798 | CH | CCl | CCl | N | H | CMe₂CH₂ | NHSO₂N(Me)(CHMe₂) |
| 1-799 | CCl | CH | CH | CMe | H | CH₂CMe₂ | OH |
| 1-800 | CCl | CH | CH | CMe | H | CH₂CMe₂ | OMe |
| 1-801 | CCl | CH | CH | CMe | H | CH₂CMe₂ | OEt |
| 1-802 | CCl | CH | CH | CMe | H | CH₂CMe₂ | OCH₂Ph |
| 1-803 | CCl | CH | CH | CMe | H | CH₂CMe₂ | NHSO₂Me |
| 1-804 | CCl | CH | CH | CMe | H | CH₂CMe₂ | NHSO₂N(Me)(CHMe₂) |
| 1-805 | N | CH | N | CCF₃ | H | CH₂CMe₂ | OH |
| 1-806 | N | CH | N | CCF₃ | H | CH₂CMe₂ | OMe |
| 1-807 | N | CH | N | CCF₃ | H | CH₂CMe₂ | OEt |
| 1-808 | N | CH | N | CCF₃ | H | CH₂CMe₂ | OCH₂Ph |
| 1-809 | N | CH | N | CCF₃ | H | CH₂CMe₂ | NHSO₂Me |
| 1-810 | N | CH | N | CCF₃ | H | CH₂CMe₂ | NHSO₂N(Me)(CHMe₂) |
| 1-811 | NO | CCl | CH | CCF₃ | H | CH₂CMe₂ | OH |
| 1-812 | NO | CCl | CH | CCF₃ | H | CH₂CMe₂ | OMe |
| 1-813 | NO | CCl | CH | CCF₃ | H | CH₂CMe₂ | OEt |
| 1-814 | NO | CCl | CH | CCF₃ | H | CH₂CMe₂ | OCH₂Ph |
| 1-815 | NO | CCl | CH | CCF₃ | H | CH₂CMe₂ | NHSO₂Me |
| 1-816 | NO | CCl | CH | CCF₃ | H | CH₂CMe₂ | NHSO₂N(Me)(CHMe₂) |
| 1-817 | CF | N | CH | CF | H | CH₂CMe₂ | OH |
| 1-818 | CF | N | CH | CF | H | CH₂CMe₂ | OMe |
| 1-819 | CF | N | CH | CF | H | CH₂CMe₂ | OEt |
| 1-820 | CF | N | CH | CF | H | CH₂CMe₂ | OCH₂Ph |
| 1-821 | CF | N | CH | CF | H | CH₂CMe₂ | NHSO₂Me |
| 1-822 | CF | N | CH | CF | H | CH₂CMe₂ | NHSO₂N(Me)(CHMe₂) |
| 1-823 | CCl | N | CH | CF | H | CH₂CMe₂ | OH |
| 1-824 | CCl | N | CH | CF | H | CH₂CMe₂ | OMe |
| 1-825 | CCl | N | CH | CF | H | CH₂CMe₂ | OEt |
| 1-826 | CCl | N | CH | CF | H | CH₂CMe₂ | OCH₂Ph |
| 1-827 | CCl | N | CH | CF | H | CH₂CMe₂ | NHSO₂Me |
| 1-828 | CCl | N | CH | CF | H | CH₂CMe₂ | NHSO₂N(Me)(CHMe₂) |
| 1-829 | CCl | N | CH | CCl | H | CH₂CMe₂ | OH |
| 1-830 | CCl | N | CH | CCl | H | CH₂CMe₂ | OMe |
| 1-831 | CCl | N | CH | CCl | H | CH₂CMe₂ | OEt |
| 1-832 | CCl | N | CH | CCl | H | CH₂CMe₂ | OCH₂Ph |
| 1-833 | CCl | N | CH | CCl | H | CH₂CMe₂ | NHSO₂Me |
| 1-834 | CCl | N | CH | CCl | H | CH₂CMe₂ | NHSO₂N(Me)(CHMe₂) |
| 1-835 | CCF₃ | N | CH | CCl | H | CH₂CMe₂ | OH |
| 1-836 | CCF₃ | N | CH | CCl | H | CH₂CMe₂ | OMe |
| 1-837 | CCF₃ | N | CH | CCl | H | CH₂CMe₂ | OEt |
| 1-838 | CCF₃ | N | CH | CCl | H | CH₂CMe₂ | OCH₂Ph |
| 1-839 | CCF₃ | N | CH | CCl | H | CH₂CMe₂ | NHSO₂Me |
| 1-840 | CCF₃ | N | CH | CCl | H | CH₂CMe₂ | NHSO₂N(Me)(CHMe₂) |
| 1-841 | CCl | N | CH | CBr | H | CH₂CMe₂ | OH |
| 1-842 | CCl | N | CH | CBr | H | CH₂CMe₂ | OMe |
| 1-843 | CCl | N | CH | CBr | H | CH₂CMe₂ | OEt |
| 1-844 | CCl | N | CH | CBr | H | CH₂CMe₂ | OCH₂Ph |
| 1-845 | CCl | N | CH | CBr | H | CH₂CMe₂ | NHSO₂Me |
| 1-846 | CCl | N | CH | CBr | H | CH₂CMe₂ | NHSO₂N(Me)(CHMe₂) |
| 1-847 | CCl | N | CH | CMe | H | CH₂CMe₂ | OH |
| 1-848 | CCl | N | CH | CMe | H | CH₂CMe₂ | OMe |
| 1-849 | CCl | N | CH | CMe | H | CH₂CMe₂ | OEt |
| 1-850 | CCl | N | CH | CMe | H | CH₂CMe₂ | OCH₂Ph |
| 1-851 | CCl | N | CH | CMe | H | CH₂CMe₂ | NHSO₂Me |
| 1-852 | CCl | N | CH | CMe | H | CH₂CMe₂ | NHSO₂N(Me)(CHMe₂) |
| 1-853 | CCl | N | CH | CCF₂H | H | CH₂CMe₂ | OH |
| 1-854 | CCl | N | CH | CCF₂H | H | CH₂CMe₂ | OMe |
| 1-855 | CCl | N | CH | CCF₂H | H | CH₂CMe₂ | OEt |
| 1-856 | CCl | N | CH | CCF₂H | H | CH₂CMe₂ | OCH₂Ph |
| 1-857 | CCl | N | CH | CCF₂H | H | CH₂CMe₂ | NHSO₂Me |
| 1-858 | CCl | N | CH | CCF₂H | H | CH₂CMe₂ | NHSO₂N(Me)(CHMe₂) |
| 1-859 | CCl | N | CH | CCF₂Me | H | CH₂CMe₂ | OH |
| 1-860 | CCl | N | CH | CCF₂Me | H | CH₂CMe₂ | OMe |
| 1-861 | CCl | N | CH | CCF₂Me | H | CH₂CMe₂ | OEt |
| 1-862 | CCl | N | CH | CCF₂Me | H | CH₂CMe₂ | OCH₂Ph |
| 1-863 | CCl | N | CH | CCF₂Me | H | CH₂CMe₂ | NHSO₂Me |
| 1-864 | CCl | N | CH | CCF₂Me | H | CH₂CMe₂ | NHSO₂N(Me)(CHMe₂) |
| 1-865 | CCl | N | CH | CCF₃ | H | CH₂CMe₂ | OH |
| 1-866 | CCl | N | CH | CCF₃ | H | CH₂CMe₂ | OMe |
| 1-867 | CCl | N | CH | CCF₃ | H | CH₂CMe₂ | OEt |
| 1-868 | CCl | N | CH | CCF₃ | H | CH₂CMe₂ | OCH₂Ph |
| 1-869 | CCl | N | CH | CCF₃ | H | CH₂CMe₂ | NHSO₂Me |
| 1-870 | CCl | N | CH | CCF₃ | H | CH₂CMe₂ | NHSO₂N(Me)(CHMe₂) |
| 1-871 | CH | N | CH | CCF₃ | H | CH₂CMe₂ | OH |
| 1-872 | CH | N | CH | CCF₃ | H | CH₂CMe₂ | OMe |
| 1-873 | CH | N | CH | CCF₃ | H | CH₂CMe₂ | OEt |
| 1-874 | CH | N | CH | CCF₃ | H | CH₂CMe₂ | OCH₂Ph |
| 1-875 | CH | N | CH | CCF₃ | H | CH₂CMe₂ | NHSO₂Me |
| 1-876 | CH | N | CH | CCF₃ | H | CH₂CMe₂ | NHSO₂N(Me)(CHMe₂) |
| 1-877 | CF | N | CH | CCF₃ | H | CH₂CMe₂ | OH |
| 1-878 | CF | N | CH | CCF₃ | H | CH₂CMe₂ | OMe |
| 1-879 | CF | N | CH | CCF₃ | H | CH₂CMe₂ | OEt |
| 1-880 | CF | N | CH | CCF₃ | H | CH₂CMe₂ | OCH₂Ph |
| 1-881 | CF | N | CH | CCF₃ | H | CH₂CMe₂ | NHSO₂Me |
| 1-882 | CF | N | CH | CCF₃ | H | CH₂CMe₂ | NHSO₂N(Me)(CHMe₂) |
| 1-883 | CBr | N | CH | CCF₃ | H | CH₂CMe₂ | OH |
| 1-884 | CBr | N | CH | CCF₃ | H | CH₂CMe₂ | OMe |
| 1-885 | CBr | N | CH | CCF₃ | H | CH₂CMe₂ | OEt |
| 1-886 | CBr | N | CH | CCF₃ | H | CH₂CMe₂ | OCH₂Ph |
| 1-887 | CBr | N | CH | CCF₃ | H | CH₂CMe₂ | NHSO₂Me |
| 1-888 | CBr | N | CH | CCF₃ | H | CH₂CMe₂ | NHSO₂N(Me)(CHMe₂) |
| 1-889 | CCl | N | CH | CSO₂Me | H | CH₂CMe₂ | OH |
| 1-890 | CCl | N | CH | CSO₂Me | H | CH₂CMe₂ | OMe |
| 1-891 | CCl | N | CH | CSO₂Me | H | CH₂CMe₂ | OEt |
| 1-892 | CCl | N | CH | CSO₂Me | H | CH₂CMe₂ | OCH₂Ph |
| 1-893 | CCl | N | CH | CSO₂Me | H | CH₂CMe₂ | NHSO₂Me |
| 1-894 | CCl | N | CH | CSO₂Me | H | CH₂CMe₂ | NHSO₂N(Me)(CHMe₂) |
| 1-895 | CH | CH | N | COCF₂O | | CH₂CMe₂ | OH |
| 1-896 | CH | CH | N | COCF₂O | | CH₂CMe₂ | OMe |
| 1-897 | CH | CH | N | COCF₂O | | CH₂CMe₂ | OEt |
| 1-898 | CH | CH | N | COCF₂O | | CH₂CMe₂ | OCH₂Ph |
| 1-899 | CH | CH | N | COCF₂O | | CH₂CMe₂ | NHSO₂Me |
| 1-900 | CH | CH | N | COCF₂O | | CH₂CMe₂ | NHSO₂N(Me)(CHMe₂) |
| 1-901 | CH | F | N | CH | CF₃ | CH₂CMe₂ | OH |
| 1-902 | CH | F | N | CH | CF₃ | CH₂CMe₂ | OMe |
| 1-903 | CH | F | N | CH | CF₃ | CH₂CMe₂ | OEt |
| 1-904 | CH | F | N | CH | CF₃ | CH₂CMe₂ | OCH₂Ph |
| 1-905 | CH | F | N | CH | CF₃ | CH₂CMe₂ | NHSO₂Me |
| 1-906 | CH | F | N | CH | CF₃ | CH₂CMe₂ | NHSO₂N(Me)(CHMe₂) |
| 1-907 | CH | CCl | CCl | N | H | CH₂CMe₂ | OH |
| 1-908 | CH | CCl | CCl | N | H | CH₂CMe₂ | OMe |
| 1-909 | CH | CCl | CCl | N | H | CH₂CMe₂ | OEt |
| 1-910 | CH | CCl | CCl | N | H | CH₂CMe₂ | OCH₂Ph |
| 1-911 | CH | CCl | CCl | N | H | CH₂CMe₂ | NHSO₂Me |
| 1-912 | CH | CCl | CCl | N | H | CH₂CMe₂ | NHSO₂N(Me)(CHMe₂) |
| 1-913 | CCl | CH | CH | CMe | H | CH₂CMe₂ | OH |
| 1-914 | CCl | CH | CH | CMe | H | CH₂CMe₂ | OMe |
| 1-915 | CCl | CH | CH | CMe | H | CH₂CMe₂ | OEt |
| 1-916 | CCl | CH | CH | CMe | H | CH₂CMe₂ | OCH₂Ph |
| 1-917 | CCl | CH | CH | CMe | H | CH₂CMe₂ | NHSO₂Me |
| 1-918 | CCl | CH | CH | CMe | H | CH₂CMe₂ | NHSO₂N(Me)(CHMe₂) |
| 1-919 | N | CH | N | CCF₃ | H | CH₂CH₂CH₂ | OH |
| 1-920 | N | CH | N | CCF₃ | H | CH₂CH₂CH₂ | OMe |
| 1-921 | N | CH | N | CCF₃ | H | CH₂CH₂CH₂ | OEt |
| 1-922 | N | CH | N | CCF₃ | H | CH₂CH₂CH₂ | OCH₂Ph |
| 1-923 | N | CH | N | CCF₃ | H | CH₂CH₂CH₂ | NHSO₂Me |
| 1-924 | N | CH | N | CCF₃ | H | CH₂CH₂CH₂ | NHSO₂N(Me)(CHMe₂) |
| 1-925 | NO | CCl | CH | CCF₃ | H | CH₂CH₂CH₂ | OH |
| 1-926 | NO | CCl | CH | CCF₃ | H | CH₂CH₂CH₂ | OMe |
| 1-927 | NO | CCl | CH | CCF₃ | H | CH₂CH₂CH₂ | OEt |
| 1-928 | NO | CCl | CH | CCF₃ | H | CH₂CH₂CH₂ | OCH₂Ph |
| 1-929 | NO | CCl | CH | CCF₃ | H | CH₂CH₂CH₂ | NHSO₂Me |
| 1-930 | NO | CCl | CH | CCF₃ | H | CH₂CH₂CH₂ | NHSO₂N(Me)(CHMe₂) |
| 1-931 | CF | N | CH | CF | H | CH₂CH₂CH₂ | OH |
| 1-932 | CF | N | CH | CF | H | CH₂CH₂CH₂ | OMe |
| 1-933 | CF | N | CH | CF | H | CH₂CH₂CH₂ | OEt |
| 1-934 | CF | N | CH | CF | H | CH₂CH₂CH₂ | OCH₂Ph |
| 1-935 | CF | N | CH | CF | H | CH₂CH₂CH₂ | NHSO₂Me |
| 1-936 | CF | N | CH | CF | H | CH₂CH₂CH₂ | NHSO₂N(Me)(CHMe₂) |
| 1-937 | CCl | N | CH | CF | H | CH₂CH₂CH₂ | OH |
| 1-938 | CCl | N | CH | CF | H | CH₂CH₂CH₂ | OMe |
| 1-939 | CCl | N | CH | CF | H | CH₂CH₂CH₂ | OEt |
| 1-940 | CCl | N | CH | CF | H | CH₂CH₂CH₂ | OCH₂Ph |
| 1-941 | CCl | N | CH | CF | H | CH₂CH₂CH₂ | NHSO₂Me |
| 1-942 | CCl | N | CH | CF | H | CH₂CH₂CH₂ | NHSO₂N(Me)(CHMe₂) |
| 1-943 | CCl | N | CH | CCl | H | CH₂CH₂CH₂ | OH |
| 1-944 | CCl | N | CH | CCl | H | CH₂CH₂CH₂ | OMe |
| 1-945 | CCl | N | CH | CCl | H | CH₂CH₂CH₂ | OEt |
| 1-946 | CCl | N | CH | CCl | H | CH₂CH₂CH₂ | OCH₂Ph |
| 1-947 | CCl | N | CH | CCl | H | CH₂CH₂CH₂ | NHSO₂Me |
| 1-948 | CCl | N | CH | CCl | H | CH₂CH₂CH₂ | NHSO₂N(Me)(CHMe₂) |
| 1-949 | CCF₃ | N | CH | CCl | H | CH₂CH₂CH₂ | OH |
| 1-950 | CCF₃ | N | CH | CCl | H | CH₂CH₂CH₂ | OMe |
| 1-951 | CCF₃ | N | CH | CCl | H | CH₂CH₂CH₂ | OEt |
| 1-952 | CCF₃ | N | CH | CCl | H | CH₂CH₂CH₂ | OCH₂Ph |
| 1-953 | CCF₃ | N | CH | CCl | H | CH₂CH₂CH₂ | NHSO₂Me |
| 1-954 | CCF₃ | N | CH | CCl | H | CH₂CH₂CH₂ | NHSO₂N(Me)(CHMe₂) |
| 1-955 | CCl | N | CH | CBr | H | CH₂CH₂CH₂ | OH |
| 1-956 | CCl | N | CH | CBr | H | CH₂CH₂CH₂ | OMe |
| 1-957 | CCl | N | CH | CBr | H | CH₂CH₂CH₂ | OEt |
| 1-958 | CCl | N | CH | CBr | H | CH₂CH₂CH₂ | OCH₂Ph |
| 1-959 | CCl | N | CH | CBr | H | CH₂CH₂CH₂ | NHSO₂Me |
| 1-960 | CCl | N | CH | CBr | H | CH₂CH₂CH₂ | NHSO₂N(Me)(CHMe₂) |
| 1-961 | CCl | N | CH | CMe | H | CH₂CH₂CH₂ | OH |
| 1-962 | CCl | N | CH | CMe | H | CH₂CH₂CH₂ | OMe |
| 1-963 | CCl | N | CH | CMe | H | CH₂CH₂CH₂ | OEt |
| 1-964 | CCl | N | CH | CMe | H | CH₂CH₂CH₂ | OCH₂Ph |
| 1-965 | CCl | N | CH | CMe | H | CH₂CH₂CH₂ | NHSO₂Me |
| 1-966 | CCl | N | CH | CMe | H | CH₂CH₂CH₂ | NHSO₂N(Me)(CHMe₂) |
| 1-967 | CCl | N | CH | CCF₂H | H | CH₂CH₂CH₂ | OH |
| 1-968 | CCl | N | CH | CCF₂H | H | CH₂CH₂CH₂ | OMe |
| 1-969 | CCl | N | CH | CCF₂H | H | CH₂CH₂CH₂ | OEt |
| 1-970 | CCl | N | CH | CCF₂H | H | CH₂CH₂CH₂ | OCH₂Ph |
| 1-971 | CCl | N | CH | CCF₂H | H | CH₂CH₂CH₂ | NHSO₂Me |
| 1-972 | CCl | N | CH | CCF₂H | H | CH₂CH₂CH₂ | NHSO₂N(Me)(CHMe₂) |
| 1-973 | CCl | N | CH | CCF₂Me | H | CH₂CH₂CH₂ | OH |
| 1-974 | CCl | N | CH | CCF₂Me | H | CH₂CH₂CH₂ | OMe |
| 1-975 | CCl | N | CH | CCF₂Me | H | CH₂CH₂CH₂ | OEt |
| 1-976 | CCl | N | CH | CCF₂Me | H | CH₂CH₂CH₂ | OCH₂Ph |
| 1-977 | CCl | N | CH | CCF₂Me | H | CH₂CH₂CH₂ | NHSO₂Me |
| 1-978 | CCl | N | CH | CCF₂Me | H | CH₂CH₂CH₂ | NHSO₂N(Me)(CHMe₂) |
| 1-979 | CCl | N | CH | CCF₃ | H | CH₂CH₂CH₂ | OH |
| 1-980 | CCl | N | CH | CCF₃ | H | CH₂CH₂CH₂ | OMe |
| 1-981 | CCl | N | CH | CCF₃ | H | CH₂CH₂CH₂ | OEt |
| 1-982 | CCl | N | CH | CCF₃ | H | CH₂CH₂CH₂ | OCH₂Ph |
| 1-983 | CCl | N | CH | CCF₃ | H | CH₂CH₂CH₂ | NHSO₂Me |
| 1-984 | CCl | N | CH | CCF₃ | H | CH₂CH₂CH₂ | NHSO₂N(Me)(CHMe₂) |
| 1-985 | CH | N | CH | CCF₃ | H | CH₂CH₂CH₂ | OH |
| 1-986 | CH | N | CH | CCF₃ | H | CH₂CH₂CH₂ | OMe |
| 1-987 | CH | N | CH | CCF₃ | H | CH₂CH₂CH₂ | OEt |
| 1-988 | CH | N | CH | CCF₃ | H | CH₂CH₂CH₂ | OCH₂Ph |
| 1-989 | CH | N | CH | CCF₃ | H | CH₂CH₂CH₂ | NHSO₂Me |
| 1-990 | CH | N | CH | CCF₃ | H | CH₂CH₂CH₂ | NHSO₂N(Me)(CHMe₂) |
| 1-991 | CF | N | CH | CCF₃ | H | CH₂CH₂CH₂ | OH |
| 1-992 | CF | N | CH | CCF₃ | H | CH₂CH₂CH₂ | OMe |
| 1-993 | CF | N | CH | CCF₃ | H | CH₂CH₂CH₂ | OEt |
| 1-994 | CF | N | CH | CCF₃ | H | CH₂CH₂CH₂ | OCH₂Ph |
| 1-995 | CF | N | CH | CCF₃ | H | CH₂CH₂CH₂ | NHSO₂Me |
| 1-996 | CF | N | CH | CCF₃ | H | CH₂CH₂CH₂ | NHSO₂N(Me)(CHMe₂) |
| 1-997 | CBr | N | CH | CCF₃ | H | CH₂CH₂CH₂ | OH |
| 1-998 | CBr | N | CH | CCF₃ | H | CH₂CH₂CH₂ | OMe |
| 1-999 | CBr | N | CH | CCF₃ | H | CH₂CH₂CH₂ | OEt |
| 1-1000 | CBr | N | CH | CCF₃ | H | CH₂CH₂CH₂ | OCH₂Ph |
| 1-1001 | CBr | N | CH | CCF₃ | H | CH₂CH₂CH₂ | NHSO₂Me |
| 1-1002 | CBr | N | CH | CCF₃ | H | CH₂CH₂CH₂ | NHSO₂N(Me)(CHMe₂) |
| 1-1003 | CCl | N | CH | CSO₂Me | H | CH₂CH₂CH₂ | OH |
| 1-1004 | CCl | N | CH | CSO₂Me | H | CH₂CH₂CH₂ | OMe |
| 1-1005 | CCl | N | CH | CSO₂Me | H | CH₂CH₂CH₂ | OEt |
| 1-1006 | CCl | N | CH | CSO₂Me | H | CH₂CH₂CH₂ | OCH₂Ph |
| 1-1007 | CCl | N | CH | CSO₂Me | H | CH₂CH₂CH₂ | NHSO₂Me |
| 1-1008 | CCl | N | CH | CSO₂Me | H | CH₂CH₂CH₂ | NHSO₂N(Me)(CHMe₂) |
| 1-1009 | CH | CH | N | COCF₂O | | CH₂CH₂CH₂ | OH |
| 1-1010 | CH | CH | N | COCF₂O | | CH₂CH₂CH₂ | OMe |
| 1-1011 | CH | CH | N | COCF₂O | | CH₂CH₂CH₂ | OEt |
| 1-1012 | CH | CH | N | COCF₂O | | CH₂CH₂CH₂ | OCH₂Ph |
| 1-1013 | CH | CH | N | COCF₂O | | CH₂CH₂CH₂ | NHSO₂Me |
| 1-1014 | CH | CH | N | COCF₂O | | CH₂CH₂CH₂ | NHSO₂N(Me)(CHMe₂) |
| 1-1015 | CH | F | N | CH | CF₃ | CH₂CH₂CH₂ | OH |
| 1-1016 | CH | F | N | CH | CF₃ | CH₂CH₂CH₂ | OMe |
| 1-1017 | CH | F | N | CH | CF₃ | CH₂CH₂CH₂ | OEt |
| 1-1018 | CH | F | N | CH | CF₃ | CH₂CH₂CH₂ | OCH₂Ph |
| 1-1019 | CH | F | N | CH | CF₃ | CH₂CH₂CH₂ | NHSO₂Me |
| 1-1020 | CH | F | N | CH | CF₃ | CH₂CH₂CH₂ | NHSO₂N(Me)(CHMe₂) |
| 1-1021 | CH | CCl | CCl | N | H | CH₂CH₂CH₂ | OH |
| 1-1022 | CH | CCl | CCl | N | H | CH₂CH₂CH₂ | OMe |
| 1-1023 | CH | CCl | CCl | N | H | CH₂CH₂CH₂ | OEt |
| 1-1024 | CH | CCl | CCl | N | H | CH₂CH₂CH₂ | OCH₂Ph |
| 1-1025 | CH | CCl | CCl | N | H | CH₂CH₂CH₂ | NHSO₂Me |
| 1-1026 | CH | CCl | CCl | N | H | CH₂CH₂CH₂ | NHSO₂N(Me)(CHMe₂) |
| 1-1027 | CCl | CH | CH | CMe | H | CH₂CH₂CH₂CH₂ | OH |
| 1-1028 | CCl | CH | CH | CMe | H | CH₂CH₂CH₂CH₂ | OMe |
| 1-1029 | CCl | CH | CH | CMe | H | CH₂CH₂CH₂CH₂ | OEt |
| 1-1030 | CCl | CH | CH | CMe | H | CH₂CH₂CH₂CH₂ | OCH₂Ph |
| 1-1031 | CCl | CH | CH | CMe | H | CH₂CH₂CH₂CH₂ | NHSO₂Me |
| 1-1032 | CCl | CH | CH | CMe | H | CH₂CH₂CH₂CH₂ | NHSO₂N(Me)(CHMe₂) |
| 1-1033 | N | CH | N | CCF₃ | H | CH₂CH₂CH₂CH₂ | OH |
| 1-1034 | N | CH | N | CCF₃ | H | CH₂CH₂CH₂CH₂ | OMe |
| 1-1035 | N | CH | N | CCF₃ | H | CH₂CH₂CH₂CH₂ | OEt |
| 1-1036 | N | CH | N | CCF₃ | H | CH₂CH₂CH₂CH₂ | OCH₂Ph |
| 1-1037 | N | CH | N | CCF₃ | H | CH₂CH₂CH₂CH₂ | NHSO₂Me |
| 1-1038 | N | CH | N | CCF₃ | H | CH₂CH₂CH₂CH₂ | NHSO₂N(Me)(CHMe₂) |
| 1-1039 | NO | CCl | CH | CCF₃ | H | CH₂CH₂CH₂CH₂ | OH |
| 1-1040 | NO | CCl | CH | CCF₃ | H | CH₂CH₂CH₂CH₂ | OMe |
| 1-1041 | NO | CCl | CH | CCF₃ | H | CH₂CH₂CH₂CH₂ | OEt |
| 1-1042 | NO | CCl | CH | CCF₃ | H | CH₂CH₂CH₂CH₂ | OCH₂Ph |
| 1-1043 | NO | CCl | CH | CCF₃ | H | CH₂CH₂CH₂CH₂ | NHSO₂Me |
| 1-1044 | NO | CCl | CH | CCF₃ | H | CH₂CH₂CH₂CH₂ | NHSO₂N(Me)(CHMe₂) |
| 1-1045 | CF | N | CH | CF | H | CH₂CH₂CH₂CH₂ | OH |
| 1-1046 | CF | N | CH | CF | H | CH₂CH₂CH₂CH₂ | OMe |
| 1-1047 | CF | N | CH | CF | H | CH₂CH₂CH₂CH₂ | OEt |
| 1-1048 | CF | N | CH | CF | H | CH₂CH₂CH₂CH₂ | OCH₂Ph |
| 1-1049 | CF | N | CH | CF | H | CH₂CH₂CH₂CH₂ | NHSO₂Me |
| 1-1050 | CF | N | CH | CF | H | CH₂CH₂CH₂CH₂ | NHSO₂N(Me)(CHMe₂) |
| 1-1051 | CCl | N | CH | CF | H | CH₂CH₂CH₂CH₂ | OH |
| 1-1052 | CCl | N | CH | CF | H | CH₂CH₂CH₂CH₂ | OMe |
| 1-1053 | CCl | N | CH | CF | H | CH₂CH₂CH₂CH₂ | OEt |
| 1-1054 | CCl | N | CH | CF | H | CH₂CH₂CH₂CH₂ | OCH₂Ph |
| 1-1055 | CCl | N | CH | CF | H | CH₂CH₂CH₂CH₂ | NHSO₂Me |
| 1-1056 | CCl | N | CH | CF | H | CH₂CH₂CH₂CH₂ | NHSO₂N(Me)(CHMe₂) |
| 1-1057 | CCl | N | CH | CCl | H | CH₂CH₂CH₂CH₂ | OH |
| 1-1058 | CCl | N | CH | CCl | H | CH₂CH₂CH₂CH₂ | OMe |
| 1-1059 | CCl | N | CH | CCl | H | CH₂CH₂CH₂CH₂ | OEt |
| 1-1060 | CCl | N | CH | CCl | H | CH₂CH₂CH₂CH₂ | OCH₂Ph |
| 1-1061 | CCl | N | CH | CCl | H | CH₂CH₂CH₂CH₂ | NHSO₂Me |
| 1-1062 | CCl | N | CH | CCl | H | CH₂CH₂CH₂CH₂ | NHSO₂N(Me)(CHMe₂) |
| 1-1063 | CCF₃ | N | CH | CCl | H | CH₂CH₂CH₂CH₂ | OH |
| 1-1064 | CCF₃ | N | CH | CCl | H | CH₂CH₂CH₂CH₂ | OMe |
| 1-1065 | CCF₃ | N | CH | CCl | H | CH₂CH₂CH₂CH₂ | OEt |
| 1-1066 | CCF₃ | N | CH | CCl | H | CH₂CH₂CH₂CH₂ | OCH₂Ph |
| 1-1067 | CCF₃ | N | CH | CCl | H | CH₂CH₂CH₂CH₂ | NHSO₂Me |
| 1-1068 | CCF₃ | N | CH | CCl | H | CH₂CH₂CH₂CH₂ | NHSO₂N(Me)(CHMe₂) |
| 1-1069 | CCl | N | CH | CBr | H | CH₂CH₂CH₂CH₂ | OH |
| 1-1070 | CCl | N | CH | CBr | H | CH₂CH₂CH₂CH₂ | OMe |
| 1-1071 | CCl | N | CH | CBr | H | CH₂CH₂CH₂CH₂ | OEt |
| 1-1072 | CCl | N | CH | CBr | H | CH₂CH₂CH₂CH₂ | OCH₂Ph |
| 1-1073 | CCl | N | CH | CBr | H | CH₂CH₂CH₂CH₂ | NHSO₂Me |
| 1-1074 | CCl | N | CH | CBr | H | CH₂CH₂CH₂CH₂ | NHSO₂N(Me)(CHMe₂) |
| 1-1075 | CCl | N | CH | CMe | H | CH₂CH₂CH₂CH₂ | OH |
| 1-1076 | CCl | N | CH | CMe | H | CH₂CH₂CH₂CH₂ | OMe |
| 1-1077 | CCl | N | CH | CMe | H | CH₂CH₂CH₂CH₂ | OEt |
| 1-1078 | CCl | N | CH | CMe | H | CH₂CH₂CH₂CH₂ | OCH₂Ph |
| 1-1079 | CCl | N | CH | CMe | H | CH₂CH₂CH₂CH₂ | NHSO₂Me |
| 1-1080 | CCl | N | CH | CMe | H | CH₂CH₂CH₂CH₂ | NHSO₂N(Me)(CHMe₂) |
| 1-1081 | CCl | N | CH | CCF₂H | H | CH₂CH₂CH₂CH₂ | OH |
| 1-1082 | CCl | N | CH | CCF₂H | H | CH₂CH₂CH₂CH₂ | OMe |
| 1-1083 | CCl | N | CH | CCF₂H | H | CH₂CH₂CH₂CH₂ | OEt |
| 1-1084 | CCl | N | CH | CCF₂H | H | CH₂CH₂CH₂CH₂ | OCH₂Ph |
| 1-1085 | CCl | N | CH | CCF₂H | H | CH₂CH₂CH₂CH₂ | NHSO₂Me |
| 1-1086 | CCl | N | CH | CCF₂H | H | CH₂CH₂CH₂CH₂ | NHSO₂N(Me)(CHMe₂) |
| 1-1087 | CCl | N | CH | CCF₂Me | H | CH₂CH₂CH₂CH₂ | OH |
| 1-1088 | CCl | N | CH | CCF₂Me | H | CH₂CH₂CH₂CH₂ | OMe |
| 1-1089 | CCl | N | CH | CCF₂Me | H | CH₂CH₂CH₂CH₂ | OEt |
| 1-1090 | CCl | N | CH | CCF₂Me | H | CH₂CH₂CH₂CH₂ | OCH₂Ph |
| 1-1091 | CCl | N | CH | CCF₂Me | H | CH₂CH₂CH₂CH₂ | NHSO₂Me |
| 1-1092 | CCl | N | CH | CCF₂Me | H | CH₂CH₂CH₂CH₂ | NHSO₂N(Me)(CHMe₂) |
| 1-1093 | CCl | N | CH | CCF₃ | H | CH₂CH₂CH₂CH₂ | OH |
| 1-1094 | CCl | N | CH | CCF₃ | H | CH₂CH₂CH₂CH₂ | OMe |
| 1-1095 | CCl | N | CH | CCF₃ | H | CH₂CH₂CH₂CH₂ | OEt |
| 1-1096 | CCl | N | CH | CCF₃ | H | CH₂CH₂CH₂CH₂ | OCH₂Ph |
| 1-1097 | CCl | N | CH | CCF₃ | H | CH₂CH₂CH₂CH₂ | NHSO₂Me |
| 1-1098 | CCl | N | CH | CCF₃ | H | CH₂CH₂CH₂CH₂ | NHSO₂N(Me)(CHMe₂) |
| 1-1099 | CH | N | CH | CCF₃ | H | CH₂CH₂CH₂CH₂ | OH |
| 1-1100 | CH | N | CH | CCF₃ | H | CH₂CH₂CH₂CH₂ | OMe |
| 1-1101 | CH | N | CH | CCF₃ | H | CH₂CH₂CH₂CH₂ | OEt |
| 1-1102 | CH | N | CH | CCF₃ | H | CH₂CH₂CH₂CH₂ | OCH₂Ph |
| 1-1103 | CH | N | CH | CCF₃ | H | CH₂CH₂CH₂CH₂ | NHSO₂Me |
| 1-1104 | CH | N | CH | CCF₃ | H | CH₂CH₂CH₂CH₂ | NHSO₂N(Me)(CHMe₂) |
| 1-1105 | CF | N | CH | CCF₃ | H | CH₂CH₂CH₂CH₂ | OH |
| 1-1106 | CF | N | CH | CCF₃ | H | CH₂CH₂CH₂CH₂ | OMe |
| 1-1107 | CF | N | CH | CCF₃ | H | CH₂CH₂CH₂CH₂ | OEt |
| 1-1108 | CF | N | CH | CCF₃ | H | CH₂CH₂CH₂CH₂ | OCH₂Ph |
| 1-1109 | CF | N | CH | CCF₃ | H | CH₂CH₂CH₂CH₂ | NHSO₂Me |
| 1-1110 | CF | N | CH | CCF₃ | H | CH₂CH₂CH₂CH₂ | NHSO₂N(Me)(CHMe₂) |
| 1-1111 | CBr | N | CH | CCF₃ | H | CH₂CH₂CH₂CH₂ | OH |
| 1-1112 | CBr | N | CH | CCF₃ | H | CH₂CH₂CH₂CH₂ | OMe |
| 1-1113 | CBr | N | CH | CCF₃ | H | CH₂CH₂CH₂CH₂ | OEt |
| 1-1114 | CBr | N | CH | CCF₃ | H | CH₂CH₂CH₂CH₂ | OCH₂Ph |
| 1-1115 | CBr | N | CH | CCF₃ | H | CH₂CH₂CH₂CH₂ | NHSO₂Me |
| 1-1116 | CBr | N | CH | CCF₃ | H | CH₂CH₂CH₂CH₂ | NHSO₂N(Me)(CHMe₂) |
| 1-1117 | CCl | N | CH | CSO₂Me | H | CH₂CH₂CH₂CH₂ | OH |
| 1-1118 | CCl | N | CH | CSO₂Me | H | CH₂CH₂CH₂CH₂ | OMe |
| 1-1119 | CCl | N | CH | CSO₂Me | H | CH₂CH₂CH₂CH₂ | OEt |
| 1-1120 | CCl | N | CH | CSO₂Me | H | CH₂CH₂CH₂CH₂ | OCH₂Ph |
| 1-1121 | CCl | N | CH | CSO₂Me | H | CH₂CH₂CH₂CH₂ | NHSO₂Me |
| 1-1122 | CCl | N | CH | CSO₂Me | H | CH₂CH₂CH₂CH₂ | NHSO₂N(Me)(CHMe₂) |
| 1-1123 | CH | CH | N | COCF₂O | | CH₂CH₂CH₂CH₂ | OH |
| 1-1124 | CH | CH | N | COCF₂O | | CH₂CH₂CH₂CH₂ | OMe |
| 1-1125 | CH | CH | N | COCF₂O | | CH₂CH₂CH₂CH₂ | OEt |
| 1-1126 | CH | CH | N | COCF₂O | | CH₂CH₂CH₂CH₂ | OCH₂Ph |
| 1-1127 | CH | CH | N | COCF₂O | | CH₂CH₂CH₂CH₂ | NHSO₂Me |
| 1-1128 | CH | CH | N | COCF₂O | | CH₂CH₂CH₂CH₂ | NHSO₂N(Me)(CHMe₂) |
| 1-1129 | CH | F | N | CH | CF₃ | CH₂CH₂CH₂CH₂ | OH |
| 1-1130 | CH | F | N | CH | CF₃ | CH₂CH₂CH₂CH₂ | OMe |
| 1-1131 | CH | F | N | CH | CF₃ | CH₂CH₂CH₂CH₂ | OEt |
| 1-1132 | CH | F | N | CH | CF₃ | CH₂CH₂CH₂CH₂ | OCH₂Ph |
| 1-1133 | CH | F | N | CH | CF₃ | CH₂CH₂CH₂CH₂ | NHSO₂Me |
| 1-1134 | CH | F | N | CH | CF₃ | CH₂CH₂CH₂CH₂ | NHSO₂N(Me)(CHMe₂) |
| 1-1135 | CH | CCl | CCl | N | H | CH₂CH₂CH₂CH₂ | OH |
| 1-1136 | CH | CCl | CCl | N | H | CH₂CH₂CH₂CH₂ | OMe |
| 1-1137 | CH | CCl | CCl | N | H | CH₂CH₂CH₂CH₂ | OEt |
| 1-1138 | CH | CCl | CCl | N | H | CH₂CH₂CH₂CH₂ | OCH₂Ph |
| 1-1139 | CH | CCl | CCl | N | H | CH₂CH₂CH₂CH₂ | NHSO₂Me |
| 1-1140 | CH | CCl | CCl | N | H | CH₂CH₂CH₂CH₂ | NHSO₂N(Me)(CHMe₂) |

1140 compounds of formula (I), wherein R³ is fluorine, R⁴ is chlorine, Y is C—H and B is oxygen, and the values of X¹, X², X³, X⁴, R², D and R⁸ are as given in Table 1 for compounds 1-1 to 1-1140, are designated as compound numbers 2-1 to 2-1140 respectively.

1140 compounds of formula (I), wherein R³ is chlorine, R⁴ is chlorine, Y is C—H and B is oxygen, and the values of X¹, X², X³, X⁴, R², D and R⁸ are as given in Table 1 for compounds 1-1 to 1-1140, are designated as compound numbers 3-1 to 3-1140 respectively.

1140 compounds of formula (I), wherein R³ is hydrogen, R⁴ is bromine, Y is C—H and B is oxygen, and the values of X¹, X², X³, X⁴, R², D and R⁸ are as given in Table 1 for compounds 1-1 to 1-1140, are designated as compound numbers 4-1 to 4-1140 respectively.

1140 compounds of formula (I), wherein R³ is fluorine, R⁴ is bromine, Y is C—H and B is oxygen, and the values of X¹, X², X³, X⁴, R², D and R⁸ are as given in Table 1 for compounds 1-1 to 1-1140, are designated as compound numbers 5-1 to 5-1140 respectively.

1140 compounds of formula (I), wherein R³ is chlorine, R⁴ is bromine, Y is C—H and B is oxygen, and the values of X¹, X², X³, X⁴, R², D and R⁸ are as given in Table 1 for compounds 1-1 to 1-1140, are designated as compound numbers 6-1 to 6-1140 respectively.

1140 compounds of formula (I), wherein R³ is hydrogen, R⁴ is cyano, Y is C—H and B is oxygen, and the values of X¹, X², X³, X⁴, R², D and R⁸ are as given in Table 1 for compounds 1-1 to 1-1140, are designated as compound numbers 7-1 to 7-1140 respectively.

1140 compounds of formula (I), wherein R³ is fluorine, R⁴ is cyano, Y is C—H and B is oxygen, and the values of X¹, X², X³, X⁴, R², D and R⁸ are as given in Table 1 for compounds 1-1 to 1-1140, are designated as compound numbers 8-1 to 8-1140 respectively.

1140 compounds of formula (I), wherein R³ is chlorine, R⁴ is cyano, Y is C—H and B is oxygen, and the values of X¹, X², X³, X⁴, R², D and R⁸ are as given in Table 1 for compounds 1-1 to 1-1140, are designated as compound numbers 9-1 to 9-1140 respectively.

1140 compounds of formula (I), wherein R³ is hydrogen, R⁴ is chlorine, Y is nitrogen and B is oxygen, and the values of X¹, X², X³, X⁴, R², D and R⁸ are as given in Table 1 for compounds 1-1 to 1-1140, are designated as compound numbers 10-1 to 10-1140 respectively.

1140 compounds of formula (I), wherein R³ is fluorine, R⁴ is chlorine, Y is nitrogen and B is oxygen, and the values of X¹, X², X³, X⁴, R², D and R⁸ are as given in Table 1 for compounds 1-1 to 1-1140, are designated as compound numbers 11-1 to 11-1140 respectively.

1140 compounds of formula (I), wherein R³ is chlorine, R⁴ is chlorine, Y is nitrogen and B is oxygen, and the values of X¹, X², X³, X⁴, R², D and R⁸ are as given in Table 1 for compounds 1-1 to 1-1140, are designated as compound numbers 12-1 to 12-1140 respectively.

1140 compounds of formula (I), wherein R³ is hydrogen, R⁴ is bromine, Y is nitrogen and B is oxygen, and the values of X¹, X², X³, X⁴, R², D and R⁸ are as given in Table 1 for compounds 1-1 to 1-1140, are designated as compound numbers 13-1 to 13-1140 respectively.

1140 compounds of formula (I), wherein R³ is fluorine, R⁴ is bromine, Y is nitrogen and B is oxygen, and the values of X¹, X², X³, X⁴, R², D and R⁸ are as given in Table 1 for compounds 1-1 to 1-1140, are designated as compound numbers 14-1 to 14-1140 respectively.

1140 compounds of formula (I), wherein R³ is chlorine, R⁴ is bromine, Y is nitrogen and B is oxygen, and the values of X¹, X², X³, X⁴, R², D and R⁸ are as given in Table 1 for compounds 1-1 to 1-1140, are designated as compound numbers 15-1 to 15-1140 respectively.

1140 compounds of formula (I), wherein R³ is hydrogen, R⁴ is cyano, Y is nitrogen and B is oxygen, and the values of X¹, X², X³, X⁴, R², D and R⁸ are as given in Table 1 for compounds 1-1 to 1-1140, are designated as compound numbers 16-1 to 16-1140 respectively.

1140 compounds of formula (I), wherein R³ is fluorine, R⁴ is cyano, Y is nitrogen and B is oxygen, and the values of X¹, X², X³, X⁴, R², D and R⁸ are as given in Table 1 for compounds 1-1 to 1-1140, are designated as compound numbers 17-1 to 17-1140 respectively.

1140 compounds of formula (I), wherein R³ is chlorine, R⁴ is cyano, Y is nitrogen and B is oxygen, and the values of X¹, X², X³, X⁴, R², D and R⁸ are as given in Table 1 for compounds 1-1 to 1-1140, are designated as compound numbers 18-1 to 18-1140 respectively.

1140 compounds of formula (I), wherein R³ is hydrogen, R⁴ is chlorine, Y is C—H and B is N—H, and the values of X¹, X², X³, X⁴, R², D and R⁸ are as given in Table 1 for compounds 1-1 to 1-1140, are designated as compound numbers 19-1 to 19-1140 respectively.

1140 compounds of formula (I), wherein R³ is fluorine, R⁴ is chlorine, Y is C—H and B is N—H, and the values of X¹, X², X³, X⁴, R², D and R⁸ are as given in Table 1 for compounds 1-1 to 1-1140, are designated as compound numbers 20-1 to 20-1140 respectively.

1140 compounds of formula (I), wherein R³ is chlorine, R⁴ is chlorine, Y is C—H and B is N—H, and the values of X¹, X², X³, X⁴, R², D and R⁸ are as given in Table 1 for compounds 1-1 to 1-1140, are designated as compound numbers 21-1 to 21-1140 respectively.

1140 compounds of formula (I), wherein R³ is hydrogen, R⁴ is bromine, Y is C—H and B is N—H, and the values of X¹, X², X³, X⁴, R², D and R⁸ are as given in Table 1 for compounds 1-1 to 1-1140, are designated as compound numbers 22-1 to 22-1140 respectively.

1140 compounds of formula (I), wherein R³ is fluorine, R⁴ is bromine, Y is C—H and B is N—H, and the values of X¹, X², X³, X⁴, R², D and R⁸ are as given in Table 1 for compounds 1-1 to 1-1140, are designated as compound numbers 23-1 to 23-1140 respectively.

1140 compounds of formula (I), wherein R³ is chlorine, R⁴ is bromine, Y is C—H and B is N—H, and the values of X¹, X², X³, X⁴, R², D and R⁸ are as given in Table 1 for compounds 1-1 to 1-1140, are designated as compound numbers 24-1 to 24-1140 respectively.

1140 compounds of formula (I), wherein R³ is hydrogen, R⁴ is cyano, Y is C—H and B is N—H, and the values of X¹, X², X³, X⁴, R², D and R⁸ are as given in Table 1 for compounds 1-1 to 1-1140, are designated as compound numbers 25-1 to 25-1140 respectively.

1140 compounds of formula (I), wherein R³ is fluorine, R⁴ is cyano, Y is C—H and B is N—H, and the values of X¹, X², X³, X⁴, R², D and R⁸ are as given in Table 1 for compounds 1-1 to 1-1140, are designated as compound numbers 26-1 to 26-1140 respectively.

1140 compounds of formula (I), wherein R³ is chlorine, R⁴ is cyano, Y is C—H and B is N—H, and the values of X¹, X², X³, X⁴, R², D and R⁸ are as given in Table 1 for compounds 1-1 to 1-1140, are designated as compound numbers 27-1 to 27-1140 respectively.

1140 compounds of formula (I), wherein R³ is hydrogen, R⁴ is chlorine, Y is nitrogen and B is N—H, and the values of X¹, X², X³, X⁴, R², D and R⁸ are as given in Table 1 for compounds 1-1 to 1-1140, are designated as compound numbers 28-1 to 28-1140 respectively.

1140 compounds of formula (I), wherein R³ is fluorine, R⁴ is chlorine, Y is nitrogen and B is N—H, and the values of X¹, X², X³, X⁴, R², D and R⁸ are as given in Table 1 for compounds 1-1 to 1-1140, are designated as compound numbers 29-1 to 29-1140 respectively.

1140 compounds of formula (I), wherein R³ is chlorine, R⁴ is chlorine, Y is nitrogen and B is N—H, and the values of X¹, X², X³, X⁴, R², D and R⁸ are as given in Table 1 for compounds 1-1 to 1-1140, are designated as compound numbers 30-1 to 30-1140 respectively.

1140 compounds of formula (I), wherein R³ is hydrogen, R⁴ is bromine, Y is nitrogen and B is N—H, and the values of X¹, X², X³, X⁴, R², D and R⁸ are as given in Table 1 for compounds 1-1 to 1-1140, are designated as compound numbers 31-1 to 31-1140 respectively.

1140 compounds of formula (I), wherein R³ is fluorine, R⁴ is bromine, Y is nitrogen and B is N—H, and the values of X¹, X², X³, X⁴, R², D and R⁸ are as given in Table 1 for compounds 1-1 to 1-1140, are designated as compound numbers 32-1 to 32-1140 respectively.

1140 compounds of formula (I), wherein R³ is chlorine, R⁴ is bromine, Y is nitrogen and B is N—H, and the values of X¹, X², X³, X⁴, R², D and R⁸ are as given in Table 1 for compounds 1-1 to 1-1140, are designated as compound numbers 33-1 to 33-1140 respectively.

1140 compounds of formula (I), wherein R³ is hydrogen, R⁴ is cyano, Y is nitrogen and B is N—H, and the values of X¹, X², X³, X⁴, R², D and R⁸ are as given in Table 1 for compounds 1-1 to 1-1140, are designated as compound numbers 34-1 to 34-1140 respectively.

1140 compounds of formula (I), wherein R³ is fluorine, R⁴ is cyano, Y is nitrogen and B is N—H, and the values of X¹, X², X³, X⁴, R², D and R⁸ are as given in Table 1 for compounds 1-1 to 1-1140, are designated as compound numbers 35-1 to 35-1140 respectively.

1140 compounds of formula (I), wherein R³ is chlorine, R⁴ is cyano, Y is nitrogen and B is N—H, and the values of X¹, X², X³, X⁴, R², D and R⁸ are as given in Table 1 for compounds 1-1 to 1-1140, are designated as compound numbers 36-1 to 36-1140 respectively.

Compounds of the invention may be prepared by techniques known to the person skilled in the art of organic chemistry. General methods for the production of compounds of formula (I) are described below. Unless otherwise stated in the text, the substituents X¹, X², X³, X⁴, Y, B, D, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

Compounds of formula (I) may be prepared from compounds of formula (A) and compounds of formula (B) as shown in reaction scheme 1.

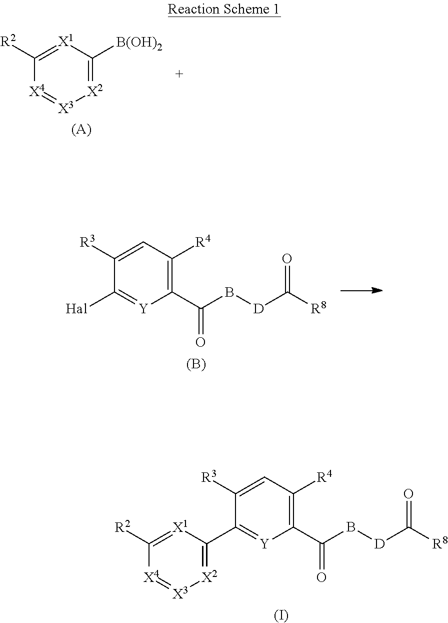

For example, a mixture of a compound of formula (A) and a compound of formula (B), wherein Hal represents a halogen atom, for example a chlorine, bromine or iodine atom, may be treated with a metal catalyst, such as palladium acetate, optionally in the presence of a suitable ligand, such as a phosphine ligand, for example S-Phos, or a preformed complex of a metal and a ligand, such as dppf palladium dichloride, and a base, such as potassium acetate, in a suitable solvent such as dioxane. Boronic acids (or the corresponding boronate esters) of formula (A) are available or may be prepared by methods well known in the literature. Compounds of formula (B) may be prepared from anilines of formula (C) as shown in reaction scheme 2.

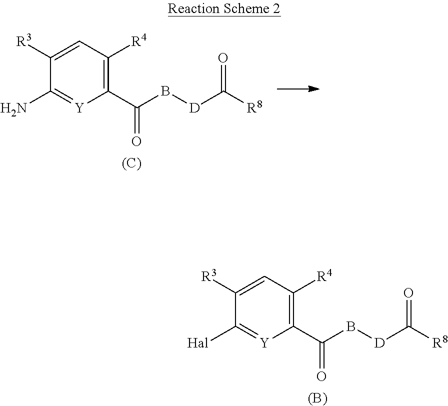

For example, a compound of formula (C) may be treated with a metal halide, such as potassium iodide, and a nitrosylating reagent, such as sodium nitrite and toluene sulphonic acid, in a suitable solvent, such as a mixture of water and acetonitrile. Anilines of formula (C) may be prepared from nitro compounds of formula (D) as shown in reaction scheme 3.

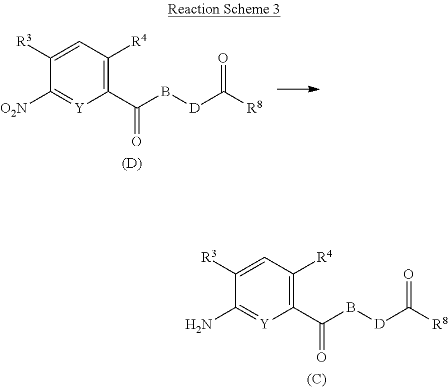

For example, a compound of formula (D) can be treated with a reducing agent, such as iron and ammonium chloride, in a suitable solvent, such as a mixture of water and ethanol. Nitro compounds of formula (D) may be prepared from acids of formula (E) and alcohols or amines of formula (F) as shown in reaction scheme 4.

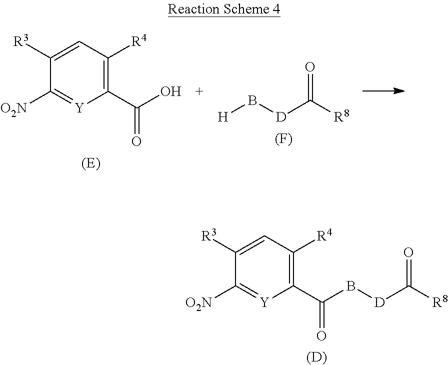

For example, an acid of formula (E) may be treated with an activating agent such as oxalyl chloride in a suitable solvent, such as dichloromethane and dimethylformamide, and the resulting intermediate then treated with an alcohol or amine of formula (F) in the presence of a base, such as triethylamine, in a suitable solvent such as dichloromethane. Alcohols and amines of formula (F) are available or may be prepared by methods well known in the literature. Acids of formula (E) are available or may be prepared by methods well known in the literature. Alternatively compounds of formula (I) may be prepared from alcohols or amines of formula (F) and acids of formula (G) as shown in reaction scheme 5.

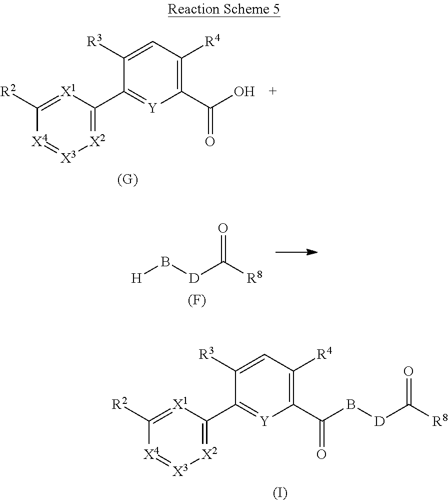

For example, an acid of formula (G) may be treated with an activating agent such as oxalyl chloride in a suitable solvent, such as dichloromethane and dimethylformamide, and the resulting intermediate then treated with an alcohol or amine of formula (F) in the presence of a base, such as triethylamine, in a suitable solvent such as dichloromethane. Acids of formula (G) may be prepared from esters of formula (H) as shown in reaction scheme 6.

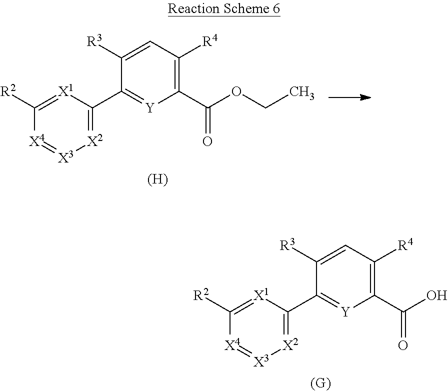

For example, an ester of formula (H) may be treated with sodium hydroxide in a suitable solvent, such as a mixture of water and ethanol. Esters of formula (H) can be prepared from compounds of formula (J) and compounds of formula (A) as shown in reaction scheme 7.

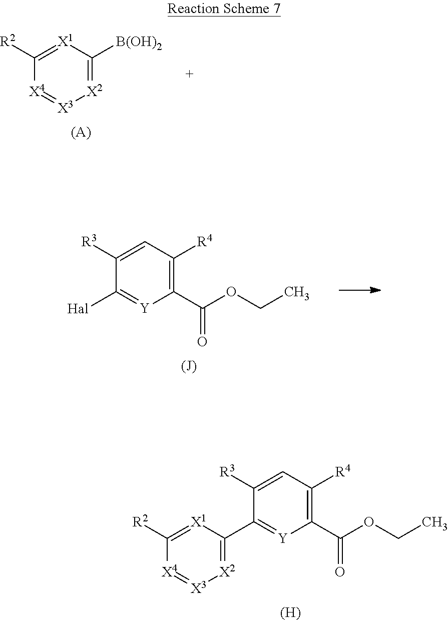

For example, a mixture of a compound of formula (A) and a compound of formula (J), wherein Hal represents a halogen atom, for example a chlorine, bromine or iodine atom, may be treated with a metal catalyst, such as palladium acetate, optionally in the presence of a suitable ligand, such as a phosphine ligand, for example S-Phos, or a preformed complex of a metal and a ligand, such as dppf palladium dichloride, and a base, such as potassium acetate, in a suitable solvent such as dioxane. Boronic acids (or the corresponding boronate esters) of formula (A) are available or may be prepared by methods well known in the literature. Compounds of formula (J) may be prepared from anilines of formula (K) as shown in reaction scheme 8.

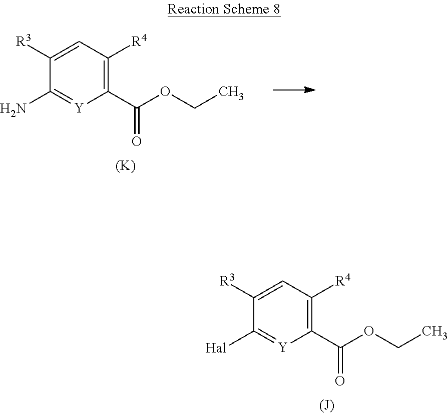

For example, a compound of formula (K) may be treated with a metal halide, such as potassium iodide, and a nitrosylating reagent, such as sodium nitrite and toluene sulphonic acid, in a suitable solvent, such as a mixture of water and acetonitrile. Anilines of formula (K) are available or may be prepared from nitro compounds of formula (L) as shown in reaction scheme 9.

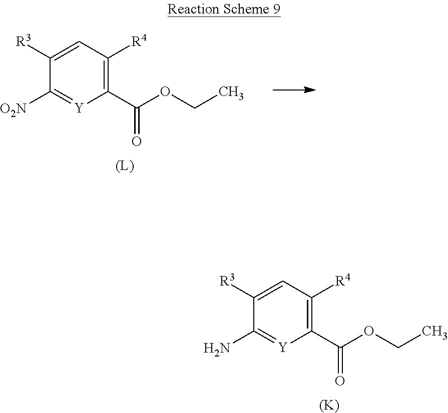

For example, a compound of formula (L) can be treated with a reducing agent, such as iron and ammonium chloride, in a suitable solvent, such as a mixture of water and ethanol. Nitro compounds of formula (L) are available or may be prepared by methods well known in the literature. Compounds of formula (I-A), which are compounds of formula (I) in which R⁸ is an OH group, may be prepared from compounds of formula (I-B), which are compounds of formula (I) in which R⁸ is OR⁹, as shown in reaction scheme 10.

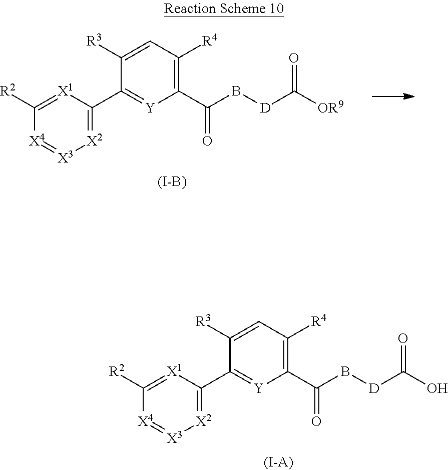

For example, a compound of formula (I-B) may be treated with hydrochloric acid in a suitable solvent, such as dioxane. Compounds of formula (I-C), which are compounds of formula (I) in which R⁸ is NR¹⁰R¹¹, may be prepared from compounds of formula (I-A) as shown in reaction scheme 11.

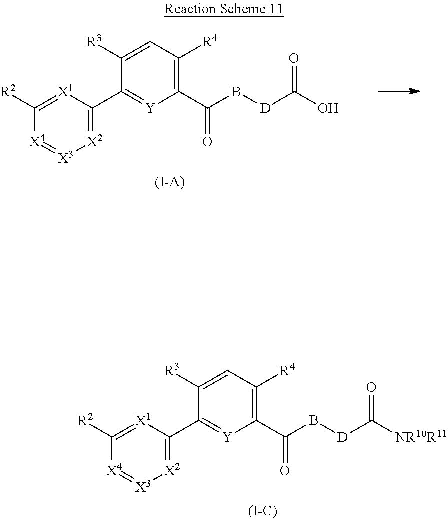

For example, a compound of formula (I-A) may be treated with a halogenating reagent, such as oxalyl chloride, in a suitable solvent, such as dichloromethane, to form an acyl halide which may be treated with a reagent HNR¹⁰R¹¹ in the presence of a base, such as triethylamine, in a suitable solvent, such as dichloromethane.

One skilled in the art will realise that it is often possible to alter the order in which the transformations described above are conducted, or to combine them in alternative ways to prepare a wide range of compounds of formula (I). Multiple steps may also be combined in a single reaction. All such variations are contemplated within the scope of the invention.

The skilled person will also be aware that some reagents will be incompatible with certain values or combinations of the substituents X¹, X², X³, X⁴, Y, B, D, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ as defined herein, and any additional steps, such as protection and/or deprotection steps, which are necessary to achieve the desired transformation will be clear to the skilled person.

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). For water-soluble compounds, soluble liquids, water-soluble concentrates or water soluble granules are preferred. Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha. Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of formula (I) can be used in combination with one or more other herbicides to provide various herbicidal mixtures. Specific examples of such mixtures include (wherein “I” represents a compound of formula (I)): —I+acetochlor; I+acifluorfen (including acifluorfen-sodium); I+aclonifen; I+alachlor; I+alloxydim; I+ametryn; I+amicarbazone; I+amidosulfuron; I+aminocyclopyrachlor; I+aminopyralid; I+amitrole; I+asulam; I+atrazine; I+bensulfuron (including bensulfuron-methyl); I+bentazone; I+bicyclopyrone; I+bilanafos; I+bifenox; I+bispyribac-sodium; I+bixlozone; I+bromacil; I+bromoxynil; I+butachlor; I+butafenacil; I+cafenstrole; I+carfentrazone (including carfentrazone-ethyl); cloransulam (including cloransulam-methyl); I+chlorimuron (including chlorimuron-ethyl); I+chlorotoluron; I+cinosulfuron; I+chlorsulfuron; I+cinmethylin; I+clacyfos; I+clethodim; I+clodinafop (including clodinafop-propargyl); I+clomazone; I+clopyralid; I+cyclopyranil; I+cyclopyrimorate; I+cyclosulfamuron; I+cyhalofop (including cyhalofop-butyl); I+2,4-D (including the choline salt and 2-ethylhexyl ester thereof; I+2,4-DB; I+daimuron; I+desmedipham; I+dicamba (including the aluminum, aminopropyl, bis-aminopropylmethyl, choline, dichloroprop, diglycolamine, dimethylamine, dimethylammonium, potassium and sodium salts thereof); I+diclofop-methyl; I+diclosulam; I+diflufenican; I+difenzoquat; I+diflufenican; I+diflufenzopyr; I+dimethachlor; I+dimethenamid-P; I+diquat dibromide; I+diuron; I+esprocarb; I+ethalfluralin; I+ethofumesate; I+fenoxaprop (including fenoxaprop-P-ethyl); I+fenoxasulfone; I+fenquinotrione; I+fentrazamide; I+flazasulfuron; I+florasulam; I+florpyrauxifen; I+fluazifop (including fluazifop-P-butyl); I+flucarbazone (including flucarbazone-sodium); I+flufenacet; I+flumetralin; I+flumetsulam; I+flumioxazin; I+flupyrsulfuron (including flupyrsulfuron-methyl-sodium); I+fluroxypyr (including fluroxypyr-meptyl); I+fluthiacet-methyl; I+fomesafen; I+foramsulfuron; I+glufosinate (including the ammonium salt thereof); I+glyphosate (including the diammonium, isopropylammonium and potassium salts thereof); I+halauxifen (including halauxifen-methyl); I+halosulfuron-methyl; I+haloxyfop (including haloxyfop-methyl); I+hexazinone; I+hydantocidin; I+imazamox; I+imazapic; I+imazapyr; I+imazaquin; I+imazethapyr; I+indaziflam; I+iodosulfuron (including iodosulfuron-methyl-sodium); I+iofensulfuron; I+iofensulfuron-sodium; I+ioxynil; I+ipfencarbazone; I+isoproturon; I+isoxaben; I+isoxaflutole; I+lactofen; I+lancotrione; I+linuron; I+MCPA; I+MCPB; I+mecoprop-P; I+mefenacet; I+mesosulfuron; I+mesosulfuron-methyl; I+mesotrione; I+metamitron; I+metazachlor; I+methiozolin; I+metobromuron; I+metolachlor; I+metosulam; I+metoxuron; I+metribuzin; I+metsulfuron; I+molinate; I+napropamide; I+nicosulfuron; I+norflurazon; I+orthosulfamuron; I+oxadiargyl; I+oxadiazon; I+oxasulfuron; I+oxyfluorfen; I+paraquat dichloride; I+pendimethalin; I+penoxsulam; I+phenmedipham; I+picloram; I+picolinafen; I+pinoxaden; I+pretilachlor; I+primisulfuron-methyl; I+prodiamine; I+prometryn; I+propachlor; I+propanil; I+propaquizafop; I+propham; I+propyrisulfuron, I+propyzamide; I+prosulfocarb; I+prosulfuron; I+pyraclonil; I+pyraflufen (including pyraflufen-ethyl): I+pyrasulfotole; I+pyrazolynate, I+pyrazosulfuron-ethyl; I+pyribenzoxim; I+pyridate; I+pyriftalid; I+pyrimisulfan, I+pyrithiobac-sodium; I+pyroxasulfone; I+pyroxsulam; I+quinclorac; I+quinmerac; I+quizalofop (including quizalofop-P-ethyl and quizalofop-P-tefuryl); I+rimsulfuron; I+saflufenacil; I+sethoxydim; I+simazine; I+S-metolachlor; I+sulcotrione; I+sulfentrazone; I+sulfosulfuron; I+tebuthiuron; I+tefuryltrione; I+tembotrione; I+terbuthylazine; I+terbutryn; I+thiencarbazone; I+thifensulfuron; I+tiafenacil; I+tolpyralate; I+topramezone; I+tralkoxydim; I+triafamone; I+triallate; I+triasulfuron; I+tribenuron (including tribenuron-methyl); I+triclopyr; I+trifloxysulfuron (including trifloxysulfuron-sodium); I+trifludimoxazin; I+trifluralin; I+triflusulfuron; I+tritosulfuron; I+4-hydroxy-1-methoxy-5-methyl-3-[4-(trifluoromethyl)-2-pyridyl]imidazolidin-2-one; I+4-hydroxy-1,5-dimethyl-3-[4-(trifluoromethyl)-2-pyridyl]imidazolidin-2-one; I+5-ethoxy-4-hydroxy-1-methyl-3-[4-(trifluoromethyl)-2-pyridyl]imidazolidin-2-one; I+4-hydroxy-1-methyl-3-[4-(trifluoromethyl)-2-pyridyl]imidazolidin-2-one; I+4-hydroxy-1,5-dimethyl-3-[1-methyl-5-(trifluoromethyl)pyrazol-3-yl]imidazolidin-2-one; I+(4R)1-(5-tert-butylisoxazol-3-yl)-4-ethoxy-5-hydroxy-3-methyl-imidazolidin-2-one; I+3-[2-(3,4-dimethoxyphenyl)-6-methyl-3-oxo-pyridazine-4-carbonyl]bicyclo[3.2.1]octane-2,4-dione; I+2-[2-(3,4-dimethoxyphenyl)-6-methyl-3-oxo-pyridazine-4-carbonyl]-5-methyl-cyclohexane-1,3-dione; I+2-[2-(3,4-dimethoxyphenyl)-6-methyl-3-oxo-pyridazine-4-carbonyl]cyclohexane-1,3-dione; I+2-[2-(3,4-dimethoxyphenyl)-6-methyl-3-oxo-pyridazine-4-carbonyl]-5,5-dimethyl-cyclohexane-1,3-dione; I+6-[2-(3,4-dimethoxyphenyl)-6-methyl-3-oxo-pyridazine-4-carbonyl]-2,2,4,4-tetramethyl-cyclohexane-1,3,5-trione; I+2-[2-(3,4-dimethoxyphenyl)-6-methyl-3-oxo-pyridazine-4-carbonyl]-5-ethyl-cyclohexane-1,3-dione; I+2-[2-(3,4-dimethoxyphenyl)-6-methyl-3-oxo-pyridazine-4-carbonyl]-4,4,6,6-tetramethyl-cyclohexane-1,3-dione; I+2-[6-cyclopropyl-2-(3,4-dimethoxyphenyl)-3-oxo-pyridazine-4-carbonyl]-5-methyl-cyclohexane-1,3-dione; I+3-[6-cyclopropyl-2-(3,4-dimethoxyphenyl)-3-oxo-pyridazine-4-carbonyl]bicyclo[3.2.1]octane-2,4-dione; I+2-[6-cyclopropyl-2-(3,4-dimethoxyphenyl)-3-oxo-pyridazine-4-carbonyl]-5,5-dimethyl-cyclohexane-1,3-dione; I+6-[6-cyclopropyl-2-(3,4-dimethoxyphenyl)-3-oxo-pyridazine-4-carbonyl]-2,2,4,4-tetramethyl-cyclohexane-1,3,5-trione; I+2-[6-cyclopropyl-2-(3,4-dimethoxyphenyl)-3-oxo-pyridazine-4-carbonyl]cyclohexane-1,3-dione; I+4-[2-(3,4-dimethoxyphenyl)-6-methyl-3-oxo-pyridazine-4-carbonyl]-2,2,6,6-tetramethyl-tetrahydropyran-3,5-dione and I+4-[6-cyclopropyl-2-(3,4-dimethoxyphenyl)-3-oxo-pyridazine-4-carbonyl]-2,2,6,6-tetramethyl-tetrahydropyran-3,5-dione.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Preferred combinations (wherein “I” represents a compound of formula (I)) include: —I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; I+furilazole I+isoxadifen (including isoxadifen-ethyl); I+mefenpyr (including mefenpyr-diethyl); I+metcamifen; I+N-(2-methoxybenzoyl)-4-[(methylaminocarbonyl)amino]benzenesulfonamide and I+oxabetrinil.

Particularly preferred are mixtures of a compound of formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha. A preferred range is 10-200 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

The compounds of the present invention can be used in methods of controlling undesired vegetation in crop plants which are tolerant to protoporphyrinogen oxidase (PPO) inhibitors. Such plants can be obtained, for example, by transforming crop plants with nucleic acids which encode a suitable protoporphyrinogen oxidase, which may contain a mutation in order to make it more resistant to the PPO inhibitor. Examples of such nucleic acids and crop plants are disclosed in WO95/34659, WO97/32011, WO2007/024739, WO2012/080975, WO2013/189984, WO2015/022636, WO2015/022640, WO2015/092706, WO2016/099153, WO2017/023778, WO2017/039969, WO2017/217793, WO2017/217794, WO2018/114759, WO2019/117578, WO2019/117579 and WO2019/118726. Thus the present invention also provides a method for controlling undesired vegetation at a plant cultivation site, the method comprising the steps of: a) providing, at said site, a plant that comprises at least one nucleic acid comprising a nucleotide sequence encoding a protoporphyrinogen oxidase (PPO) polypeptide which is resistant or tolerant to a “PPO inhibiting herbicide”; b) applying to said site an effective amount of said herbicide, wherein the PPO inhibiting herbicide is a compound of formula (I) as herein defined.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

Unwanted plants are to be understood as also including those weeds that have been rendered tolerant to herbicides or classes of herbicides (e.g. ALS-, GS-, EPSPS-, PPO-, ACCase- and HPPD-inhibitors) by evolution, by conventional methods of breeding or by genetic engineering. Examples include Amaranthus palmeri that has evolved resistance to glyphosate and/or acetolactate synthase (ALS) inhibiting herbicides.

The compounds of the present invention can be used in methods of controlling unwanted plants or weeds which are resistant to protoporphyrinogen oxidase (PPO) inhibitors. For example, Amaranthus palmeri and Amaranthus tuberculatus populations have evolved as PPO-resistant weeds e.g. due to amino acid substitutions in PPX2L such as those occurring at amino acids R128 (also referred to as R98) and G399, or a codon (glycine) deletion in PPX2L at codon 210 (Δ210), the codon numbering being based on NCBI reference DQ386114. The compounds of the present invention can be used in methods of controlling Amaranthus palmeri and/or Amaranthus tuberculatus with mutations or deletions at the previously mentioned codons or equivalents, and it would be obvious to try the compounds to control unwanted plants or weeds with other mutations conferring tolerance or resistance to PPO inhibitors that may arise.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Synthesis Examples

### Example 1 Preparation of [(1S)-3-ethoxy-1-methyl-3-oxo-propyl] 2-chloro-5-[3-chloro-5-(trifluoromethyl)-2-pyridyl]-4-fluoro-benzoate (Compound 2-525; S-enantiomer)

### Step 1: Synthesis of 5-bromo-2-chloro-4-fluoro-benzoic acid

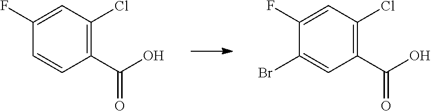 [SMILES: O=C(O)c1ccc(F)cc1Cl ; O=C(O)c1cc(Br)c(F)cc1Cl]

Sulphur (150 mg, 4.6 mmol) and bromine (0.5 ml, 9.8 mmol) were added to a stirred solution of 2-chloro-4-fluoro-benzoic acid (3.4 g, 19 mmol) in chlorosulphonic acid (15 ml) at ambient temperature. The resulting mixture was heated at 70° C. for 4 hours, then cooled and added slowly to iced water (300 ml). The resulting mixture was extracted with ethyl acetate (200 ml) and the organic extract was dried over magnesium sulfate, filtered and evaporated under reduced pressure to provide 5-bromo-2-chloro-4-fluoro-benzoic acid as a white solid (4.7 g). ¹H NMR (400 MHz, CDCl₃) δ 7.3 (d, 1H), 7.25 (dd, 1H ppm (acid proton not observed).

### Step 2: Synthesis of [(1S)-3-ethoxy-1-methyl-3-oxo-propyl] 5-bromo-2-chloro-4-fluoro-benzoate

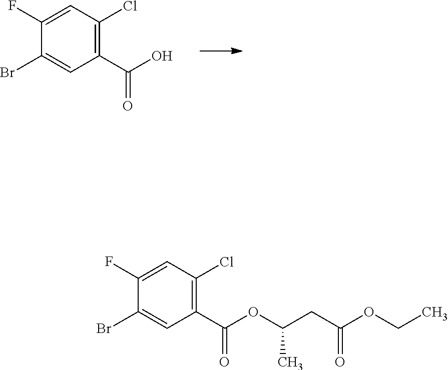 [SMILES: O=C(O)c1cc(Br)c(F)cc1Cl ; CCOC(=O)C[C@H](C)OC(=O)c1cc(Br)c(F)cc1Cl]

Oxalyl chloride (0.45 ml, 5.1 mmol) was added dropwise to a stirred solution of 5-bromo-2-chloro-4-fluoro-benzoic acid (0.90 g, 3.4 mmol) in dichloromethane (20 ml) at ambient temperature. The resulting solution was stirred at room temperature for 5 mins, then dimethylformamide (5 drops) was added and the resulting solution stirred for a further 60 mins. The solvent was evaporated under reduced pressure and the residue dissolved in dichloromethane (20 ml). To this solution was added a solution of triethylamine (0.52 ml, 3.7 mmol) and ethyl (3S)-3-hydroxybutanoate (0.49 g, 3.7 mmol) in dichloromethane (5 ml) and the resulting mixture stirred at ambient temperature for 1 hour, then evaporated under reduced pressure and the residue purified by chromatography to provide [(1S)-3-ethoxy-1-methyl-3-oxo-propyl] 5-bromo-2-chloro-4-fluoro-benzoate as an oil (1.1 g).

Also prepared by this general method were:
[(1S)-3-ethoxy-1-methyl-3-oxo-propyl] 5-bromo-2-chloro-benzoate
[(1R)-3-ethoxy-1-methyl-3-oxo-propyl] 5-bromo-2-chloro-4-fluoro-benzoate

### Step 3: Synthesis of [(1S)-3-ethoxy-1-methyl-3-oxo-propyl] 2-chloro-4-fluoro-5-(4,4,5,5-tetramethyl-1,3,2-dioxaborolan-2-yl)benzoate

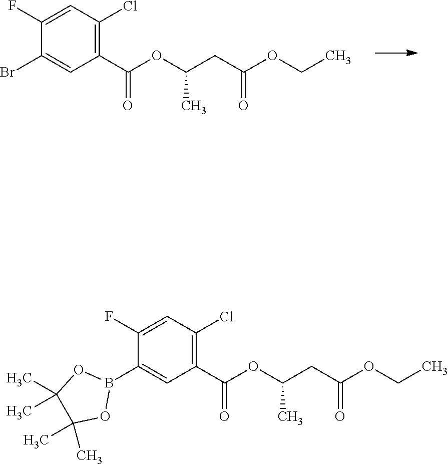

Potassium acetate (0.8 g, 8 mmol) and palladium(dppf dichloride (0.2 g, 0.27 mmol) were added to a stirred solution of [(1S)-3-ethoxy-1-methyl-3-oxo-propyl] 5-bromo-2-chloro-4-fluoro-benzoate (980 mg, 2.7 mmol) and bis(pinacolato)diboron (1.0 g, 4 mmol) in dioxane (20 ml). The resulting mixture was heated in a microwave oven at 100° C. for 45 minutes, then allowed to cool. Ethyl acetate (100 ml) was added, the mixture filtered and the filtrate evaporated under reduced pressure. The residue was purified by chromatography to provide [(1S)-3-ethoxy-1-methyl-3-oxo-propyl] 2-chloro-4-fluoro-5-(4,4,5,5-tetramethyl-1,3,2-dioxaborolan-2-yl)benzoate (650 mg).

Also prepared by this general method were:
[(1S)-3-ethoxy-1-methyl-3-oxo-propyl] 2-chloro-5-(4,4,5,5-tetramethyl-1,3,2-dioxaborolan-2-yl)benzoate
[(1R)-3-ethoxy-1-methyl-3-oxo-propyl] 2-chloro-4-fluoro-5-(4,4,5,5-tetramethyl-1,3,2-dioxaborolan-2-yl)benzoate

### Step 4: Synthesis of [(1S)-3-ethoxy-1-methyl-3-oxo-propyl] 2-chloro-5-[3-chloro-5-(trifluoromethyl)-2-pyridyl]-4-fluoro-benzoate (Compound 2-525; S-enantiomer)

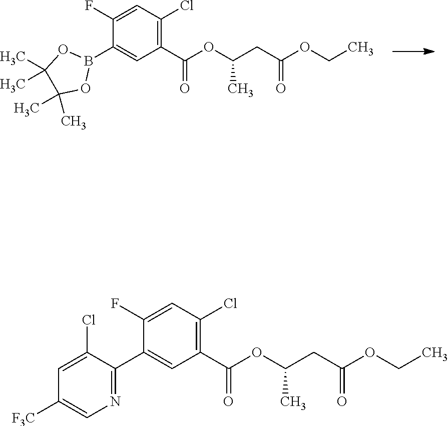 [SMILES: CCOC(=O)C[C@H](C)OC(=O)c1cc(B2OC(C)(C)C(C)(C)O2)c(F)cc1Cl ; CCOC(=O)C[C@H](C)OC(=O)c1cc(-c2ncc(C(F)(F)F)cc2Cl)c(F)cc1Cl]

Potassium acetate (0.23 g, 0.23 mmol) and palladium(dppf dichloride (55 mg, 0.075 mmol) were added to a stirred solution of [(1S)-3-ethoxy-1-methyl-3-oxo-propyl] 2-chloro-4-fluoro-5-(4,4,5,5-tetramethyl-1,3,2-dioxaborolan-2-yl)benzoate (620 mg, 0.75 mmol) and 2,3-dichloro-5-(trifluoromethyl)-pyridine (0.25 g, 1.1 mmol) in dioxane (12 ml). The resulting mixture was heated in a microwave oven at 100° C. for 45 minutes, then allowed to cool. Ethyl acetate (10 ml) was added, the mixture filtered and the filtrate evaporated under reduced pressure. The residue was purified by chromatography to provide [(1S)-3-ethoxy-1-methyl-3-oxo-propyl] 2-chloro-5-[3-chloro-5-(trifluoromethyl)-2-pyridyl]-4-fluoro-benzoate (Compound 2-525; S-enantiomer) as a gum (86 mg). ¹H NMR (400 MHz, CDCl₃) δ 8.9 (s, 1H), 8.1 (s, 1H), 8.0 (d, 1H), 7.35 (d, 1H), 5.55 (m, 1H), 4.15 (q, 2H), 2.8 (dd, 1H), 2.65 (dd, 1H), 1.45 (d, 3H), 1.2 (t, 3H) ppm.

Also prepared by this general method were:

[(1S)-3-ethoxy-1-methyl-3-oxo-propyl] 2-chloro-5-[3-chloro-5-(trifluoromethyl)-2-pyridyl]-benzoate (Compound 1-525; S-enantiomer): ¹H NMR (400 MHz, CDCl₃) δ 8.85 (s, 1H), 8.25 (s, 1H), 8.1 (s, 1H), 7.85 (d, 1H), 7.6 (d, 1H), 5.6 (m, 1H), 4.15 (q, 2H), 2.8 (dd, 1H), 2.65 (dd, 1H), 1.45 (d, 3H), 1.2 (t, 3H) ppm.

[(1R)-3-ethoxy-1-methyl-3-oxo-propyl] 2-chloro-5-[3-chloro-5-(trifluoromethyl)-2-pyridyl]-4-fluoro-benzoate (Compound 2-525; R-enantiomer): ¹H NMR (400 MHz, CDCl₃) δ 8.9 (s, 1H), 8.1 (s, 1H), 8.0 (d, 1H), 7.35 (d, 1H), 5.55 (m, 1H), 4.15 (q, 2H), 2.8 (dd, 1H), 2.6 (dd, 1H), 1.5 (d, 3H), 1.2 (t, 3H) ppm.

### Example 2 Preparation of (3-ethoxy-1-methyl-3-oxo-propyl) 2-chloro-5-[3-chloro-5-(trifluoromethyl)-2-pyridyl]benzoate (Compound 1-525)

### Step 1: Synthesis of methyl 2-chloro-5-[3-chloro-5-(trifluoromethyl)-2-pyridyl]benzoate

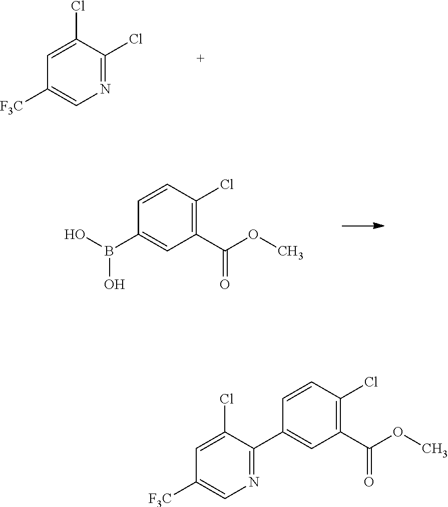 [SMILES: FC(F)(F)c1cnc(Cl)c(Cl)c1 ; COC(=O)c1cc(B(O)O)ccc1Cl ; COC(=O)c1cc(-c2ncc(C(F)(F)F)cc2Cl)ccc1Cl]

Potassium acetate (2.95 g, 30 mmol) and palladium(dppf) dichloride (730 mg, 0.98 mmol) were added to a stirred solution of (4-chloro-3-methoxycarbonyl-phenyl)-boronic acid (2.2 g, 9.8 mmol) and 2,3-dichloro-5-(trifluoromethyl)-pyridine (3.2 g, 15 mmol) in dioxane (13 ml). The resulting mixture was heated in a microwave oven at 100° C. for 45 minutes, then allowed to cool and evaporated. Dichloromethane (10 ml) was added, the mixture washed with water and evaporated under reduced pressure. The residue was purified by chromatography to provide methyl 2-chloro-5-[3-chloro-5-(trifluoromethyl)-2-pyridyl]benzoate as a pale orange oil (3.2 g). ¹H NMR (400 MHz, CDCl₃) δ 8.85 (s, 1H), 8.3 (d, 1H), 8.1 (s, 1H), 7.85 (dd, 1H), 7.6 (d, 1H), 3.95 (s, 3H) ppm.

### Step 2: Synthesis of 2-chloro-5-[3-chloro-5-(trifluoromethyl)-2-pyridyl]benzoic acid

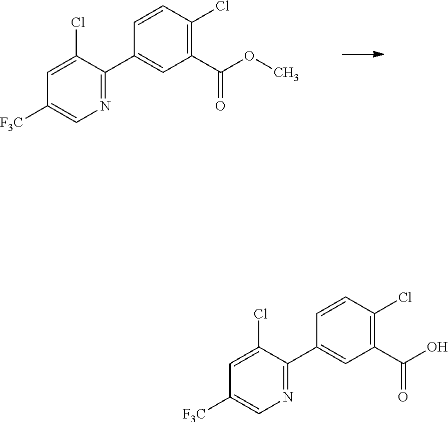 [SMILES: COC(=O)c1cc(-c2ncc(C(F)(F)F)cc2Cl)ccc1Cl ; O=C(O)c1cc(-c2ncc(C(F)(F)F)cc2Cl)ccc1Cl]

Aqueous sodium hydroxide (1N; 1.7 ml, 1.7 mmol) was added to a stirred solution of methyl 2-chloro-5-[3-chloro-5-(trifluoromethyl)-2-pyridyl]benzoate (300 mg, 0.86 mmol) in methanol (5 ml) at ambient temperature. The resulting mixture was stirred at ambient temperature for 17 hours, then evaporated under reduced pressure. Water was added to the residue and the mixture washed with ethyl acetate. Hydrochloric acid (2N) was added to bring the pH to 2, and the mixture extracted with ethyl acetate. The organic phase was dried over magnesium sulfate, filtered and evaporated under reduced pressure to provide 2-chloro-5-[3-chloro-5-(trifluoromethyl)-2-pyridyl]benzoic acid as a white solid (250 mg). ¹H NMR (400 MHz, CDCl₃) δ 8.9 (s, 1H), 8.35 (s, 1H), 8.15 (s, 1H), 7.9 (d, 1H), 7.6 (d, 1H) ppm (acid proton not observed).

### Step 3: Preparation of (3-ethoxy-1-methyl-3-oxo-propyl) 2-chloro-5-[3-chloro-5-(trifluoromethyl)-2-pyridyl]benzoate (Compound 1-525)

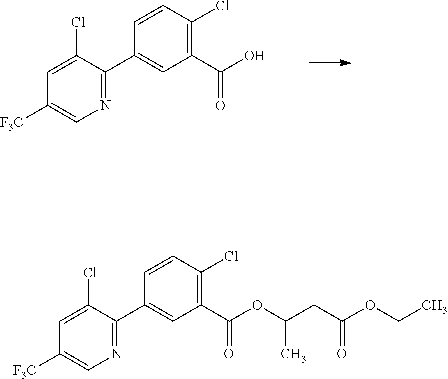 [SMILES: O=C(O)c1cc(-c2ncc(C(F)(F)F)cc2Cl)ccc1Cl ; CCOC(=O)CC(C)OC(=O)c1cc(-c2ncc(C(F)(F)F)cc2Cl)ccc1Cl]

Thionyl chloride (0.11 ml, 1.5 mmol) was added dropwise to a stirred solution of 2-chloro-5-[3-chloro-5-(trifluoromethyl)-2-pyridyl]benzoic (250 mg, 0.74 mmol) in toluene (5 ml) at ambient temperature. The resulting solution was heated at reflux for 1 hour, then allowed to cool. The solvent was evaporated under reduced pressure and the residue dissolved in dichloromethane (20 ml). This solution was added to a stirred solution of triethylamine (0.05 ml, 0.34 mmol) and ethyl 3-hydroxybutanoate (77 mg, 0.56 mmol) in dichloromethane (5 ml). The resulting mixture stirred at ambient temperature for 4 hours, then evaporated under reduced pressure and the residue purified by chromatography to provide (3-ethoxy-1-methyl-3-oxo-propyl) 2-chloro-5-[3-chloro-5-(trifluoromethyl)-2-pyridyl]benzoate (Compound 1-525) as an oil (30 mg). ¹H NMR (400 MHz, CDCl₃) δ 8.85 (s, 1H), 8.25 (d, 1H), 8.1 (s, 1H), 7.85 (dd, 1H), 7.6 (d, 1H), 5.6 (m, 1H), 4.15 (q, 2H), 2.8 (dd, 1H), 2.65 (dd, 1H), 1.45 (d, 3H), 1.2 (t, 3H) ppm.

### Example 3 Preparation of [(1S)-3-methoxy-1-methyl-3-oxo-propyl] 3-chloro-6-[3-chloro-5-(trifluoromethyl)-2-pyridyl]-5-fluoro-pyridine-2-carboxylate (Compound 11-524; S-enantiomer)

### Step 1: Synthesis of [(1S)-3-methoxy-1-methyl-3-oxo-propyl] 6-bromo-3-chloro-5-fluoro-pyridine-2-carboxylate

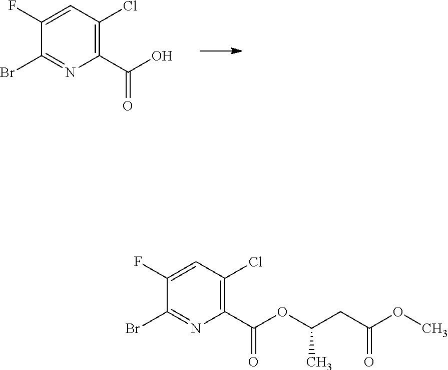 [SMILES: O=C(O)c1nc(Br)c(F)cc1Cl ; COC(=O)C[C@H](C)OC(=O)c1nc(Br)c(F)cc1Cl]

Oxalyl chloride (1 ml, 12 mmol) was added dropwise to a stirred solution of 6-bromo-3-chloro-5-fluoro-pyridine-2-carboxylic acid (1.5 g, 5.9 mmol) in dichloromethane (30 ml) at ambient temperature. The resulting solution was stirred at 0° C. for 5 mins, then dimethylformamide (3 drops) was added and the resulting solution stirred at ambient temperature for a further 2 hours. The solvent was evaporated under reduced pressure and the residue dissolved in acetonitrile (12 ml). This solution was added dropwise to a solution of pyridine (2.4 ml, 30 mmol) and ethyl (3S)-3-hydroxybutanoate (0.98 g, 8.3 mmol) in acetonitrile (30 ml). The resulting mixture stirred at 0° C. for 10 minutes, then at ambient temperature for 17 hours. Water was added, followed by concentrated hydrochloric acid to bring the pH to 1. The mixture was extracted twice with ethyl acetate and the combined organic extracts washed with brine, dried over magnesium sulphate, filtered and evaporated under reduced pressure to provide [(1S)-3-methoxy-1-methyl-3-oxo-propyl] 6-bromo-3-chloro-5-fluoro-pyridine-2-carboxylate (2.1 g). ¹H NMR (400 MHz, CDCl₃) δ 7.55 (d, 1H), 5.6 (m, 1H), 3.7 (s, 3H), 2.85 (dd, 1H), 2.65 (dd, 1H), 1.5 (d, 3H) ppm.

### Step 2: Synthesis of [(1S)-3-methoxy-1-methyl-3-oxo-propyl] 3-chloro-6-[3-chloro-5-(trifluoromethyl)-2-pyridyl]-5-fluoro-pyridine-2-carboxylate (Compound 11-524; S-enantiomer)

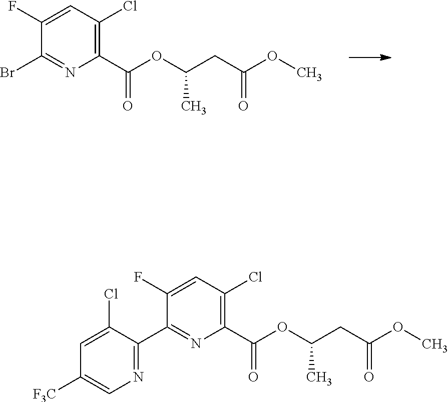 [SMILES: COC(=O)C[C@H](C)OC(=O)c1nc(Br)c(F)cc1Cl ; COC(=O)C[C@H](C)OC(=O)c1nc(-c2ncc(C(F)(F)F)cc2Cl)c(F)cc1Cl]

[(1S)-3-Methoxy-1-methyl-3-oxo-propyl] 6-bromo-3-chloro-5-fluoro-pyridine-2-carboxylate (0.25 g, 0.71 mmol), bis(triphenylphosphine)palladium dichloride (40 mg, 0.06 mmol) and copper(I) iodide (21 mg, 0.11 mmol) were added to a stirred solution of [3-chloro-5-(trifluoromethyl)-2-pyridyl]-trimethyl-stannane (prepared as described in Example 4; 640 mg, 1.1 mmol) in toluene (5 ml). The resulting mixture was heated at 100° C. for 2.5 hours, then allowed to cool and evaporated. The residue was purified by chromatography to provide [(1S)-3-methoxy-1-methyl-3-oxo-propyl] 3-chloro-6-[3-chloro-5-(trifluoromethyl)-2-pyridyl]-5-fluoro-pyridine-2-carboxylate (Compound 11-524; S-enantiomer) as a yellow oil (184 mg). ¹H NMR (400 MHz, CDCl₃) δ 8.9 (s, 1H), 8.1 (s, 1H), 7.75 (d, 1H), 5.6 (m, 1H), 3.7 (s, 3H), 2.85 (dd, 1H), 2.65 (dd, 1H), 1.5 (d, 3H) ppm.

### Example 4 Preparation of [3-chloro-5-(trifluoromethyl)-2-pyridyl]-trimethyl-stannane

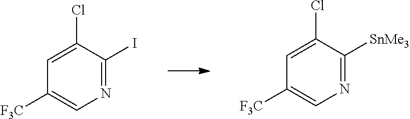

3-Chloro-2-iodo-5-(trifluoromethyl)-pyridine (1.0 g, 3.3 mmol) and bis(triphenylphosphine)palladium dichloride (230 mg, 0.33 mmol) were added to a stirred solution of hexamethylditin (1.3 g, 3.9 mmol) in dioxane (10 ml). The resulting mixture was heated at 100° C. for 2 hours, then allowed to cool, filtered and evaporated under reduced pressure to provide [3-chloro-5-(trifluoromethyl)-2-pyridyl]-trimethyl-stannane as a brown oil (2.0 g). ¹H NMR (400 MHz, CDCl₃) δ 8.85 (s, 1H), 7.75 (s, 1H), 0.45 (t, 9H) ppm.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xvlene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol | 6% |
| ether (15 mol of ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

### Biological Examples

### Pre-Emergence Biological Efficacy

Seeds of weeds and/or crops were sown in standard soil in pots (Amaranthus palmeri (AMAPA), Lolium perenne (LOLPE), Euphorbia heterophylla (EPHHL), Setaria faberi (SETFA), Echinochloa crus-galli (ECHCG), Ipomoea hederacea (IPOHE)). After cultivation for one day under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted using 0.2% Genapol X080 as diluent to give the desired final dose of test compound. Compounds were applied at a 250 g/ha unless otherwise stated. The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 2 below.

TABLE 2

| | Species | | | | | |
|---|---|---|---|---|---|---|
| Compound | AMAPA | LOLPE | EPHHL | SETFA | ECHCG | IPOHE |
| 1-525 | 100 | 100 | 100 | 100 | 100 | 70 |
| 1-525 (S- | 100 | 100 | 100 | 100 | 100 | 70 |
| enantiomer) | | | | | | |
| 2-525 (R- | 100 | 100 | 100 | 100 | 100 | 60 |
| enantiomer) | | | | | | |
| 2-525 (S- | 100 | 100 | 100 | 100 | 100 | 100 |
| enantiomer) | | | | | | |
| 11-524 (S- | 100 | 100 | 100 | 100 | 100 | 100 |
| enantiomer) | | | | | | |

### Post-Emergence Biological Efficacy

Seeds of weeds and/or crops were sown in standard soil in pots (Amaranthus palmeri (AMAPA), (CHEAL), Euphorbia heterophylla (EPHHL), Ipomoea hederacea (IPOHE), Eleusine indica (ELEIN), Lolium perenne (LOLPE), Digitaria sanguinalis (DIGSA), Setaria faberi (SETFA), Echinochloa crus-galli (ECHCG)). After cultivation for 14 days under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted using 0.2% Genapol XO80 as diluent to give the desired final dose of test compound. Compounds were applied at a 250 g/ha unless otherwise stated. The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 3 below.

TABLE 3

| | Species | | | | | | | | |
|---|---|---|---|---|---|---|---|---|---|
| Compound | AMAPA | CHEAL | EPHHL | IPOHE | ELEIN | LOLPE | DIGSA | SETFA | ECHCG |
| 1-525 | 100 | 100 | 100 | 80 | 100 | 100 | 100 | 100 | 100 |
| 1-525 (S- | 100 | 100 | 100 | 90 | 100 | 100 | 100 | 100 | 100 |
| enantiomer) | | | | | | | | | |
| 2-525 (R- | 100 | 100 | 100 | 100 | 100 | 100 | 100 | 100 | 100 |
| enantiomer) | | | | | | | | | |
| 2-525 (S- | 100 | 100 | 100 | 100 | 100 | 100 | 100 | 100 | 100 |
| enantiomer) | | | | | | | | | |
| 11-524 (S- | 100 | 100 | 100 | 100 | 100 | 100 | 100 | 100 | 100 |
| enantiomer) | | | | | | | | | |

1. A compound of formula (I) or an agronomically acceptable salt thereof: 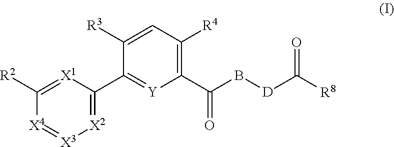
wherein
X¹ is selected from C—R¹, nitrogen and N⁺—O⁻;
X² is selected from C—R¹⁷ and nitrogen;
X³ is selected from C—R¹⁸ and nitrogen;
X⁴ is selected from C—R¹⁹ and nitrogen;
with the proviso that a maximum of two of X¹, X², X³ and X⁴ are nitrogen, and X³ and X⁴ are not both nitrogen;
Y is selected from C—H and nitrogen;
B is selected from O, S and NR⁵;
D is (CR⁶R⁷)ₙ;
n is an integer from 1 to 4;
R¹ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹ and CONR¹⁰R¹¹;
R² is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹, and CONR¹⁰R¹¹; or
R¹ and R² together with the carbon atoms to which they are attached form a 5- or 6-membered ring, which may be saturated or partially or fully unsaturated, and which may optionally contain one or two heteroatoms selected from nitrogen, oxygen and sulfur, and which may be substituted with 1 to 4 groups represented by R¹⁶; or
R² and R¹⁹ together with the carbon atoms to which they are attached form a 5- or 6-membered ring, which may be saturated or partially or fully unsaturated, and which may optionally contain one or two heteroatoms selected from nitrogen, oxygen and sulfur, and which may be substituted with 1 to 4 groups represented by R¹⁶;
R³ is selected from hydrogen, halogen, C₁-C₄alkyl, C₁-C₄haloalkyl, C₁-C₄alkoxy, C₁-C₄haloalkoxy, C₁-C₄alkylthio and C₁-C₄alkylsulfonyl;
R⁴ is selected from hydrogen, halogen, cyano, nitro, aminocarbonyl, aminothiocarbonyl, C₁-C₄alkyl, C₁-C₄haloalkyl, C₁-C₄alkoxy, C₁-C₄haloalkoxy and C₁-C₄alkylsulfonyl;
R⁵ is selected from the group consisting of hydrogen, hydroxy, C₁-C₆alkyl and C₁-C₄alkoxy;
each R⁶ and R⁷ is independently selected from hydrogen, halogen, C₁-C₆alkyl, C₁-C₆haloalkyl, hydroxy, C₁-C₄alkoxy, C₁-C₄alkoxycarbonyl and CH₂OR¹²; provided that R⁶ and R⁷ are not both hydroxy on the same carbon atom;
or two groups R⁶ and R⁷, on the same or different carbon atoms, together form a C₁-C₅alkylene chain, which contain 0, 1 or 2 oxygen atoms, substituted by 1 to 3 groups represented by R¹⁵;
or two groups R⁶ and R⁷, together with the carbon atom to which they are attached may form a C₂alkene;
R⁸ is selected from OR⁹, SR⁹ and NR¹⁰R¹¹;
R⁹ is selected from hydrogen, C₁-C₁₀alkyl, C₁-C₁₀haloalkyl, C₃-C₆alkenyl, C₃-C₆haloalkenyl, C₃-C₆alkynyl, C₁-C₄alkoxyC₁-C₆alkyl, C₁-C₄haloalkoxyC₁-C₆alkyl, C₆-C₁₀arylC₁-C₃alkyl, C₆-C₁₀arylC₁-C₃alkyl substituted by 1 to 4 groups represented by R¹³, heteroarylC₁-C₃alkyl and heteroarylC₁-C₃alkyl substituted by 1 to 3 groups represented by R¹³;
R¹⁰ is selected from hydrogen, C₁-C₆alkyl and SO₂R¹⁴;
R¹¹ is selected from hydrogen and C₁-C₆alkyl; or
R¹⁰ and R¹¹ together with the nitrogen to which they are attached form a 3- to 6-membered heterocyclyl ring, which optionally contains an oxygen atom;
R¹² is selected from hydrogen, C₁-C₄alkyl and C₁-C₄alkylcarbonyl;
each R¹³ is independently selected from halogen, C₁-C₄alkyl, C₁-C₄haloalkyl, C₁-C₄alkoxy, C₁-C₄haloalkoxy, cyano and C₁-C₄alkylsulfonyl;
R¹⁴ is selected from the group consisting of C₁-C₄alkyl, C₁-C₄haloalkyl, and C₁-C₄alkyl(C₁-C₄alkyl)amino;
each R¹⁵ is independently selected from hydrogen, halogen, C₁-C₄alkyl and C₁-C₄haloalkyl;
R¹⁶ is selected from halogen, C₁-C₄alkyl, C₁-C₄haloalkyl, C₁-C₄alkoxy, C₁-C₄haloalkoxy, cyano and C₁-C₄alkylsulfonyl;
R¹⁷ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹ and CONR¹⁰R¹¹;
R¹⁸ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹ and CONR¹⁰R¹¹;
R¹⁹ is selected from hydrogen, halogen, cyano, nitro, C₁-C₄alkyl, C₁-C₄haloalkyl, C₃-C₆cycloalkyl, C₁-C₄alkoxyC₁-C₆alkyl, C₁-C₄haloalkoxyC₁-C₆alkyl, C₁-C₄alkoxy, C₁-C₄haloalkoxy, C₁-C₄alkoxyC₁-C₄alkoxy, C₁-C₄alkylsulfonyloxy, C₁-C₄haloalkylsulfonyloxy, C₁-C₄alkylthio, C₁-C₄alkylsulfinyl, C₁-C₄alkylsulfonyl, C₁-C₄haloalkylthio, C₁-C₄haloalkylsulfinyl, C₁-C₄haloalkylsulfonyl, amino, C₁-C₄alkylamino, di(C₁-C₄alkyl)amino, C₁-C₄alkylcarbonylamino, C₁-C₄alkylcarbonyl(C₁-C₄alkyl)amino, C₁-C₄alkyloxycarbonylamino, aminocarbonylamino, C₁-C₄alkylaminocarbonylamino, C₁-C₄alkylsulfonylamino, C₁-C₄haloalkylsulfonylamino, CO₂R⁹ and CONR¹⁰R¹¹; and
with the proviso that R¹, R², R¹⁷, R¹⁸ and R¹⁹ are not all hydrogen.

2. A compound as claimed in claim 1 in which X¹ is C—R¹.

3. A compound as claimed in claim 1 in which X² is nitrogen.

4. A compound as claimed in claim 1 in which X³ is C—R¹⁸.

5. A compound as claimed in claim 1 in which X⁴ is C—R¹⁹.

6. A compound as claimed in claim 1 in which Y is C—H.

7. A compound as claimed in claim 1 in which B is O.

8. A compound as claimed in claim 1 in which n is 2.

9. A compound as claimed in claim 1 in which R² is selected from hydrogen, fluorine, chlorine, and trifluoromethyl.

10. A compound as claimed in claim 1 in which R³ is selected from hydrogen, chlorine and fluorine.

11. A compound as claimed in claim 1 in which R⁴ is selected from hydrogen, chlorine, bromine, cyano and aminothiocarbonyl, more preferably chlorine, bromine and cyano.

12. A compound as claimed in claim 1 in which R⁸ is OR⁹.

13. An agrochemical composition comprising a herbicidally effective amount of a compound of formula (I) as defined in claim 1 and an agrochemically-acceptable diluent or carrier.

14. A method of controlling or preventing undesirable plant growth, wherein a herbicidally effective amount of a compound of formula (I) as defined in claim 1 is applied to the plants, to parts thereof or to the locus thereof.

15. A method for controlling undesired vegetation at a plant cultivation site, the method comprising the steps of: a) providing, at said site, a plant that comprises at least one nucleic acid comprising a nucleotide sequence encoding a protoporphyrinogen oxidase (PPO) polypeptide which is resistant or tolerant to a “PPO inhibiting herbicide”; b) applying to said site an effective amount of said herbicide, wherein the PPO inhibiting herbicide is a compound as claimed in claim 1.
